(19) United States
(12) Patent Application Publication
(10) Pub. No.: US 2025/0228908 A1
(43) Pub. Date: Jul. 17, 2025

(54) TREATING ORTHOPEDIC INJURY WITH A VECTOR DRIVING EXPRESSION OF ACID CERAMIDASE

(71) Applicant: Icahn School of Medicine at Mount Sinai, New York, NY (US)

(72) Inventors: Efrat ELIYAHU, New York, NY (US); Michael G. KATZ, New York, NY (US); Adam VINCEK, New York, NY (US)

(73) Assignee: Icahn School of Medicine at Mount Sinai, New York, NY (US)

(21) Appl. No.: 18/852,642

(22) PCT Filed: Mar. 31, 2023
(86) PCT No.: PCT/US2023/065223

§ 371 Date: Sep. 30, 2024

Related U.S. Application Data
(60) Provisional application No. 63/326,481, filed on Apr. 1, 2022.

(51) Int. Cl. A61K 35/76; A61K 38/50; A61K 48/00; A61P 19/02; A61P 19/04; C12N 9/80; C12N 15/86

(52) U.S. Cl. CPC A61K 35/76; A61K 38/50; A61K 48/005; A61K 48/0075; A61P 19/02; A61P 19/04; C12N 9/80; C12N 15/86; C12Y 305/01023

(57) ABSTRACT

The present disclosure relates to a method of treating an orthopedic condition, an orthopedic injury, or both in a subject in need of such treatment. The method includes administering a therapeutically effective amount of an adeno-associated viral vector to the subject. The adeno-associated viral vector transfects cells with a polynucleotide sequence that encodes and drives expression of an acid ceramidase. Also disclosed is a method of treating orthopedic inflammation in a subject in need of such treatment. The method includes administering a therapeutically effective amount of an adeno-associated viral vector that codes for expression of acid ceramidase to the subject.

### CROSS-REFERENCE TO RELATED APPLICATIONS

This application claims priority to U.S. provisional application No. 63/326,481, the entire content of which is incorporated herein by reference.

### SEQUENCE LISTING

The instant application contains an electronic sequence listing. The contents of the electronic sequence listing H2623637.xml; Size: 112,987 bytes; and date of creation: Mar. 16, 2023; is herein incorporated by reference in its entirety.

### FIELD

The present disclosure relates to methods for treating an orthopedic condition or injury and reducing and preventing inflammation associated with orthopedic injuries or conditions.

### BACKGROUND

Orthopedic disorders are one of the leading causes of morbidity in mammals. Their prevalence increases dramatically with age and weight gain. Many orthopedic conditions are good candidates for gene therapy. In humans, gene therapy clinical trials have already been initiated for chronic orthopedic diseases.

Osteoarthritis (AO) or degenerative joint disease (DJD) injuries or development/genetic problems leading to AO is a societal problem. Similarly, injuries to bones, joints, tendons, ligaments, and the spinal cord are often difficult to prevent or treat.

These diseases and injuries result in damage to joint cartilage and tissue, and fluid surrounding joint cartilage deteriorate. Over time, this leads to bone-on-bone contact, chronic inflammation, swelling, pain, and loss of mobility. Mild cases include decreased interest in activities that require mobility, including walking and recreational activities. Moderate cases include limping, struggling to get up, lying down, or refusing to climb stairs. Severe cases include a loss in the ability to walk or function.

Joint supplements and pain control is a mainstay of osteoarthritis treatment. The most commonly used pain control medications for more severe osteoarthritis are Non-Steroidal Anti-Inflammatory Drugs (NSAIDs). NSAIDs impose risks and often result in undesirable side effects. In addition, stem cell therapy in the past six years has shown limited improvement and includes major surgery and repeated follow up injections.

The present disclosure is directed to overcoming these and other deficiencies in the art.

### BRIEF DESCRIPTION

In an aspect, disclosed is a method of treating an orthopedic condition, an orthopedic injury, or both in a subject in need of such treatment, including administering a therapeutically effective amount of an adeno-associated viral vector to the subject, wherein the adeno-associated viral vector transfects cells with a polynucleotide sequence that encodes and drives expression of an acid ceramidase.

In an example, one or both of the orthopedic condition and the orthopedic injury include inflammation. In another example, one or both of the orthopedic condition and the orthopedic injury includes joint inflammation. In still another example, one or both of the orthopedic condition and the orthopedic injury includes spinal inflammation. In yet another example, one or both of the orthopedic condition and the orthopedic injury includes a joint condition or a joint injury.

In a further example, one or both of the orthopedic condition and the orthopedic injury includes arthritis, bursitis, a degenerative joint and bone disease, fibromyalgia, a fracture, back pain, hand pain, knee pain, neck pain, hip pain, shoulder pain, kyphosis, a joint dislocation, a joint tear, a joint fracture, a joint break, gout, osteoporosis, Paget's disease, rheumatic disorder, a joint sprain, a joint strain, scoliosis, tendonitis, soft tissue injury, dysplasia, ligament injury, or any combination of two or more of the foregoing.

In still a further example, one or both of the orthopedic condition and the orthopedic injury includes a degenerative joint and bone disease, said disease is selected from osteoarthritis, rheumatoid arthritis, psoriatic arthritis, ankylosing spondylitis, or any combination of two or more of the foregoing.

In yet a further example, the administering includes a single administration of said adeno-associated viral vector. In another example, the administering includes intravenous injection, intramuscular injection, subcutaneous injection, intradermal injection, intraosseous injection, localized injection, long-acting injection formulation, depot injection, implantable injection, endotracheal injection, intra-arterial injection, intrathecal, or any combination of two or more of the foregoing. In yet another example, the adeno-associated viral vector is administered in an amount between about 1¹⁰ and about 1¹⁴ particles per administration.

In a further example, the adeno-associated viral vector is selected from Anc80, Anc80L65, AAV6, AAV1, AAV2, AAV8, AAV9, or any combination of two or more of the foregoing.

In still a further example, the acid ceramidase includes an amino acid sequence as set out in SEQ ID NO: 27, SEQ ID. NO: 28, or SEQ ID: 29. In yet a further example, the acid ceramidase includes an amino acid sequence as set out in SEQ ID NO: 27.

In another aspect, disclosed is a method of treating orthopedic inflammation in a subject in need of such treatment including administering a therapeutically effective amount of an adeno-associated viral vector to the subject, where the adeno-associated viral vector transfects cells with a polynucleotide sequence that encodes and drives expression of an acid ceramidase.

In an example, the inflammation includes joint inflammation. In another example, the inflammation includes spinal inflammation. In still another example the orthopedic inflammation is caused by one or both of a joint condition and a joint injury. In yet another example, the orthopedic inflammation includes or is caused by arthritis, bursitis, a degenerative joint and bone disease, fibromyalgia, a fracture, back pain, hand pain, knee pain, neck pain, hip pain, shoulder pain, kyphosis, a joint dislocation, a joint tear, a joint fracture, a joint break, gout, osteoporosis, Paget's disease, rheumatic disorder, a joint sprain, a joint strain, scoliosis, tendonitis, soft tissue injury, dysplasia, ligament injury, or any combination of two or more of the foregoing. In a further example, the orthopedic inflammation is selected from osteoarthritis, rheumatoid arthritis, psoriatic arthritis, ankylosing spondylitis, or any combination of two or more of the foregoing.

In still a further example, administering includes a single administration of the adeno-associated viral vector. In yet a further example, administering includes intravenous injection, intramuscular injection, subcutaneous injection, intradermal injection, intraosseous injection, localized injection, long-acting injection formulation, depot injection, implantable injection, endotracheal injection, intra-arterial injection, intrathecal, or any combination of two or more of the foregoing. In another example, the adeno-associated viral vector is administered in an amount between about 1¹⁰ and about 1¹⁴ particles per administration.

In still another example, the adeno-associated viral vector is selected from Anc80, Anc80L65, AAV6, AAV1, AAV2, AAV8, AAV9, or any combination thereof. In yet another example, the acid ceramidase includes an amino acid sequence as set out in SEQ ID NO: 27, SEQ ID NO: 28, or SEQ ID NO: 29. In a further example, the acid ceramidase includes an amino acid sequence as set out in SEQ ID NO: 27.

In still a further example, the administering results in improved ambulation or mobility. In yet a further example, the administering results in pain reduction. In yet a further example, the administering results in faster wound healing than without the administering.

### BRIEF DESCRIPTION OF THE DRAWINGS

FIG. 1 shows impact on pain score on test subjects (before treatment and after treatment).

FIG. 2 shows pain dynamics summary data on test subjects (before treatment and after treatment).

FIG. 3 illustrates a joint injury that may be treated in accordance with the methods of the present disclosure.

FIG. 4 illustrates an osteoarthritis ambulation deficit score on 14 test subjects (on the day of treatment and after treatment).

### DETAILED DESCRIPTION

An aspect of the present disclosure relates to a method of treating an orthopedic condition, an orthopedic injury, or both in a subject in need of such treatment, including administering a therapeutically effective amount of an adeno-associated viral vector to the subject, wherein the adeno-associated viral vector transfects cells with a polynucleotide sequence that encodes and drives expression of an acid ceramidase.

An aspect of the present disclosure relates to a method of treating orthopedic inflammation in a subject in need of such treatment, including administering a therapeutically effective amount of an adeno-associated viral vector that codes for expression of acid ceramidase to the subject.

The present disclosure provides a method for treating and/or repairing tissue post-acute or chronic orthopedic injuries, orthopedic conditions and post-surgery by administering to a subject a treatment to increase acid ceramidase (AC). For example, a vector may be administered to a subject wherein the vector transfects cells with a polynucleotide encoding a ceramidase and drives expression of the ceramidase in transfected cells. The vector may be a viral vector, such as an adeno-associated viral vector (AAV). Examples may include an Anc80 AAV vector, including Anc80 variants such as, in a non-limiting example, Anc80L65.

Acid ceramidase (AC) is a sole enzyme that can regulate ceramide hydrolysis to prevent cell death and leads to synthesize Sphingosine 1 Phosphate (S1P) from Sphingosine to initiate cell survival. AC gene therapy may prevent senescence and the progression of tissue damage and scarring. AC expression also initiates expression of factors that lead to tissue repair. Included in this disclosure are results of pre-clinical trials testing treatment of orthopedic injuries or conditions or inflammation associated therewith. The present disclosure demonstrates the use of AC gene therapy for tissue repair, treating orthopedic conditions, orthopedic injury and surgeries, and associated inflammation. Anc80 virus, an in silico designed gene therapy vector, has demonstrated high gene expression levels in the liver, eye and ear compared to naturally-occurring adeno-associated viral vectors (AAVs) that are currently in clinical development. Anc80, an engineered gene therapy vector, is synthetic in nature it is not known to circulate in humans, making it less likely to be recognized immunologically by antibodies against naturally occurring AAVs.

A technology platform has been developed using Anc80 Viral vector encoding acid ceramidase. This platform demonstrated positive results in multiple clinical disorders involved in inflammatory degenerative conditions including cardiovascular, orthopedic, and neurology diseases. Addition of recombinant AC to primary chondrocyte culture media has been shown to maintain low levels of ceramide. After three weeks of expansion the chondrogenic phenotype of these cells also was markedly improved, as assessed by a combination of histochemical staining, and molecular analysis. The same effects were evident in rat, equine and human cells, and were observed in monolayer and 3-D cultures. AC also reduced the number of apoptotic cells in some culture conditions, contributing to overall improved cell quality. In addition to these effects on primary chondrocytes, when AC was added to freshly harvested rat, equine or feline bone marrow cultures a 2-fold enrichment of mesenchymal stem cells (MSCs) was observed by one week. AC also improved the chondrogenic differentiation of MSCs. Supplementation of chondrocyte media with recombinant acid ceramidase (rhAC) also influences cartilage repair in a rat osteochondral defect model using stem cell therapy.

This technology includes in vitro use of AC protein with growing primary chondrocytes in culture with rhAC for 10 days. The cells were then introduced into osteochondral defects created in Sprague Dawley rat trochlea by a micro-drilling procedure. Treatment with rhAC led to increased cell numbers and glycosaminoglycan (GAG) production following 7 days of expansion in vitro. Gene expression of collagen 2, aggrecan and Sox-9 also was significantly elevated. At 6 weeks post-surgery, defects containing rhAC-treated cells exhibited more soft tissue formation at the articular surface, as evidenced by microCT, as well as histological evidence of enhanced cartilage repair. The results support the positive effects of rhAC treatment on chondrocyte growth and phenotype in vitro, and reveal for the first time the in vivo effects of the treated cells on cartilage repair. AC gene therapy may be used to improve the outcome of ligament rapture, bone fracture and cartilage damage in chronic disease. An embodiment of the technology presented in this disclosure relates to injecting a vector driving expression of AC into injured areas following post-acute orthopedic injury such as ligament rapture and to initiate rapid repair.

An example in accordance with the present disclosure includes injection to an injured area and may not require surgeries or use of cell culture. This is a useful embodiment of an in vivo application to induce endogenous tissue repair alone or in combination with surgeries, medications or cell therapy. It is clinically relevant to execute gene delivery in large animals and human and a vector driving expression of acid ceramidase addresses the problem of scale, to achieve very high gene transduction levels using surgical methods. A pre-clinical study was conducted in dogs using, in an example, Anc80.AC virus to repair tear ligament and cartilage repair in chronic disease. The results showed extremely fast recovery from ligament tear injury (two weeks). Inflammation, reduced by 50% in as fast as 48 hours post treatment, including less pain and more ambulation. Blood work post 7 days confirmed minimal inflammation and no toxicity. The progress of healing as reported by the orthopedic veterinarian was more than two times as fast.

An embodiment in accordance with the present disclosure includes gene therapy via cartilage injection. Administration with adeno-associated virus (AAV) is a novel, robust treatment option for osteoarthritis (OA) since genes can effectively address root cause disease mechanisms in the inflammatory condition. Surviving chondrocytes and synoviocytes create new cartilage, fibrocytes repair the ligaments and osteocytes repair the bones density. One embodiment of gene therapy works by safely transferring an episomal (i.e. not integrated) DNA instruction for lifelong expression in a one-time treatment.

As disclosed herein, compositions and methods of the present disclosure reduce inflammation and decrease pain, and rescue cells from senescence and lead to restore normal cell function; reverse senescence, inhibits the release of cytokines reducing inflammation and help to restore normal function. The compositions and methods of the present disclosure also restore cell function leading to activity of normal existing cartilage components and improves synovial fluid to help renew joint mobility.

A first aspect relates to a method of repairing damaged fluid, organ, tissue, and/or cell. The method includes administering a therapeutically effective amount of an adeno-associated viral vector that codes for expression of acid ceramidase to a damaged fluid, organ, tissue, and/or cell of a subject under conditions effective to repair damaged fluid, organ, tissue, and/or cell.

It is to be appreciated that certain aspects, modes, embodiments, variations, and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this disclosure belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±1 or ±10%, or any point therein, and remain within the scope of the disclosed embodiments.

Where a range of values is described, it should be understood that intervening values, unless the context clearly dictates otherwise, between the upper and lower limit of that range and any other stated or intervening value in other stated ranges, may be used in the embodiments described herein.

As used herein, the terms “subject”, “individual”, or “patient,” are used interchangeably, and mean any animal, including mammals, such as mice, rats, other rodents, rabbits, dogs, cats, swine, cattle, sheep, horses, or primates, such as humans.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

The term “cell or group of cells” is intended to encompass single cells as well as multiple cells either in suspension or in monolayers. Whole tissues also constitute a group of cells. Suitable cells populations according to this aspect of the present disclosure include mammalian cells populations, e.g., human cells populations, equine cells populations, porcine cells populations, feline cells populations, and/or canine cells populations. Human cells populations are particularly preferred.

The term “inhibit” or “inhibition” when used in conjunction with a discussion of senescence includes the ability of the sphingolipid-metabolizing proteins of the disclosure to reverse senescence, thereby returning to normal or near normal function.

The terms “stress”, “stress-related events” or “cellular-stress” refers to a wide range of molecular changes that cells undergo in response to environmental stressors, such as extreme temperatures, exposure to toxins, mechanical damage, anoxia, and noise.

The term “treating” or “to treat” means reducing or preventing, such as reducing or preventing inflammation, pain, lameness, and conditions associated with injuries and conditions. Treating also means administering as described in the present disclosure. Treating also means to provide medical care.

The term “biolistics” is short for “biological ballistics” and also known as particle-mediated gene transfer, is the method of directly shooting DNA or RNA into cells.

Ceramidases hydrolyze the amide linkage of ceramides to generate free fatty acids and sphingoid bases (Nikolova-Karakashian et al., Methods Enzymol. 311:194-201 (2000), Hassler et al., Adv. Lipid Res. 26:49-57 (1993), which are hereby incorporated by reference in their entirety). There are three types of ceramidases described (Nikolova-Karakashian et al., Methods Enzymol. 311:194-201 (2000), which is hereby incorporated by reference in its entirety). These are classified as acid, neutral, and alkaline ceramidases according to their pH optimum of enzymatic activity. Vectors described herein may code for expression of ceramidases that are suitable for use in this and all aspects of the present disclosure including acid ceramidase (AC), neutral ceramidase, alkali ceramidase, and other ceramidases. In all aspects of the present disclosure, the ceramidase can be homologous (i.e., derived from the same species) or heterologous (i.e., derived from a different species) to the tissue, cells, and/or subject being treated.

Acid ceramidases have optimal enzymatic activity at a pH of 1-5. The murine acid ceramidase was the first ceramidase to be cloned (Koch et al., J. Biol. Chem. 271:33110-33115 (1996), which is hereby incorporated by reference in its entirety). It is localized in the lysosome and is mainly responsible for the catabolism of ceramide. Dysfunction of this enzyme because of a genetic defect leads to a sphingolipidosis disease called Farber disease (Koch et al., J. Biol. Chem. 271:33110-33115 (1996), which is hereby incorporated by reference in its entirety).

Neutral ceramidases have been purified from rat brain (El Bawab et al., J. Biol. Chem 274:27948-27955 (1999), which is hereby incorporated by reference in its entirety) and mouse liver (Tani et al., J. Biol. Chem 275:3462-3468 (2000), which is hereby incorporated by reference in its entirety), and were cloned from Pseudomonas (Okino et al., J. Biol. Chem. 274:36616-36622 (1999), which is hereby incorporated by reference in its entirety), mycobacterium (Okino et al., J. Biol. Chem. 274:36616-36622 (1999), which is hereby incorporated by reference in its entirety), mouse (Tani et al., J. Biol. Chem 275:11229-11234 (2000), which is hereby incorporated by reference in its entirety), and human (El Bawab et al., J. Biol. Chem. 275:21508-21513 (2000), which is hereby incorporated by reference in its entirety). These ceramidases share significant homology, and this homology extends to putative proteins deduced from expressed sequence tag (EST) sequences of Dictyostelium discoideum and Arabidopsis thaliana (Okino et al., J. Biol. Chem. 274:36616-36622 (1999), El Bawab et al., J. Biol. Chem. 275:21508-21513 (2000), which are hereby incorporated by reference in their entirety). These ceramidases have a broad pH optimum ranging from 5 to 9 for their activity (Tani et al., J. Biol. Chem 275:11229-11234 (2000), El Bawab et al., J. Biol. Chem. 275:21508-21513 (2000), which are hereby incorporated by reference in their entirety). They appear to hydrolyze unsaturated ceramide preferentially, saturated ceramide (dihydroceramide) slightly, and hardly hydrolyze phytoceramide (Tani et al., J. Biol. Chem 275:11229-11234 (2000), which is hereby incorporated by reference in its entirety). The Pseudomonas, mouse, and human neutral ceramidases have a reverse ceramidase activity of catalyzing the formation of ceramide from sphingosine and a fatty acid (Okino et al., J. Biol. Chem. 274:36616-36622 (1999), Tani et al., J. Biol. Chem 275:11229-11234 (2000), Kita et al., Biochim. Biophys. Acta 1485:111-120 (2000), which are hereby incorporated by reference in their entirety). El Bawab et al. (El Bawab et al., J. Biol. Chem. 275:21508-21513 (2000), which is hereby incorporated by reference in its entirety) have shown previously that the human neutral ceramidase is localized in the mitochondria.

Alkaline ceramidases have optimal activity at a pH of 9-14. Two alkaline ceramidases were purified from Guinea pig skin epidermis. These two enzymes were membrane bound, and their estimated molecular masses on SDS-PAGE were 60 and 148 kDa, respectively (Yada et al., “Purification and Biochemical Characterization of Membrane-Bound Epidermal Ceramidases from Guinea Pig Skin,” J. Biol. Chem. 270:12677-12684 (1995), which is hereby incorporated by reference in its entirety). Two yeast (S. cerevisiae) alkaline ceramidases, phytoceramidase (YPC1p) and dihydroceramidase (YDC1p), were cloned and partially characterized by Mao et al., “Cloning of an Alkaline Ceramidase from Saccharomyces Cerevisiae. An Enzyme with Reverse (CoA-independent) Ceramide Synthase Activity,” J. Biol. Chem. 275:6876-6884 (2000), Mao et al., “Cloning and Characterization of a Saccharomyces Cerevisiae Alkaline Ceramidase with Specificity for Dihydroceramide,” J. Biol. Chem. 275:31369-31378 (2000), which are hereby incorporated by reference in their entirety. YPC1p was cloned as a high copy suppressor of the growth inhibition of FB1 as it has fumonisin resistant ceramide synthase activity. The second alkaline ceramidase, YDC1p was identified by sequence homology to YPC1p. A database search reveals that YPC1p and YDC1p are not homologous to any proteins with known functions, but are homologous to putative proteins from Arabidoposis, C. elegans, peptides deduced from EST sequences of human, mouse, pig, zebra fish, and human genomic sequences. A human homologue has been identified and its cDNA has been cloned. Preliminary results show that this human homologue is also an alkaline ceramidase that selectively hydrolyzes phytoCer.

Ceramidase expression according to the present disclosure relates to the protein level and to expression of the gene encoding the enzyme. Ceramidase activity according to the present disclosure involves physical and chemical transformation of the enzyme on a particular substrate, as well as the effect of such transformation on target cells or tissue. Ceramidase expression can relate to the translation of the ceramidase enzyme from a sequence of DNA or RNA that encodes the protein sequence of the enzyme. A vector may drive expression of AC by transfecting a cell with a polynucleotide encoding the AC and including response elements that promote expression of the encoded product.

Ceramidase, for example, acid ceramidase (AC), is required to hydrolyze ceramide into sphingosine and free fatty acids. Sphingosine is rapidly converted to sphingosine-1-phosphate (S1P), another important signaling lipid that counteracts the effects of ceramide and promotes cell survival. Thus, AC acts as a “rheostat” that regulates the levels of ceramide and S1P in cells, and as such participates in the complex and delicate balance between death and survival.

Acid ceramidase (“AC”) as described herein is an enzyme that catalyzes the hydrolysis of ceramide to sphingosine and free fatty acid (Bernardo et al., “Purification, Characterization, and Biosynthesis of Human Acid Ceramidase,” J. Biol. Chem. 270(19):11098-102 (1995), which is hereby incorporated by reference in its entirety). Mature AC is a ^{˜}50 kDa protein composed of an α-subunit (^{˜}13 kDa) and a β-subunit (^{˜}40 kDa) (Bernardo et al., “Purification, Characterization, and Biosynthesis of Human Acid Ceramidase,” J. Biol. Chem. 270(19):11098-102 (1995), which is hereby incorporated by reference in its entirety). It is produced through cleavage of the AC precursor protein (Ferlinz et al., “Human Acid Ceramidase: Processing, Glycosylation, and Lysosomal Targeting,” J. Biol. Chem. 276(38):35352-60 (2001), which is hereby incorporated by reference in its entirety), which is the product of the AsahI gene (NCBI UniGene GeneID No. 427, which is hereby incorporated by reference in its entirety). The present disclosure demonstrates that ceramidase (e.g., AC) promotes repair of damaged fluid, organ, tissue, and/or cell and that ceramidase (e.g., AC) reduces and/or prevents inflammation.

Ceramidase is an enzyme that cleaves fatty acids from ceramide, producing sphingosine (SPH), which in turn is phosphorylated by a sphingosine kinase to form sphingosine-1-phosphate (S1P). Ceramidase is the only enzyme that can regulate ceramide hydrolysis to prevent cell death and SHPK is the only enzyme that can synthesize sphingosine 1 phosphate (S1P) from sphingosine (the ceramide hydrolysis product) to initiate cell survival. S1PR, a G protein-coupled receptor binds the lipid-signaling molecule S1P to induce cell proliferation, survival, and transcriptional activation. CERK is an phosphatase that phosphorylates ceramide into ceramide 1 phosphate to induce cell survival.

Presently, 7 human ceramidases encoded by 7 distinct genes have been cloned: (1) acid ceramidase (ASAH1)—associated with cell survival; (2) neutral ceramidase (ASAH2, ASAH2B, ASAH2C)—protective against inflammatory cytokines; (3) alkaline ceramidase 1 (ACER1)—mediating cell differentiation by controlling the generation of SPH and S1P; (4) alkaline ceramidase 2 (ACER2)—important for cell proliferation and survival; and (5) alkaline ceramidase 3 (ACER3).

The nucleotide sequences for nucleic acids encoding these ceramidases are shown in Table 2.

An adeno-associated viral vector as described herein may include any platform for gene delivery for the treatment or prevention of a condition, disease, or disorder. In one embodiment, Anc80, a relatively nascent technology, has shown considerable potential as a delivery vehicle for gene therapy in disease, for example, cardiac disease, hearing loss, vision loss and neurodegenerative diseases. Anc80 as an engineered gene therapy vector is synthetic in nature and is not known to circulate in humans. It has been shown to have reduced cross-reactivity with commonly used AAV vectors. Anc80, therefore, is a potent gene therapy vector, which is less likely to be recognized immunologically by antibodies against naturally occurring AAVs.

An Anc80 vector encoding acid ceramidase (Anc80.AC) has multiple advantages over other potential anti-apoptotic factors. The first advantage is that Anc80 has low or no toxicity. The AC protein, by itself, is not toxic. Physiological enzymes are not expected to have toxic effects. The biological function of AC is the control of ceramide metabolism has no direct influence other cellular signaling. Treated cells present only a modest increase in AC generation in cells post gene therapy treatment. The AC protein level expressed after treatment is far below extraordinarily high levels reported in aberrant diseased cells with poorly understood mechanisms. The AC protein exists in two forms and undergoes a transformation from an inactive to active form in the cell. The inactive AC precursor undergoes an auto-self cleavage to the active enzyme, which is responsible for hydrolyzing ceramide to sphingosine. This exquisitely evolved self-regulating mechanism, call the Sphingolipid Rheostat, regulates, by hydrolysis toxic levels of ceramides in the cell after exposure to stress. The transfection of cells with Anc80.AC can increase the cellular reservoir of inactive precursor, thereby allowing physiological sphingolipid levels to regulate the conversion to the active AC enzyme necessary for cellular robustness and organism survival. In addition, a mouse model has been created that is constantly overexpressing the AC enzyme (COEAC) in all tissues. The COEAC mice viability provides evidence that AC is a non-toxic protein.

As mentioned, Anc80, an engineered gene therapy vector, is synthetic in nature and shown to reduce cross-reactivity with commonly used AAV vectors. Anc80 is a potent gene therapy vector that is not known to circulate in humans, making it less likely to be recognized immunologically by antibodies against naturally occurring AAVs. Recently, it has been shown successful, robust, transfection of Anc80 virus into liver, eye and ear tissue in vivo (see Trayssac et al., “Role of Sphingolipids in Senescence: Implication in Aging and Age-Related Diseases,” J. Clin. Inves. 128(7):2702-2712 (2018), which is hereby incorporated by reference.)

In an embodiment, an adeno-associated viral vector is selected from an Anc80, such as Anc80L65, or AAV6, AAV1, AAV2, AAV8, AAV9, or any combination thereof. Table 1 contains sequences of adeno-associated viral vector plasmids used in accordance with the methods described herein.

TABLE 1
Sequences of Adeno-Associated Viral Vector Plasmids

| | |
|---|---|
| Anc80 | CTGCGCGCTCGCTCGCTCACTGAGGCCGCCCGGGCAAAGCCCGGGCGT |
| | CGGGCGACCTTTGGTCGCCCGGCCTCAGTGAGCGAGCGAGCGCGCAGA |
| | GAGGGAGTGGCCAACTCCATCACTAGGGGTTCCTTGTAGTTAATGATTA |
| | ACCCGCCATGCTACTTATCTACGTAGCCATGCTCTAGGAAGATCGGAAT |
| | TCGCCCTTAAGCTAGCTAGTTATTAATAGTAATCAATTACGGGGTCATT |
| | AGTTCATAGCCCATATATGGAGTTCCGCGTTACATAACTTACGGTAAAT |
| | GGCCCGCCTGGCTGACCGCCCAACGACCCCCGCCCATTGACGTCAATAA |
| | TGACGTATGTTCCCATAGTAACGCCAATAGGGACTTTCCATTGACGTCA |
| | ATGGGTGGAGTATTTACGGTAAACTGCCCACTTGGCAGTACATCAAGTG |
| | TATCATATGCCAAGTACGCCCCCTATTGACGTCAATGACGGTAAATGGC |
| | CCGCCTGGCATTATGCCCAGTACATGACCTTATGGGACTTTCCTACTTG |
| | GCAGTACATCTACGTATTAGTCATCGCTATTACCATGGTGATGCGGTTT |
| | TGGCAGTACATCAATGGGCGTGGATAGCGGTTTGACTCACGGGGATTTC |
| | CAAGTCTCCACCCCATTGACGTCAATGGGAGTTTGTTTTGGCACCAAAA |
| | TCAACGGGACTTTCCAAAATGTCGTAACAACTCCGCCCCATTGACGCAA |
| | ATGGGCGGTAGGCGTGTACGGTGGGAGGTCTATATAAGCAGAGCTGGT |
| | TTAGTGAACCGTCAGATCCTGCAGAAGTTGGTCGTGAGGCACTGGGCA |
| | GGTAAGTATCAAGGTTACAAGACAGGTTTAAGGAGACCAATAGAAACT |
| | GGGCTTGTCGAGACAGAGAAGACTCTTGCGTTTCTGATAGGCACCTATT |
| | GGTCTTACTGACATCCACTTTGCCTTTCTCTCCACAGGTGTCCAGGCGGC |
| | CGCNNNGGATCCAATCAACCTCTGGATTACAAAATTTGTGAAAGATTG |
| | ACTGGTATTCTTAACTATGTTGCTCCTTTTACGCTATGTGGATACGCTGC |
| | TTTAATGCCTTTGTATCATGCTATTGCTTCCCGTATGGCTTTCATTTTCTC |
| | CTCCTTGTATAAATCCTGGTTGCTGTCTCTTTATGAGGAGTTGTGGCCCG |
| | TTGTCAGGCAACGTGGCGTGGTGTGCACTGTGTTTGCTGACGCAACCCC |
| | CACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGC |
| | CCGCTGCTGGACAGGGGCTCGGCTGTTGGGCACTGACAATTCCGTGGTG |
| | TTGTCGGGGAAATCATCGTCCTTTCCTTGGCTGCTCGCCTGTGTTGCCAC |
| | CTGGATTCTGCGCGGGACGTCCTTCTGCTACGTCCCTTCGGCCCTCAATC |
| | CAGCGGACCTTCCTTCCCGCGGCCTGCTGCCGGCTCTGCGGCCTCTTCC |
| | GCGTCTTCGAGATCTGCCTCGACTGTGCCTTCTAGTTGCCAGCCATCTGT |
| | TGTTTGCCCCTCCCCCGTGCCTTCCTTGACCCTGGAAGGTGCCACTCCCA |
| | CTGTCCTTTCCTAATAAAATGAGGAAATTGCATCGCATTGTCTGAGTAG |
| | GTGTCATTCTATTCTGGGGGGTGGGGTGGGGCAGGACAGCAAGGGGGA |
| | GGATTGGGAAGACAATAGCAGGCATGCTGGGGACTCGAGTTAAGGGCG |
| | AATTCCCGATAAGGATCTTCCTAGAGCATGGCTACGTAGATAAGTAGCA |
| | TGGCGGGTTAATCATTAACTACAAGGAACCCCTAGTGATGGAGTTGGCC |
| | ACTCCCTCTCTGCGCGCTCGCTCGCTCACTGAGGCCGGGCGACCAAAGG |
| | TCGCCCGACGCCCGGGCTTTGCCCGGGCGGCCTCAGTGAGCGAGCGAG |
| | CGCGCAGCCTTAATTAACCTAATTCACTGGCCGTCGTTTTACAACGTCG |
| | TGACTGGGAAAACCCTGGCGTTACCCAACTTAATCGCCTTGCAGCACAT |
| | CCCCCTTTCGCCAGCTGGCGTAATAGCGAAGAGGCCCGCACCGATCGCC |
| | CTTCCCAACAGTTGCGCAGCCTGAATGGCGAATGGGACGCGCCCTGTA |
| | GCGGCGCATTAAGCGCGGCGGGTGTGGTGGTTACGCGCAGCGTGACCG |
| | CTACACTTGCCAGCGCCCTAGCGCCCGCTCCTTTCGCTTTCTTCCCTTCC |
| | TTTCTCGCCACGTTCGCCGGCTTTCCCCGTCAAGCTCTAAATCGGGGGC |
| | TCCCTTTAGGGTTCCGATTTAGTGCTTTACGGCACCTCGACCCCAAAAA |
| | ACTTGATTAGGGTGATGGTTCACGTAGTGGGCCATCGCCCTGATAGACG |
| | GTTTTTCGCCCTTTGACGTTGGAGTCCACGTTCTTTAATAGTGGACTCTT |
| | GTTCCAAACTGGAACAACACTCAACCCTATCTCGGTCTATTCTTTTGATT |
| | TATAAGGGATTTTGCCGATTTCGGCCTATTGGTTAAAAAATGAGCTGAT |
| | TTAACAAAAATTTAACGCGAATTTTAACAAAATATTAACGTTTATAATT |
| | TCAGGTGGCATCTTTCGGGGAAATGTGCGCGGAACCCCTATTTGTTTAT |
| | TTTTCTAAATACATTCAAATATGTATCCGCTCATGAGACAATAACCCTG |
| | ATAAATGCTTCAATAATATTGAAAAAGGAAGAGTATGAGTATTCAACA |
| | TTTCCGTGTCGCCCTTATTCCCTTTTTTGCGGCATTTTGCCTTCCTGTTTT |
| | TGCTCACCCAGAAACGCTGGTGAAAGTAAAAGATGCTGAAGATCAGTT |
| | GGGTGCACGAGTGGGTTACATCGAACTGGATCTCAATAGTGGTAAGAT |
| | CCTTGAGAGTTTTCGCCCCGAAGAACGTTTTCCAATGATGAGCACTTTT |
| | AAAGTTCTGCTATGTGGCGCGGTATTATCCCGTATTGACGCCGGGCAAG |
| | AGCAACTCGGTCGCCGCATACACTATTCTCAGAATGACTTGGTTGAGTA |
| | CTCACCAGTCACAGAAAAGCATCTTACGGATGGCATGACAGTAAGAGA |
| | ATTATGCAGTGCTGCCATAACCATGAGTGATAACACTGCGGCCAACTTA |
| | CTTCTGACAACGATCGGAGGACCGAAGGAGCTAACCGCTTTTTTGCACA |
| | ACATGGGGGATCATGTAACTCGCCTTGATCGTTGGGAACCGGAGCTGA |
| | ATGAAGCCATACCAAACGACGAGCGTGACACCACGATGCCTGTAGTAA |
| | TGGTAACAACGTTGCGCAAACTATTAACTGGCGAACTACTTACTCTAGC |
| | TTCCCGGCAACAATTAATAGACTGGATGGAGGCGGATAAAGTTGCAGG |
| | ACCACTTCTGCGCTCGGCCCTTCCGGCTGGCTGGTTTATTGCTGATAAAT |
| | CTGGAGCCGGTGAGCGTGGGTCTCGCGGTATCATTGCAGCACTGGGGC |
| | CAGATGGTAAGCCCTCCCGTATCGTAGTTATCTACACGACGGGGAGTCA |
| | GGCAACTATGGATGAACGAAATAGACAGATCGCTGAGATAGGTGCCTC |
| | ACTGATTAAGCATTGGTAACTGTCAGACCAAGTTTACTCATATATACTT |
| | TAGATTGATTTAAAACTTCATTTTTAATTTAAAAGGATCTAGGTGAAGA |
| | TCCTTTTTGATAATCTCATGACCAAAATCCCTTAACGTGAGTTTTCGTTC |
| | CACTGAGCGTCAGACCCCGTAGAAAAGATCAAAGGATCTTCTTGAGAT |
| | CCTTTTTTTCTGCGCGTAATCTGCTGCTTGCAAACAAAAAAACCACCGC |
| | TACCAGCGGTGGTTTGTTTGCCGGATCAAGAGCTACCAACTCTTTTTCC |
| | GAAGGTAACTGGCTTCAGCAGAGCGCAGATACCAAATACTGTCCTTCTA |
| | GTGTAGCCGTAGTTAGGCCACCACTTCAAGAACTCTGTAGCACCGCCTA |
| | CATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAG |
| | GCGCAGCGGTCGGGCTGAACGGGGGGTTCGTGCACACAGCCCAGCTTG |
| | GAGCGAACGACCTACACCGAACTGAGATACCTACAGCGTGAGCTATGA |
| | GAAAGCGCCACGCTTCCCGAAGGGAGAAAGGCGGACAGGTATCCGGTA |
| | AGCGGCAGGGTCGGAACAGGAGAGCGCACGAGGGAGCTTCCAGGGGG |
| | AAACGCCTGGTATCTTTATAGTCCTGTCGGGTTTCGCCACCTCTGACTTG |
| | AGCGTCGATTTTTGTGATGCTCGTCAGGGGGGCGGAGCCTATGGAAAA |
| | ACGCCAGCAACGCGGCCTTTTTACGGTTCCTGGCCTTTTGCTGCGGTTTT |
| | GCTCACATGTTCTTTCCTGCGTTATCCCCTGATTCTGTGGATAACCGTAT |
| | TACCGCCTTTGAGTGAGCTGATACCGCTCGCCGCAGCCGAACGACCGA |
| | GCGCAGCGAGTCAGTGAGCGAGGAAGCGGAAGAGCGCCCAATACGCA |
| | AACCGCCTCTCCCCGCGCGTTGGCCGATTCATTAATGCAGCTGGCACGA |
| | CAGGTTTCCCGACTGGAAAGCGGGCAGTGAGCGCAACGCAATTAATGT |
| | GAGTTAGCTCACTCATTAGGCACCCCAGGCTTTACACTTTATGCTTCCG |
| | GCTCGTATGTTGTGTGGAATTGTGAGCGGATAACAATTTCACACAGGAA |
| | ACAGCTATGACCATGATTACGCCAGATTTAATTAAGG (SEQ ID NO: 1) |
| Anc80L65 | AGCTTCGATCAACTACGCAGACAGGTACCAAAACAAATGTTCTCGTCAC |
| | GTGGGCATGAATCTGATGCTGTTTCCCTGCAGACAATGCGAGAGAATG |
| | AATCAGAATTCAAATATCTGCTTCACTCACGGACAGAAAGACTGTTTAG |
| | AGTGCTTTCCCGTGTCAGAATCTCAACCCGTTTCTGTCGTCAAAAAGGC |
| | GTATCAGAAACTGTGCTACATTCATCATATCATGGGAAAGGTGCCAGAC |
| | GCTTGCACTGCCTGCGATCTGGTCAATGTGGATTTGGATGACTGCATCT |
| | TTGAACAATAAATGATTTAAATCAGGTATGGCTGCCGATGGTTATCTTC |
| | CAGATTGGCTCGAGGACAACCTCTCTGAGGGCATTCGCGAGTGGTGGG |
| | ACTTGAAACCTGGAGCCCCGAAACCCAAAGCCAACCAGCAAAAGCAGG |
| | ACGACGGCCGGGGTCTGGTGCTTCCTGGCTACAAGTACCTCGGACCCTT |
| | CAACGGACTCGACAAGGGGGAGCCCGTCAACGCGGCGGACGCAGCGG |
| | CCCTCGAGCACGACAAGGCCTACGACCAGCAGCTCAAAGCGGGTGACA |
| | ATCCGTACCTGCGGTATAACCACGCCGACGCCGAGTTTCAGGAGCGTCT |
| | GCAAGAAGATACGTCTTTTGGGGGCAACCTCGGGCGAGCAGTCTTCCA |
| | GGCCAAGAAGCGGGTTCTCGAACCTCTCGGTCTGGTTGAGGAAGGCGC |
| | TAAGACGGCTCCTGGAAAGAAGAGGCCGGTAGAGCAATCACCCCAGGA |
| | ACCAGACTCCTCTTCGGGCATCGGCAAGAAAGGCCAGCAGCCCGCGAG |
| | AAAGAGACTCAACTTTGGGCAGACAGGCGACTCAGAGTCAGTGCCCGA |
| | CCCTCAACCACTCGGAGAACCCCCCGCAGCCCCCTCTGGTGTGGGATCT |
| | AATACAATGGCTGCAGGCGGTGGCGCTCCAATGGCAGACAATAACGAA |
| | GGCGCCGACGGAGTGGGTAACGCCTCAGGAAATTGGCATTGCGATTCC |
| | ACATGGCTGGGCGACAGAGTCATCACCACCAGCACCCGAACCTGGGCC |
| | CTCCCCACCTACAACAACCACCTCTACAAGCAAATCTCCAGCCAATCGG |
| | GAGGCAGCACCAACGACAACACCTACTTCGGCTACAGCACCCCCTGGG |
| | GGTATTTTGACTTTAACAGATTCCACTGCCACTTCTCACCACGTGACTG |
| | GCAGCGACTCATCAACAACAACTGGGGATTCCGGCCCAAGAAGCTCAA |
| | CTTCAAGCTCTTCAACATCCAGGTCAAGGAGGTCACGACGAATGATGG |
| | CACCACGACCATCGCCAATAACCTTACCAGCACGGTTCAGGTCTTTACG |
| | GACTCGGAATACCAGCTCCCGTACGTCCTCGGCTCTGCGCACCAGGGCT |
| | GCCTGCCTCCGTTCCCGGCGGACGTCTTCATGATTCCTCAGTACGGGTA |
| | CCTGACTCTGAACAATGGCAGTCAGGCCGTGGGCCGTTCCTCCTTCTAC |
| | TGCCTGGAGTACTTTCCTTCTCAAATGCTGAGAACGGGCAACAACTTTC |
| | AGTTCAGCTACACGTTTGAGGACGTGCCTTTTCACAGCAGCTACGCGCA |
| | CAGCCAAAGCCTGGACCGGCTGATGAACCCCCTCATCGACCAGTACCT |
| | GTACTACCTGTCTCGGACTCAGACCACGAGTGGTACCGCAGGAAATCG |
| | GACGTTGCAATTTTCTCAGGCCGGGCCTAGTAGCATGGCGAATCAGGCC |
| | AAAAACTGGCTACCCGGGCCCTGCTACCGGCAGCAACGCGTCTCCAAG |
| | ACAACCAATCAAAATAACAACAGCAACTTTGCCTGGACCGGTGCCACC |
| | AAGTATCATCTGAATGGCAGAGACTCTCTGGTAAATCCCGGTCCCGCTA |
| | TGGCAACCCACAAGGACGACGAAGACAAATTTTTTCCGATGAGCGGAG |
| | TCTTAATATTTGGGAAACAGGGAGCTGGAAATAGCAACGTGGACCTTG |
| | ACAACGTTATGATAACCAACGAGGAAGAAATTAAAACCACCAACCCAG |
| | TGGCCACAGAAGAGTACGGCACGGTGGCCACTAACCTGCAATCGGCCA |
| | ACACCGCTCCTGCTACAGGGACCGTCAACAGTCAAGGAGCCTTACCTG |
| | GCATGGTCTGGCAGGACCGGGACGTGTACCTGCAGGGTCCTATCTGGG |
| | CCAAGATTCCTCACACGGACGGACACTTTCATCCCTCGCCGCTGATGGG |
| | AGGCTTTGGACTGAAACACCCGCCTCCTCAGATCCTGATTAAGAATACA |
| | CCTGTTCCCGCGAATCCTCCAACTACCTTCAGTCCAGCTAAGTTTGCGTC |
| | GTTCATCACGCAGTACAGCACCGGACAGGTCAGCGTGGAAATTGAATG |
| | GGAGCTGCAGAAAGAAAACAGCAAACGCTGGAACCCAGAGATTCAAT |
| | ACACTTCCAACTACAACAAATCTACAAATGTGGACTTTGCTGTTGACAC |
| | AAATGGCGTTTATTCTGAGCCTCGCCCCATCGGCACCCGTTACCTCACC |
| | CGTAATCTGTAAACTAGTTTGCTTGTTAATCAATAAACCGTTTAATTCGT |
| | TTCAGTTGAACTTTGGTCTCTGCGTATTTCTTTCTTATCTAGTTTCCATGC |
| | TCTAGAGGTCCTGTATTAGAGGTCACGTGAGTGTTTTGCGACATTTTGC |
| | GACACCATGTGGTCACGCTGGGTATTTAAGCCCGAGTGAGCACGCAGG |
| | GTCTCCATTTTGAAGCGGGAGGTTTGAACGCGCAGCCGCCAAGCCGAA |
| | TTCTGCAGATATCCATCACACTGGCGGCCGCTCGACTAGAGCGGCCGCC |
| | ACCGCGGTGGAGCTCCAGCTTTTGTTCGCGCGCTTGGCGTAATCATGGT |
| | CATAGCTGTTTCCTGTGTGAAATTCCACAGCCTGGGGTGCCTAATTGCG |
| | TTGCGCTCACTGCCCGCTTTCCAGTCGGGAAACCTGTCGTGCCAGCTGC |
| | ATTAATGAATCGGCCAACGCGCGGGGAGAGGCGGTTTGCGTATTGGGC |
| | GCTCTTCCGCTTCCTCGCTCACTGACTCGCTGCGCTCGGTCGTTCGGCTG |
| | CGGCGAGCGGTATCAGCTCACTCAAAGGCGGTAATACGGTTATCCACA |
| | GAATCAGGGGATAACGCAGGAAAGAACATGTGAGCAAAAGGCCAGCA |
| | AAAGGCCAGGAACCGTAAAAAGGCCGCGTTGCTGGCGTTTTTCCATAG |
| | GCTCCGCCCCCCTGACGAGCATCACAAAAATCGACGCTCAAGTCAGAG |
| | GTGGCGAAACCCGACAGGACTATAAAGATACCAGGCGTTTCCCCCTGG |
| | AAGCTCCCTCGTGCGCTCTCCTGTTCCGACCCTGCCGCTTACCGGATAC |
| | CTGTCCGCCTTTCTCCCTTCGGGAAGCGTGGCGCTTTCTCATAGCTCACG |
| | CTGTAGGTATCTCAGTTCGGTGTAGGTCGTTCGCTCCAAGCTGGGCTGT |
| | GTGCACGAACCCCCCGTTCAGCCCGACCGCTGCGCCTTATCCGGTAACT |
| | ATCGTCTTGAGTCCAACCCGGTAAGACACGACTTATCGCCACTGGCAGC |
| | AGCCACTGGTAACAGGATTAGCAGAGCGAGGTATGTAGGCGGTGCTAC |
| | AGAGTTCTTGAAGTGGTGGCCTAACTACGGCTACACTAGAAGAACAGT |
| | ATTTGGTATCTGCGCTCTGCTGAAGCCAGTTACCTTCGGAAAAAGAGTT |
| | GGTAGCTCTTGATCCGGCAAACAAACCACCGCTGGTAGCGGTGGTTTTT |
| | TTGTTTGCAAGCAGCAGATTACGCGCAGAAAAAAAGGATCTCAAGAAG |
| | ATCCTTTGATCTTTTCTACGGGGTCTGACGCTCAGTGGAACGAAAACTC |
| | ACGTTAAGGGATTTTGGTCATGAGATTATCAAAAAGGATCTTCACCTAG |
| | ATCCTTTTAAATTAAAAATGAAGTTTTAAATCAATCTAAAGTATATATG |
| | AGTAAACTTGGTCTGACAGAAATAATAAAAAAGCCGGATTAATAATCT |
| | GGCTTTTTATATTCTCTCTCTAGTATATAAACGCAGAAAGGCCCACCCG |
| | AAGGTGAGCCAGTGTGACTCTAGTATTATTAGAAAAACTCATCGAGCAT |
| | CAAATGAAACTGCAATTTATTCATATCAGGATTATCAATACCATATTTT |
| | TGAAAAAGCCGTTTCTGTAATGAAGGAGAAAACTCACCGAGGCAGTTC |
| | CAAAGAATGGCAAGGTCCTGGTAACGGTCTGCGATTCCGACCCGTCCA |
| | ACATCAATACAACCTATTAATTTCCCCTCGTCAAAAATAAGGTTATCAA |
| | GTGAGAAATCACCATGAGTGACGACTGAATCCGGTGAGAATGGCAAGA |
| | GCTTGTGCATTTCTTTCCAGACTTGTTCAACAGGCCAGCCATTACGCTCG |
| | TCATCAAAATCACTCGCATCAACCAAACCGTTATTCATGCGTGATTGCG |
| | CCTGAGCAAGACGAAATACACGATCGCTGTTAAAAGGACAATTACAAA |
| | CAGGAATCGAATGTAACCGGCGCAGGAACACGGCCAGCGCATCAACAA |
| | TATTTTCACCTGAATCAGGATATTCTTCTAATACCTGGAAGGCTGTTTTC |
| | CCAGGAATCGCGGTGGTGAGTAACCACGCATCATCAGGAGTACGGATA |
| | AAATGCTTGATGGTCGGGAGAGGCATAAACTCCGTCAGCCAGTTGAGA |
| | CGGACCATCTCATCTGTAACATCATTGGCAACGCTACCTTTGCCATGTTT |
| | CAGAAACAACTCTGGCGCATCGGGCTTCCCATACAAGCGATAGATTGTC |
| | GCACCTGATTGCCCGACATTATCGCGAGCCCATTTATACCCATATAAAT |
| | CAGCGTCCATGTTGGAGTTTAAGCGCGGACGGGAGCAAGACGTTTCCC |
| | GTTGAATATGGCTCATAACACCCCTTGTATTACTGTTTATGTAAGCAGA |
| | CAGTTTTATTGTTCATGATGATATATTTTTATCTTGTGCAATGTAACATC |
| | AGAGATTTTGAGACACAACGTGGCTTTGTTGAATAAATCGAACTTTTGC |
| | TGAGTTGAAGGATCAGCTCTAGTAAAATAATAAAAAAGCCGGATTAAT |
| | AATCTGGCTTTTTATATTCTCTCTCTAGTATATAAACGCAGAAAGGCCC |
| | ACCCGAAGGTGAGCCAGTGTGACGGCACATTTCCCCGAAAAGTGCCAC |
| | CTAAATTGTAAGCGTTAATATTTTGTTAAAATTCGCGTTAAATTTTTGTT |
| | AAATCAGCTCATTTTTTAACCAATAGGCCGAAATCGGCAAAATCCCTTA |
| | TAAATCAAAAGAATAGACCGAGATAGGGTTGAGTGTTGTTCCAGTTTG |
| | GAACAAGAGTCCACTATTAAAGAACGTGGACTCCAACGTCAAAGGGCG |
| | AAAAACCGTCTATCAGGGCGATGGCCCACTACGTGAACCATCACCCTA |
| | ATCAAGTTTTTTGGGGTCGAGGTGCCGTAAAGCACTAAATCGGAACCCT |
| | AAAGGGAGCCCCCGATTTAGAGCTTGACGGGGAAAGCCGGCGAACGTG |
| | GCGAGAAAGGAAGGGAAGAAAGCGAAAGGAGCGGGCGCTAGGGCGCT |
| | GGCAAGTGTAGCGGTCACGCTGCGCGTAACCACCACACCCGCCGCGCT |
| | TAATGCGCCGCTACAGGGCGCGTCCCATTCGCCATTCAGGCTGCGCAAC |
| | TGTTGGGAAGGGCGATCGGTGCGGGCCTCTTCGCTATTACGCCAGCTGG |
| | CGAAAGGGGGATGTGCTGCAAGGCGATTAAGTTGGGTAACGCCAGGGT |
| | TTTCCCAGTCACGACGTTGTAAAACGACGGCCAGTGAGCGCGCGGCGA |
| | ATTGGGTACCGGGCCCCCCCTCGAGGTCGACGGTATCGTATTTAAGCCC |
| | GAGTGAGCACGCAGGGTCTCCATTTTGAAGCGGGAGGTTTGAACGCGC |
| | AGCCGCCATGCCGGGGTTTTACGAGATTGTGATTAAGGTCCCCAGCGAC |
| | CTTGACGAGCATCTGCCCGGCATTTCTGACAGCTTTGTGAACTGGGTGG |
| | CCGAGAAGGAATGGGAGTTGCCGCCAGATTCTGACATGGATCTGAATC |
| | TGATTGAGCAGGCACCCCTGACCGTGGCCGAGAAGCTGCAGCGCGACT |
| | TTCTGACGGAATGGCGCCGTGTGAGTAAGGCCCCGGAGGCTCTTTTCTT |
| | TGTGCAATTTGAGAAGGGAGAGAGCTACTTCCACATGCACGTGCTCGTG |
| | GAAACCACCGGGGTGAAATCCATGGTTTTGGGACGTTTCCTGAGTCAGA |
| | TTCGCGAAAAACTGATTCAGAGAATTTACCGCGGGATCGAGCCGACTTT |
| | GCCAAACTGGTTCGCGGTCACAAAGACCAGAAATGGCGCCGGAGGCGG |
| | GAACAAGGTGGTGGATGAGTGCTACATCCCCAATTACTTGCTCCCCAAA |
| | ACCCAGCCTGAGCTCCAGTGGGCGTGGACTAATATGGAACAGTATTTA |
| | AGCGCCTGTTTGAATCTCACGGAGCGTAAACGGTTGGTGGCGCAGCATC |
| | TGACGCACGTGTCGCAGACGCAGGAGCAGAACAAAGAGAATCAGAATC |
| | CCAATTCTGATGCGCCGGTGATCAGATCAAAAACTTCAGCCAGGTACAT |
| | GGAGCTGGTCGGGTGGCTCGTGGACAAGGGGATTACCTCGGAGAAGCA |
| | GTGGATCCAGGAGGACCAGGCCTCATACATCTCCTTCAATGCGGCCTCC |
| | AACTCGCGGTCCCAAATCAAGGCTGCCTTGGACAATGCGGGAAAGATT |
| | ATGAGCCTGACTAAAACCGCCCCCGACTACCTGGTGGGCCAGCAGCCC |
| | GTGGAGGACATTTCCAGCAATCGGATTTATAAAATTTTGGAACTAAACG |
| | GGTACGATCCCCAATATGCGGCTTCCGTCTTTCTGGGATGGGCCACGAA |
| | AAAGTTCGGCAAGAGGAACACCATCTGGCTGTTTGGGCCTGCAACTAC |
| | CGGGAAGACCAACATCGCGGAGGCCATAGCCCACACTGTGCCCTTCTA |
| | CGGGTGCGTAAACTGGACCAATGAGAACTTTCCCTTCAACGACTGTGTC |
| | GACAAGATGGTGATCTGGTGGGAGGAGGGGAAGATGACCGCCAAGGTC |
| | GTGGAGTCGGCCAAAGCCATTCTCGGAGGAAGCAAGGTGCGCGTGGAC |
| | CAGAAATGCAAGTCCTCGGCCCAGATAGACCCGACTCCCGTGATCGTC |
| | ACCTCCAACACCAACATGTGCGCCGTGATTGACGGGAACTCAACGACC |
| | TTCGAACACCAGCAGCCGTTGCAAGACCGGATGTTCAAATTTGAACTCA |
| | CCCGCCGTCTGGATCATGACTTTGGGAAGGTCACCAAGCAGGAAGTCA |
| | AAGACTTTTTCCGGTGGGCAAAGGATCACGTGGTTGAGGTGGAGCATG |
| | AATTCTACGTCAAAAAGGGTGGAGCCAAGAAAAGACCCGCCCCCAGTG |
| | ACGCAGATATAAGTGAGCCCAAACGGGTGCGCGAGTCAGTTGCGCAGC |
| | CATCGACGTCAGACGCGGA (SEQ ID NO: 2) |
| AAV1 | GCTGCGCAACTGTTGGGAAGGGCGATCGGTGCGGGCCTCTTCGCTATTA |
| | CGCCAGCTGGCGAAAGGGGGATGTGCTGCAAGGCGATTAAGTTGGGTA |
| | ACGCCAGGGTTTTCCCAGTCACGACGTTGTAAAACGACGGCCAGTGAG |
| | CGCGCGTAATACGACTCACTATAGGGCGAATTGGGTACCGGGCCCCCC |
| | CTCGAGGTCGACGGTATCGGGGGAGCTCGCAGGGTCTCCATTTTGAAGC |
| | GGGAGGTTTGAACGCGCAGCCGCCATGCCGGGGTTTTACGAGATTGTG |
| | ATTAAGGTCCCCAGCGACCTTGACGAGCATCTGCCCGGCATTTCTGACA |
| | GCTTTGTGAACTGGGTGGCCGAGAAGGAATGGGAGTTGCCGCCAGATT |
| | CTGACATGGATCTGAATCTGATTGAGCAGGCACCCCTGACCGTGGCCGA |
| | GAAGCTGCAGCGCGACTTTCTGACGGAATGGCGCCGTGTGAGTAAGGC |
| | CCCGGAGGCTCTTTTCTTTGTGCAATTTGAGAAGGGAGAGAGCTACTTC |
| | CACATGCACGTGCTCGTGGAAACCACCGGGGTGAAATCCATGGTTTTGG |
| | GACGTTTCCTGAGTCAGATTCGCGAAAAACTGATTCAGAGAATTTACCG |
| | CGGGATCGAGCCGACTTTGCCAAACTGGTTCGCGGTCACAAAGACCAG |
| | AAATGGCGCCGGAGGCGGGAACAAGGTGGTGGATGAGTGCTACATCCC |
| | CAATTACTTGCTCCCCAAAACCCAGCCTGAGCTCCAGTGGGCGTGGACT |
| | AATATGGAACAGTATTTAAGCGCCTGTTTGAATCTCACGGAGCGTAAAC |
| | GGTTGGTGGCGCAGCATCTGACGCACGTGTCGCAGACGCAGGAGCAGA |
| | ACAAAGAGAATCAGAATCCCAATTCTGATGCGCCGGTGATCAGATCAA |
| | AAACTTCAGCCAGGTACATGGAGCTGGTCGGGTGGCTCGTGGACAAGG |
| | GGATTACCTCGGAGAAGCAGTGGATCCAGGAGGACCAGGCCTCATACA |
| | TCTCCTTCAATGCGGCCTCCAACTCGCGGTCCCAAATCAAGGCTGCCTT |
| | GGACAATGCGGGAAAGATTATGAGCCTGACTAAAACCGCCCCCGACTA |
| | CCTGGTGGGCCAGCAGCCCGTGGAGGACATTTCCAGCAATCGGATTTAT |
| | AAAATTTTGGAACTAAACGGGTACGATCCCCAATATGCGGCTTCCGTCT |
| | TTCTGGGATGGGCCACGAAAAAGTTCGGCAAGAGGAACACCATCTGGC |
| | TGTTTGGGCCTGCAACTACCGGGAAGACCAACATCGCGGAGGCCATAG |
| | CCCACACTGTGCCCTTCTACGGGTGCGTAAACTGGACCAATGAGAACTT |
| | TCCCTTCAACGACTGTGTCGACAAGATGGTGATCTGGTGGGAGGAGGG |
| | GAAGATGACCGCCAAGGTCGTGGAGTCGGCCAAAGCCATTCTCGGAGG |
| | AAGCAAGGTGCGCGTGGACCAGAAATGCAAGTCCTCGGCCCAGATAGA |
| | CCCGACTCCCGTGATCGTCACCTCCAACACCAACATGTGCGCCGTGATT |
| | GACGGGAACTCAACGACCTTCGAACACCAGCAGCCGTTGCAAGACCGG |
| | ATGTTCAAATTTGAACTCACCCGCCGTCTGGATCATGACTTTGGGAAGG |
| | TCACCAAGCAGGAAGTCAAAGACTTTTTCCGGTGGGCAAAGGATCACG |
| | TGGTTGAGGTGGAGCATGAATTCTACGTCAAAAAGGGTGGAGCCAAGA |
| | AAAGACCCGCCCCCAGTGACGCAGATATAAGTGAGCCCAAACGGGTGC |
| | GCGAGTCAGTTGCGCAGCCATCGACGTCAGACGCGGAAGCTTCGATCA |
| | ACTACGCAGACAGGTACCAAAACAAATGTTCTCGTCACGTGGGCATGA |
| | ATCTGATGCTGTTTCCCTGCAGACAATGCGAGAGAATGAATCAGAATTC |
| | AAATATCTGCTTCACTCACGGACAGAAAGACTGTTTAGAGTGCTTTCCC |
| | GTGTCAGAATCTCAACCCGTTTCTGTCGTCAAAAAGGCGTATCAGAAAC |
| | TGTGCTACATTCATCATATCATGGGAAAGGTGCCAGACGCTTGCACTGC |
| | CTGCGATCTGGTCAATGTGGATTTGGATGACTGCATCTTTGAACAATAA |
| | ATGATTTAAATCAGGTATGGCTGCCGATGGTTATCTTCCAGATTGGCTC |
| | GAGGACAACCTCTCTGAGGGCATTCGCGAGTGGTGGGACTTGAAACCT |
| | GGAGCCCCGAAGCCCAAAGCCAACCAGCAAAAGCAGGACGACGGCCG |
| | GGGTCTGGTGCTTCCTGGCTACAAGTACCTCGGACCCTTCAACGGACTC |
| | GACAAGGGGGAGCCCGTCAACGCGGCGGACGCAGCGGCCCTCGAGCAC |
| | GACAAGGCCTACGACCAGCAGCTCAAAGCGGGTGACAATCCGTACCTG |
| | CGGTATAACCACGCCGACGCCGAGTTTCAGGAGCGTCTGCAAGAAGAT |
| | ACGTCTTTTGGGGGCAACCTCGGGCGAGCAGTCTTCCAGGCCAAGAAG |
| | CGGGTTCTCGAACCTCTCGGTCTGGTTGAGGAAGGCGCTAAGACGGCTC |
| | CTGGAAAGAAACGTCCGGTAGAGCAGTCGCCACAAGAGCCAGACTCCT |
| | CCTCGGGCATCGGCAAGACAGGCCAGCAGCCCGCTAAAAAGAGACTCA |
| | ATTTTGGTCAGACTGGCGACTCAGAGTCAGTCCCCGATCCACAACCTCT |
| | CGGAGAACCTCCAGCAACCCCCGCTGCTGTGGGACCTACTACAATGGCT |
| | TCAGGCGGTGGCGCACCAATGGCAGACAATAACGAAGGCGCCGACGGA |
| | GTGGGTAATGCCTCAGGAAATTGGCATTGCGATTCCACATGGCTGGGCG |
| | ACAGAGTCATCACCACCAGCACCCGCACCTGGGCCTTGCCCACCTACAA |
| | TAACCACCTCTACAAGCAAATCTCCAGTGCTTCAACGGGGGCCAGCAA |
| | CGACAACCACTACTTCGGCTACAGCACCCCCTGGGGGTATTTTGATTTC |
| | AACAGATTCCACTGCCACTTTTCACCACGTGACTGGCAGCGACTCATCA |
| | ACAACAATTGGGGATTCCGGCCCAAGAGACTCAACTTCAAACTCTTCAA |
| | CATCCAAGTCAAGGAGGTCACGACGAATGATGGCGTCACAACCATCGC |
| | TAATAACCTTACCAGCACGGTTCAAGTCTTCTCGGACTCGGAGTACCAG |
| | CTTCCGTACGTCCTCGGCTCTGCGCACCAGGGCTGCCTCCCTCCGTTCCC |
| | GGCGGACGTGTTCATGATTCCGCAATACGGCTACCTGACGCTCAACAAT |
| | GGCAGCCAAGCCGTGGGACGTTCATCCTTTTACTGCCTGGAATATTTCC |
| | CTTCTCAGATGCTGAGAACGGGCAACAACTTTACCTTCAGCTACACCTT |
| | TGAGGAAGTGCCTTTCCACAGCAGCTACGCGCACAGCCAGAGCCTGGA |
| | CCGGCTGATGAATCCTCTCATCGACCAATACCTGTATTACCTGAACAGA |
| | ACTCAAAATCAGTCCGGAAGTGCCCAAAACAAGGACTTGCTGTTTAGC |
| | CGTGGGTCTCCAGCTGGCATGTCTGTTCAGCCCAAAAACTGGCTACCTG |
| | GACCCTGTTATCGGCAGCAGCGCGTTTCTAAAACAAAAACAGACAACA |
| | ACAACAGCAATTTTACCTGGACTGGTGCTTCAAAATATAACCTCAATGG |
| | GCGTGAATCCATCATCAACCCTGGCACTGCTATGGCCTCACACAAAGAC |
| | GACGAAGACAAGTTCTTTCCCATGAGCGGTGTCATGATTTTTGGAAAAG |
| | AGAGCGCCGGAGCTTCAAACACTGCATTGGACAATGTCATGATTACAG |
| | ACGAAGAGGAAATTAAAGCCACTAACCCTGTGGCCACCGAAAGATTTG |
| | GGACCGTGGCAGTCAATTTCCAGAGCAGCAGCACAGACCCTGCGACCG |
| | GAGATGTGCATGCTATGGGAGCATTACCTGGCATGGTGTGGCAAGATA |
| | GAGACGTGTACCTGCAGGGTCCCATTTGGGCCAAAATTCCTCACACAGA |
| | TGGACACTTTCACCCGTCTCCTCTTATGGGCGGCTTTGGACTCAAGAAC |
| | CCGCCTCCTCAGATCCTCATCAAAAACACGCCTGTTCCTGCGAATCCTC |
| | CGGCGGAGTTTTCAGCTACAAAGTTTGCTTCATTCATCACCCAATACTC |
| | CACAGGACAAGTGAGTGTGGAAATTGAATGGGAGCTGCAGAAAGAAA |
| | ACAGCAAGCGCTGGAATCCCGAAGTGCAGTACACATCCAATTATGCAA |
| | AATCTGCCAACGTTGATTTTACTGTGGACAACAATGGACTTTATACTGA |
| | GCCTCGCCCCATTGGCACCCGTTACCTTACCCGTCCCCTGTAATTACGTG |
| | TTAATCAATAAACCGGTTGATTCGTTTCAGTTGAACTTTGGTCTCCTGTC |
| | CTTCTTATCTTATCGGTTACCATGGTTATAGCTTACACATTAACTGCTTG |
| | GTTGCGCTTCGCGATAAAAGACTTACGTCATCGGGTTACCCCTAGGGGG |
| | ATCCACTAGTTCTAGAGGTCCTGTATTAGAGGTCACGTGAGTGTTTTGC |
| | GACATTTTGCGACACCATGTGGTCACGCTGGGTATTTAAGCCCGAGTGA |
| | GCACGCAGGGTCTCCATTTTGAAGCGGGAGGTTTGAACGCGCAGCCGC |
| | CAAGCCGAATTCTGCAGATATCCATCACACTGGCGGCCGCTCGACTAGA |
| | GCGGCCGCCACCGCGGTGGAGCTCCAGCTTTTGTTCCCTTTAGTGAGGG |
| | TTAATTGCGCGCTTGGCGTAATCATGGTCATAGCTGTTTCCTGTGTGAA |
| | ATTGTTATCCGCTCACAATTCCACACAACATACGAGCCGGAAGCATAAA |
| | GTGTAAAGCCTGGGGTGCCTAATGAGTGAGCTAACTCACATTAATTGCG |
| | TTGCGCTCACTGCCCGCTTTCCAGTCGGGAAACCTGTCGTGCCAGCTGC |
| | ATTAATGAATCGGCCAACGCGCGGGGAGAGGCGGTTTGCGTATTGGGC |
| | GCTCTTCCGCTTCCTCGCTCACTGACTCGCTGCGCTCGGTCGTTCGGCTG |
| | CGGCGAGCGGTATCAGCTCACTCAAAGGCGGTAATACGGTTATCCACA |
| | GAATCAGGGGATAACGCAGGAAAGAACATGTGAGCAAAAGGCCAGCA |
| | AAAGGCCAGGAACCGTAAAAAGGCCGCGTTGCTGGCGTTTTTCCATAG |
| | GCTCCGCCCCCCTGACGAGCATCACAAAAATCGACGCTCAAGTCAGAG |
| | GTGGCGAAACCCGACAGGACTATAAAGATACCAGGCGTTTCCCCCTGG |
| | AAGCTCCCTCGTGCGCTCTCCTGTTCCGACCCTGCCGCTTACCGGATAC |
| | CTGTCCGCCTTTCTCCCTTCGGGAAGCGTGGCGCTTTCTCATAGCTCACG |
| | CTGTAGGTATCTCAGTTCGGTGTAGGTCGTTCGCTCCAAGCTGGGCTGT |
| | GTGCACGAACCCCCCGTTCAGCCCGACCGCTGCGCCTTATCCGGTAACT |
| | ATCGTCTTGAGTCCAACCCGGTAAGACACGACTTATCGCCACTGGCAGC |
| | AGCCACTGGTAACAGGATTAGCAGAGCGAGGTATGTAGGCGGTGCTAC |
| | AGAGTTCTTGAAGTGGTGGCCTAACTACGGCTACACTAGAAGAACAGT |
| | ATTTGGTATCTGCGCTCTGCTGAAGCCAGTTACCTTCGGAAAAAGAGTT |
| | GGTAGCTCTTGATCCGGCAAACAAACCACCGCTGGTAGCGGTGGTTTTT |
| | TTGTTTGCAAGCAGCAGATTACGCGCAGAAAAAAAGGATCTCAAGAAG |
| | ATCCTTTGATCTTTTCTACGGGGTCTGACGCTCAGTGGAACGAAAACTC |
| | ACGTTAAGGGATTTTGGTCATGAGATTATCAAAAAGGATCTTCACCTAG |
| | ATCCTTTTAAATTAAAAATGAAGTTTTAAATCAATCTAAAGTATATATG |
| | AGTAAACTTGGTCTGACAGTTACCAATGCTTAATCAGTGAGGCACCTAT |
| | CTCAGCGATCTGTCTATTTCGTTCATCCATAGTTGCCTGACTCCCCGTCG |
| | TGTAGATAACTACGATACGGGAGGGCTTACCATCTGGCCCCAGTGCTGC |
| | AATGATACCGCGAGACCCACGCTCACCGGCTCCAGATTTATCAGCAATA |
| | AACCAGCCAGCCGGAAGGGCCGAGCGCAGAAGTGGTCCTGCAACTTTA |
| | TCCGCCTCCATCCAGTCTATTAATTGTTGCCGGGAAGCTAGAGTAAGTA |
| | GTTCGCCAGTTAATAGTTTGCGCAACGTTGTTGCCATTGCTACAGGCAT |
| | CGTGGTGTCACGCTCGTCGTTTGGTATGGCTTCATTCAGCTCCGGTTCCC |
| | AACGATCAAGGCGAGTTACATGATCCCCCATGTTGTGCAAAAAAGCGG |
| | TTAGCTCCTTCGGTCCTCCGATCGTTGTCAGAAGTAAGTTGGCCGCAGT |
| | GTTATCACTCATGGTTATGGCAGCACTGCATAATTCTCTTACTGTCATGC |
| | CATCCGTAAGATGCTTTTCTGTGACTGGTGAGTACTCAACCAAGTCATT |
| | CTGAGAATAGTGTATGCGGCGACCGAGTTGCTCTTGCCCGGCGTCAATA |
| | CGGGATAATACCGCGCCACATAGCAGAACTTTAAAAGTGCTCATCATTG |
| | GAAAACGTTCTTCGGGGCGAAAACTCTCAAGGATCTTACCGCTGTTGAG |
| | ATCCAGTTCGATGTAACCCACTCGTGCACCCAACTGATCTTCAGCATCT |
| | TTTACTTTCACCAGCGTTTCTGGGTGAGCAAAAACAGGAAGGCAAAAT |
| | GCCGCAAAAAAGGGAATAAGGGCGACACGGAAATGTTGAATACTCATA |
| | CTCTTCCTTTTTCAATATTATTGAAGCATTTATCAGGGTTATTGTCTCAT |
| | GAGCGGATACATATTTGAATGTATTTAGAAAAATAAACAAATAGGGGT |
| | TCCGCGCACATTTCCCCGAAAAGTGCCACCTAAATTGTAAGCGTTAATA |
| | TTTTGTTAAAATTCGCGTTAAATTTTTGTTAAATCAGCTCATTTTTTAAC |
| | CAATAGGCCGAAATCGGCAAAATCCCTTATAAATCAAAAGAATAGACC |
| | GAGATAGGGTTGAGTGTTGTTCCAGTTTGGAACAAGAGTCCACTATTAA |
| | AGAACGTGGACTCCAACGTCAAAGGGCGAAAAACCGTCTATCAGGGCG |
| | ATGGCCCACTACGTGAACCATCACCCTAATCAAGTTTTTTGGGGTCGAG |
| | GTGCCGTAAAGCACTAAATCGGAACCCTAAAGGGAGCCCCCGATTTAG |
| | AGCTTGACGGGGAAAGCCGGCGAACGTGGCGAGAAAGGAAGGGAAGA |
| | AAGCGAAAGGAGCGGGCGCTAGGGCGCTGGCAAGTGTAGCGGTCACGC |
| | TGCGCGTAACCACCACACCCGCCGCGCTTAATGCGCCGCTACAGGGCG |
| | CGTCCCATTCGCCATTCAG (SEQ ID NO: 3) |
| AAV2 | ACCTACACCGAACTGAGATACCTACAGCGTGAGCTATGAGAAAGCGCC |
| | ACGCTTCCCGAAGGGAGAAAGGCGGACAGGTATCCGGTAAGCGGCAGG |
| | GTCGGAACAGGAGAGCGCACGAGGGAGCTTCCAGGGGGAAACGCCTG |
| | GTATCTTTATAGTCCTGTCGGGTTTCGCCACCTCTGACTTGAGCGTCGAT |
| | TTTTGTGATGCTCGTCAGGGGGGCGGAGCCTATGGAAAAACGCCAGCA |
| | ACGCGGCCTTTTTACGGTTCCTGGCCTTTTGCTGGCCTTTTGCTCACATG |
| | TTCTTTCCTGCGTTATCCCCTGATTCTGTGGATAACCGTATTACCGCCTT |
| | TGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCT |
| | CCCCGCGCGTTGGCCGATTCATTAATGCAGCTGGCACGACAGGTTTCCC |
| | GACTGGAAAGCGGGCAGTGAGCGCAACGCAATTAATGTGAGTTAGCTC |
| | ACTCATTAGGCACCCCAGGCTTTACACTTTATGCTTCCGGCTCGTATGTT |
| | GTGTGGAATTGTGAGCGGATAACAATTTCACACAGGAAACAGCTATGA |
| | CCATGATTACGCCAAGCGCGCCGATATCGTTAACGCCCCGCGCCGGCCG |
| | CTCTAGAACTAGTGGATCCCCCGGAAGATCAGAAGTTCCTATTCCGAAG |
| | TTCCTATTCTCTAGAAAGTATAGGAACTTCTGATCTATTCGAGCTCGGT |
| | ACCCCTAGAGTCCTGTATTAGAGGTCACGTGAGTGTTTTGCGACATTTT |
| | GCGACACCATGTGGTCACGCTGGGTATTTAAGCCCGAGTGAGCACGCA |
| | GGGTCTCCATTTTGAAGCGGGAGGTTTGAACGCGCAGCCGCCATGCCG |
| | GGGTTTTACGAGATTGTGATTAAGGTCCCCAGCGACCTTGACGAGCATC |
| | TGCCCGGCATTTCTGACAGCTTTGTGAACTGGGTGGCCGAGAAGGAATG |
| | GGAGTTGCCGCCAGATTCTGACATGGATCTGAATCTGATTGAGCAGGCA |
| | CCCCTGACCGTGGCCGAGAAGCTGCAGCGCGACTTTCTGACGGAATGG |
| | CGCCGTGTGAGTAAGGCCCCGGAGGCCCTTTTCTTTGTGCAATTTGAGA |
| | AGGGAGAGAGCTACTTCCACATGCACGTGCTCGTGGAAACCACCGGGG |
| | TGAAATCCATGGTTTTGGGACGTTTCCTGAGTCAGATTCGCGAAAAACT |
| | GATTCAGAGAATTTACCGCGGGATCGAGCCGACTTTGCCAAACTGGTTC |
| | GCGGTCACAAAGACCAGAAATGGCGCCGGAGGCGGGAACAAGGTGGT |
| | GGATGAGTGCTACATCCCCAATTACTTGCTCCCCAAAACCCAGCCTGAG |
| | CTCCAGTGGGCGTGGACTAATATGGAACAGTATTTAAGCGCCTGTTTGA |
| | ATCTCACGGAGCGTAAACGGTTGGTGGCGCAGCATCTGACGCACGTGT |
| | CGCAGACGCAGGAGCAGAACAAAGAGAATCAGAATCCCAATTCTGATG |
| | CGCCGGTGATCAGATCAAAAACTTCAGCCAGGTACATGGAGCTGGTCG |
| | GGTGGCTCGTGGACAAGGGGATTACCTCGGAGAAGCAGTGGATCCAGG |
| | AGGACCAGGCCTCATACATCTCCTTCAATGCGGCCTCCAACTCGCGGTC |
| | CCAAATCAAGGCTGCCTTGGACAATGCGGGAAAGATTATGAGCCTGAC |
| | TAAAACCGCCCCCGACTACCTGGTGGGCCAGCAGCCCGTGGAGGACAT |
| | TTCCAGCAATCGGATTTATAAAATTTTGGAACTAAACGGGTACGATCCC |
| | CAATATGCGGCTTCCGTCTTTCTGGGATGGGCCACGAAAAAGTTCGGCA |
| | AGAGGAACACCATCTGGCTGTTTGGGCCTGCAACTACCGGGAAGACCA |
| | ACATCGCGGAGGCCATAGCCCACACTGTGCCCTTCTACGGGTGCGTAAA |
| | CTGGACCAATGAGAACTTTCCCTTCAACGACTGTGTCGACAAGATGGTG |
| | ATCTGGTGGGAGGAGGGGAAGATGACCGCCAAGGTCGTGGAGTCGGCC |
| | AAAGCCATTCTCGGAGGAAGCAAGGTGCGCGTGGACCAGAAATGCAAG |
| | TCCTCGGCCCAGATAGACCCGACTCCCGTGATCGTCACCTCCAACACCA |
| | ACATGTGCGCCGTGATTGACGGGAACTCAACGACCTTCGAACACCAGC |
| | AGCCGTTGCAAGACCGGATGTTCAAATTTGAACTCACCCGCCGTCTGGA |
| | TCATGACTTTGGGAAGGTCACCAAGCAGGAAGTCAAAGACTTTTTCCGG |
| | TGGGCAAAGGATCACGTGGTTGAGGTGGAGCATGAATTCTACGTCAAA |
| | AAGGGTGGAGCCAAGAAAAGACCCGCCCCCAGTGACGCAGATATAAGT |
| | GAGCCCAAACGGGTGCGCGAGTCAGTTGCGCAGCCATCGACGTCAGAC |
| | GCGGAAGCTTCGATCAACTACGCAGACAGGTACCAAAACAAATGTTCT |
| | CGTCACGTGGGCATGAATCTGATGCTGTTTCCCTGCAGACAATGCGAGA |
| | GAATGAATCAGAATTCAAATATCTGCTTCACTCACGGACAGAAAGACT |
| | GTTTAGAGTGCTTTCCCGTGTCAGAATCTCAACCCGTTTCTGTCGTCAAA |
| | AAGGCGTATCAGAAACTGTGCTACATTCATCATATCATGGGAAAGGTG |
| | CCAGACGCTTGCACTGCCTGCGATCTGGTCAATGTGGATTTGGATGACT |
| | GCATCTTTGAACAATAAATGATTTAAATCAGGTATGGCTGCCGATGGTT |
| | ATCTTCCAGATTGGCTCGAGGACACTCTCTCTGAAGGAATAAGACAGTG |
| | GTGGAAGCTCAAACCTGGCCCACCACCACCAAAGCCCGCAGAGCGGCA |
| | TAAGGACGACAGCAGGGGTCTTGTGCTTCCTGGGTACAAGTACCTCGG |
| | ACCCTTCAACGGACTCGACAAGGGAGAGCCGGTCAACGAGGCAGACGC |
| | CGCGGCCCTCGAGCACGACAAAGCCTACGACCGGCAGCTCGACAGCGG |
| | AGACAACCCGTACCTCAAGTACAACCACGCCGACGCGGAGTTTCAGGA |
| | GCGCCTTAAAGAAGATACGTCTTTTGGGGGCAACCTCGGACGAGCAGT |
| | CTTCCAGGCGAAAAAGAGGGTTCTTGAACCTCTGGGCCTGGTTGAGGA |
| | ACCTGTTAAGACGGCTCCGGGAAAAAAGAGGCCGGTAGAGCACTCTCC |
| | TGTGGAGCCAGACTCCTCCTCGGGAACCGGAAAGGCGGGCCAGCAGCC |
| | TGCAAGAAAAAGATTGAATTTTGGTCAGACTGGAGACGCAGACTCAGT |
| | ACCTGACCCCCAGCCTCTCGGACAGCCACCAGCAGCCCCCTCTGGTCTG |
| | GGAACTAATACGATGGCTACAGGCAGTGGCGCACCAATGGCAGACAAT |
| | AACGAGGGCGCCGACGGAGTGGGTAATTCCTCGGGAAATTGGCATTGC |
| | GATTCCACATGGATGGGCGACAGAGTCATCACCACCAGCACCCGAACC |
| | TGGGCCCTGCCCACCTACAACAACCACCTCTACAAACAAATTTCCAGCC |
| | AATCAGGAGCCTCGAACGACAATCACTACTTTGGCTACAGCACCCCTTG |
| | GGGGTATTTTGACTTCAACAGATTCCACTGCCACTTTTCACCACGTGAC |
| | TGGCAAAGACTCATCAACAACAACTGGGGATTCCGACCCAAGAGACTC |
| | AACTTCAAGCTCTTTAACATTCAAGTCAAAGAGGTCACGCAGAATGAC |
| | GGTACGACGACGATTGCCAATAACCTTACCAGCACGGTTCAGGTGTTTA |
| | CTGACTCGGAGTACCAGCTCCCGTACGTCCTCGGCTCGGCGCATCAAGG |
| | ATGCCTCCCGCCGTTCCCAGCAGACGTCTTCATGGTGCCACAGTATGGA |
| | TACCTCACCCTGAACAACGGGAGTCAGGCAGTAGGACGCTCTTCATTTT |
| | ACTGCCTGGAGTACTTTCCTTCTCAGATGCTGCGTACCGGAAACAACTT |
| | TACCTTCAGCTACACTTTTGAGGACGTTCCTTTCCACAGCAGCTACGCTC |
| | ACAGCCAGAGTCTGGACCGTCTCATGAATCCTCTCATCGACCAGTACCT |
| | GTATTACTTGAGCAGAACAAACACTCCAAGTGGAACCACCACGCAGTC |
| | AAGGCTTCAGTTTTCTCAGGCCGGAGCGAGTGACATTCGGGACCAGTCT |
| | AGGAACTGGCTTCCTGGACCCTGTTACCGCCAGCAGCGAGTATCAAAG |
| | ACATCTGCGGATAACAACAACAGTGAATACTCGTGGACTGGAGCTACC |
| | AAGTACCACCTCAATGGCAGAGACTCTCTGGTGAATCCGGGCCCGGCC |
| | ATGGCAAGCCACAAGGACGATGAAGAAAAGTTTTTTCCTCAGAGCGGG |
| | GTTCTCATCTTTGGGAAGCAAGGCTCAGAGAAAACAAATGTGGACATT |
| | GAAAAGGTCATGATTACAGACGAAGAGGAAATCAGGACAACCAATCCC |
| | GTGGCTACGGAGCAGTATGGTTCTGTATCTACCAACCTCCAGAGAGGCA |
| | ACAGACAAGCAGCTACCGCAGATGTCAACACACAAGGCGTTCTTCCAG |
| | GCATGGTCTGGCAGGACAGAGATGTGTACCTTCAGGGGCCCATCTGGG |
| | CAAAGATTCCACACACGGACGGACATTTTCACCCCTCTCCCCTCATGGG |
| | TGGATTCGGACTTAAACACCCTCCTCCACAGATTCTCATCAAGAACACC |
| | CCGGTACCTGCGAATCCTTCGACCACCTTCAGTGCGGCAAAGTTTGCTT |
| | CCTTCATCACACAGTACTCCACGGGACAGGTCAGCGTGGAGATCGAGT |
| | GGGAGCTGCAGAAGGAAAACAGCAAACGCTGGAATCCCGAAATTCAGT |
| | ACACTTCCAACTACAACAAGTCTGTTAATGTGGACTTTACTGTGGACAC |
| | TAATGGCGTGTATTCAGAGCCTCGCCCCATTGGCACCAGATACCTGACT |
| | CGTAATCTGTAATTGCTTGTTAATCAATAAACCGTTTAATTCGTTTCAGT |
| | TGAACTTTGGTCTCTGCGTATTTCTTTCTTATCTAGTTTCCATGGCTACG |
| | TAGATAAGTAGCATGGCGGGTTAATCATTAACTACAGCCCGGGCGTTTA |
| | AACAGCGGGCGGAGGGGTGGAGTCGTGACGTGAATTACGTCATAGGGT |
| | TAGGGAGAGTCCTGTATTAGAGGTCACGTGAGTGTTTTGCGACATTTTG |
| | CGACACCATGTGGTCACGCTGGGTATTTAAGCCCGAGTGAGCACGCAG |
| | GGTCTCCATTTTGAGCGGGAGGTTTGAACGAGCGCTGGCGCGCTCACTG |
| | GCCGTCGTTTTACAACGTCGTGACTGGGAAAACCCTGGCGTTACCCAAC |
| | TTAATCGCCTTGCAGCACATCCCCCTTTCGCCAGCTGGCGTAATAGCGA |
| | AGAGGCCCGCACCGATCGCCCTTCCCAACAGTTGCGCAGCCTGAATGG |
| | CGAATGGAAATTGTAAGCGTTAATATTTTGTTAAAATTCGCGTTAAATT |
| | TTTGTTAAATCAGCTCATTTTTTTAACCAATAGGCCGAAATCGGCAAAA |
| | TCCCTTATAAATCAAAAGAATAGACCGAGATAGGGTTGAGTGTTGTTCC |
| | AGTTTGGAACAAGAGTCCACTATTAAGAACGTGGACTCCAACGTCAAA |
| | GGGCGAAAAACCGTCTATCAGGGCGATGGCCCACTACGTGAACCATCA |
| | CCCTAATCAAGTTTTTTGGGGTCGAGGTGCCGTAAAGCACTAAATCGGA |
| | ACCCTAAAGGGAGCCCCCGATTTAGAGCTTGACGGGGAAAGCCGGCGA |
| | ACGTGGCGAGAAAGGAAGGGAAGAAAGCGAAAGGAGCGGGCGCTAGG |
| | GCGCTGGCAAGTGTAGCGGTCACGCTGCGCGTAACCACCACACCCGCC |
| | GCGCTTAATGCGCCGCTACAGGGCGCGTCAGGTGGCACTTTTCGGGGA |
| | AATGTGCGCGGAACCCCTATTTGTTTATTTTTCTAAATACATTCAAATAT |
| | GTATCCGCTCATGAGACAATAACCCTGATAAATGCTTCAATAATATTGA |
| | AAAAGGAAGAGTATGAGTATTCAACATTTCCGTGTCGCCCTTATTCCCT |
| | TTTTTGCGGCATTTTGCCTTCCTGTTTTTGCTCACCCAGAAACGCTGGTG |
| | AAAGTAAAAGATGCTGAAGATCAGTTGGGTGCACGAGTGGGTTACATC |
| | GAACTGGATCTCAACAGCGGTAAGATCCTTGAGAGTTTTCGCCCCGAAG |
| | AACGTTTTCCAATGATGAGCACTTTTAAAGTTCTGCTATGTGGCGCGGT |
| | ATTATCCCGTATTGACGCCGGGCAAGAGCAACTCGGTCGCCGCATACAC |
| | TATTCTCAGAATGACTTGGTTGAGTACTCACCAGTCACAGAAAAGCATC |
| | TTACGGATGGCATGACAGTAAGAGAATTATGCAGTGCTGCCATAACCA |
| | TGAGTGATAACACTGCGGCCAACTTACTTCTGACAACGATCGGAGGAC |
| | CGAAGGAGCTAACCGCTTTTTTGCACAACATGGGGGATCATGTAACTCG |
| | CCTTGATCGTTGGGAACCGGAGCTGAATGAAGCCATACCAAACGACGA |
| | GCGTGACACCACGATGCCTGTAGCAATGGCAACAACGTTGCGCAAACT |
| | ATTAACTGGCGAACTACTTACTCTAGCTTCCCGGCAACAATTAATAGAC |
| | TGGATGGAGGCGGATAAAGTTGCAGGACCACTTCTGCGCTCGGCCCTTC |
| | CGGCTGGCTGGTTTATTGCTGATAAATCTGGAGCCGGTGAGCGTGGGTC |
| | TCGCGGTATCATTGCAGCACTGGGGCCAGATGGTAAGCCCTCCCGTATC |
| | GTAGTTATCTACACGACGGGGAGTCAGGCAACTATGGATGAACGAAAT |
| | AGACAGATCGCTGAGATAGGTGCCTCACTGATTAAGCATTGGTAACTGT |
| | CAGACCAAGTTTACTCATATATACTTTAGATTGATTTAAAACTTCATTTT |
| | TAATTTAAAAGGATCTAGGTGAAGATCCTTTTTGATAATCTCATGACCA |
| | AAATCCCTTAACGTGAGTTTTCGTTCCACTGAGCGTCAGACCCCGTAGA |
| | AAAGATCAAAGGATCTTCTTGAGATCCTTTTTTTCTGCGCGTAATCTGCT |
| | GCTTGCAAACAAAAAAACCACCGCTACCAGCGGTGGTTTGTTTGCCGG |
| | ATCAAGAGCTACCAACTCTTTTTCCGAAGGTAACTGGCTTCAGCAGAGC |
| | GCAGATACCAAATACTGTTCTTCTAGTGTAGCCGTAGTTAGGCCACCAC |
| | TTCAAGAACTCTGTAGCACCGCCTACATACCTCGCTCTGCTAATCCTGTT |
| | ACCAGTGGCTGCTGCCAGTGGCGATAAGTCGTGTCTTACCGGGTTGGAC |
| | TCAAGACGATAGTTACCGGATAAGGCGCAGCGGTCGGGCTGAACGGGG |
| | GGTTCGTGCACACAGCCCAGCTTGGAGCGAACG (SEQ ID NO: 4) |
| AAV8 | ATGCCGGGGTTTTACGAGATTGTGATTAAGGTCCCCAGCGACCTTGACG |
| | AGCATCTGCCCGGCATTTCTGACAGCTTTGTGAACTGGGTGGCCGAGAA |
| | GGAATGGGAGTTGCCGCCAGATTCTGACATGGATCTGAATCTGATTGAG |
| | CAGGCACCCCTGACCGTGGCCGAGAAGCTGCAGCGCGACTTTCTGACG |
| | GAATGGCGCCGTGTGAGTAAGGCCCCGGAGGCTCTTTTCTTTGTGCAAT |
| | TTGAGAAGGGAGAGAGCTACTTCCACATGCACGTGCTCGTGGAAACCA |
| | CCGGGGTGAAATCCATGGTTTTGGGACGTTTCCTGAGTCAGATTCGCGA |
| | AAAACTGATTCAGAGAATTTACCGCGGGATCGAGCCGACTTTGCCAAA |
| | CTGGTTCGCGGTCACAAAGACCAGAAATGGCGCCGGAGGCGGGAACAA |
| | GGTGGTGGATGAGTGCTACATCCCCAATTACTTGCTCCCCAAAACCCAG |
| | CCTGAGCTCCAGTGGGCGTGGACTAATATGGAACAGTATTTAAGCGCCT |
| | GTTTGAATCTCACGGAGCGTAAACGGTTGGTGGCGCAGCATCTGACGC |
| | ACGTGTCGCAGACGCAGGAGCAGAACAAAGAGAATCAGAATCCCAATT |
| | CTGATGCGCCGGTGATCAGATCAAAAACTTCAGCCAGGTACATGGAGC |
| | TGGTCGGGTGGCTCGTGGACAAGGGGATTACCTCGGAGAAGCAGTGGA |
| | TCCAGGAGGACCAGGCCTCATACATCTCCTTCAATGCGGCCTCCAACTC |
| | GCGGTCCCAAATCAAGGCTGCCTTGGACAATGCGGGAAAGATTATGAG |
| | CCTGACTAAAACCGCCCCCGACTACCTGGTGGGCCAGCAGCCCGTGGA |
| | GGACATTTCCAGCAATCGGATTTATAAAATTTTGGAACTAAACGGGTAC |
| | GATCCCCAATATGCGGCTTCCGTCTTTCTGGGATGGGCCACGAAAAAGT |
| | TCGGCAAGAGGAACACCATCTGGCTGTTTGGGCCTGCAACTACCGGGA |
| | AGACCAACATCGCGGAGGCCATAGCCCACACTGTGCCCTTCTACGGGT |
| | GCGTAAACTGGACCAATGAGAACTTTCCCTTCAACGACTGTGTCGACAA |
| | GATGGTGATCTGGTGGGAGGAGGGGAAGATGACCGCCAAGGTCGTGGA |
| | GTCGGCCAAAGCCATTCTCGGAGGAAGCAAGGTGCGCGTGGACCAGAA |
| | ATGCAAGTCCTCGGCCCAGATAGACCCGACTCCCGTGATCGTCACCTCC |
| | AACACCAACATGTGCGCCGTGATTGACGGGAACTCAACGACCTTCGAA |
| | CACCAGCAGCCGTTGCAAGACCGGATGTTCAAATTTGAACTCACCCGCC |
| | GTCTGGATCATGACTTTGGGAAGGTCACCAAGCAGGAAGTCAAAGACT |
| | TTTTCCGGTGGGCAAAGGATCACGTGGTTGAGGTGGAGCATGAATTCTA |
| | CGTCAAAAAGGGTGGAGCCAAGAAAAGACCCGCCCCCAGTGACGCAG |
| | ATATAAGTGAGCCCAAACGGGTGCGCGAGTCAGTTGCGCAGCCATCGA |
| | CGTCAGACGCGGAAGCTTCGATCAACTACGCAGACAGGTACCAAAACA |
| | AATGTTCTCGTCACGTGGGCATGAATCTGATGCTGTTTCCCTGCAGACA |
| | ATGCGAGAGAATGAATCAGAATTCAAATATCTGCTTCACTCACGGACA |
| | GAAAGACTGTTTAGAGTGCTTTCCCGTGTCAGAATCTCAACCCGTTTCT |
| | GTCGTCAAAAAGGCGTATCAGAAACTGTGCTACATTCATCATATCATGG |
| | GAAAGGTGCCAGACGCTTGCACTGCCTGCGATCTGGTCAATGTGGATTT |
| | GGATGACTGCATCTTTGAACAATAAATGATTTAAATCAGGTATGGCTGC |
| | CGATGGTTATCTTCCAGATTGGCTCGAGGACAACCTCTCTGAGGGCATT |
| | CGCGAGTGGTGGGCGCTGAAACCTGGAGCCCCGAAGCCCAAAGCCAAC |
| | CAGCAAAAGCAGGACGACGGCCGGGGTCTGGTGCTTCCTGGCTACAAG |
| | TACCTCGGACCCTTCAACGGACTCGACAAGGGGGAGCCCGTCAACGCG |
| | GCGGACGCAGCGGCCCTCGAGCACGACAAGGCCTACGACCAGCAGCTG |
| | CAGGCGGGTGACAATCCGTACCTGCGGTATAACCACGCCGACGCCGAG |
| | TTTCAGGAGCGTCTGCAAGAAGATACGTCTTTTGGGGGCAACCTCGGGC |
| | GAGCAGTCTTCCAGGCCAAGAAGCGGGTTCTCGAACCTCTCGGTCTGGT |
| | TGAGGAAGGCGCTAAGACGGCTCCTGGAAAGAAGAGACCGGTAGAGC |
| | CATCACCCCAGCGTTCTCCAGACTCCTCTACGGGCATCGGCAAGAAAGG |
| | CCAACAGCCCGCCAGAAAAAGACTCAATTTTGGTCAGACTGGCGACTC |
| | AGAGTCAGTTCCAGACCCTCAACCTCTCGGAGAACCTCCAGCAGCGCCC |
| | TCTGGTGTGGGACCTAATACAATGGCTGCAGGCGGTGGCGCACCAATG |
| | GCAGACAATAACGAAGGCGCCGACGGAGTGGGTAGTTCCTCGGGAAAT |
| | TGGCATTGCGATTCCACATGGCTGGGCGACAGAGTCATCACCACCAGC |
| | ACCCGAACCTGGGCCCTGCCCACCTACAACAACCACCTCTACAAGCAA |
| | ATCTCCAACGGGACATCGGGAGGAGCCACCAACGACAACACCTACTTC |
| | GGCTACAGCACCCCCTGGGGGTATTTTGACTTTAACAGATTCCACTGCC |
| | ACTTTTCACCACGTGACTGGCAGCGACTCATCAACAACAACTGGGGATT |
| | CCGGCCCAAGAGACTCAGCTTCAAGCTCTTCAACATCCAGGTCAAGGA |
| | GGTCACGCAGAATGAAGGCACCAAGACCATCGCCAATAACCTCACCAG |
| | CACCATCCAGGTGTTTACGGACTCGGAGTACCAGCTGCCGTACGTTCTC |
| | GGCTCTGCCCACCAGGGCTGCCTGCCTCCGTTCCCGGCGGACGTGTTCA |
| | TGATTCCCCAGTACGGCTACCTAACACTCAACAACGGTAGTCAGGCCGT |
| | GGGACGCTCCTCCTTCTACTGCCTGGAATACTTTCCTTCGCAGATGCTG |
| | AGAACCGGCAACAACTTCCAGTTTACTTACACCTTCGAGGACGTGCCTT |
| | TCCACAGCAGCTACGCCCACAGCCAGAGCTTGGACCGGCTGATGAATC |
| | CTCTGATTGACCAGTACCTGTACTACTTGTCTCGGACTCAAACAACAGG |
| | AGGCACGGCAAATACGCAGACTCTGGGCTTCAGCCAAGGTGGGCCTAA |
| | TACAATGGCCAATCAGGCAAAGAACTGGCTGCCAGGACCCTGTTACCG |
| | CCAACAACGCGTCTCAACGACAACCGGGCAAAACAACAATAGCAACTT |
| | TGCCTGGACTGCTGGGACCAAATACCATCTGAATGGAAGAAATTCATTG |
| | GCTAATCCTGGCATCGCTATGGCAACACACAAAGACGACGAGGAGCGT |
| | TTTTTTCCCAGTAACGGGATCCTGATTTTTGGCAAACAAAATGCTGCCA |
| | GAGACAATGCGGATTACAGCGATGTCATGCTCACCAGCGAGGAAGAAA |
| | TCAAAACCACTAACCCTGTGGCTACAGAGGAATACGGTATCGTGGCAG |
| | ATAACTTGCAGCAGCAAAACACGGCTCCTCAAATTGGAACTGTCAACA |
| | GCCAGGGGGCCTTACCCGGTATGGTCTGGCAGAACCGGGACGTGTACC |
| | TGCAGGGTCCCATCTGGGCCAAGATTCCTCACACGGACGGCAACTTCCA |
| | CCCGTCTCCGCTGATGGGCGGCTTTGGCCTGAAACATCCTCCGCCTCAG |
| | ATCCTGATCAAGAACACGCCTGTACCTGCGGATCCTCCGACCACCTTCA |
| | ACCAGTCAAAGCTGAACTCTTTCATCACGCAATACAGCACCGGACAGG |
| | TCAGCGTGGAAATTGAATGGGAGCTGCAGAAGGAAAACAGCAAGCGCT |
| | GGAACCCCGAGATCCAGTACACCTCCAACTACTACAAATCTACAAGTGT |
| | GGACTTTGCTGTTAATACAGAAGGCGTGTACTCTGAACCCCGCCCCATT |
| | GGCACCCGTTACCTCACCCGTAATCTGTAATTGCCTGTTAATCAATAAA |
| | CCGGTTGATTCGTTTCAGTTGAACTTTGGTCTCTGCGAAGGGCGAATTC |
| | GTTTAAACCTGCAGGACTAGAGGTCCTGTATTAGAGGTCACGTGAGTGT |
| | TTTGCGACATTTTGCGACACCATGTGGTCACGCTGGGTATTTAAGCCCG |
| | AGTGAGCACGCAGGGTCTCCATTTTGAAGCGGGAGGTTTGAACGCGCA |
| | GCCGCCAAGCCGAATTCTGCAGATATCCATCACACTGGCGGCCGCTCGA |
| | CTAGAGCGGCCGCCACCGCGGTGGAGCTCCAGCTTTTGTTCCCTTTAGT |
| | GAGGGTTAATTGCGCGCTTGGCGTAATCATGGTCATAGCTGTTTCCTGT |
| | GTGAAATTGTTATCCGCTCACAATTCCACACAACATACGAGCCGGAAGC |
| | ATAAAGTGTAAAGCCTGGGGTGCCTAATGAGTGAGCTAACTCACATTA |
| | ATTGCGTTGCGCTCACTGCCCGCTTTCCAGTCGGGAAACCTGTCGTGCC |
| | AGCTGCATTAATGAATCGGCCAACGCGCGGGGAGAGGCGGTTTGCGTA |
| | TTGGGCGCTCTTCCGCTTCCTCGCTCACTGACTCGCTGCGCTCGGTCGTT |
| | CGGCTGCGGCGAGCGGTATCAGCTCACTCAAAGGCGGTAATACGGTTA |
| | TCCACAGAATCAGGGGATAACGCAGGAAAGAACATGTGAGCAAAAGG |
| | CCAGCAAAAGGCCAGGAACCGTAAAAAGGCCGCGTTGCTGGCGTTTTT |
| | CCATAGGCTCCGCCCCCCTGACGAGCATCACAAAAATCGACGCTCAAG |
| | TCAGAGGTGGCGAAACCCGACAGGACTATAAAGATACCAGGCGTTTCC |
| | CCCTGGAAGCTCCCTCGTGCGCTCTCCTGTTCCGACCCTGCCGCTTACCG |
| | GATACCTGTCCGCCTTTCTCCCTTCGGGAAGCGTGGCGCTTTCTCATAGC |
| | TCACGCTGTAGGTATCTCAGTTCGGTGTAGGTCGTTCGCTCCAAGCTGG |
| | GCTGTGTGCACGAACCCCCCGTTCAGCCCGACCGCTGCGCCTTATCCGG |
| | TAACTATCGTCTTGAGTCCAACCCGGTAAGACACGACTTATCGCCACTG |
| | GCAGCAGCCACTGGTAACAGGATTAGCAGAGCGAGGTATGTAGGCGGT |
| | GCTACAGAGTTCTTGAAGTGGTGGCCTAACTACGGCTACACTAGAAGA |
| | ACAGTATTTGGTATCTGCGCTCTGCTGAAGCCAGTTACCTTCGGAAAAA |
| | GAGTTGGTAGCTCTTGATCCGGCAAACAAACCACCGCTGGTAGCGGTG |
| | GTTTTTTTGTTTGCAAGCAGCAGATTACGCGCAGAAAAAAAGGATCTCA |
| | AGAAGATCCTTTGATCTTTTCTACGGGGTCTGACGCTCAGTGGAACGAA |
| | AACTCACGTTAAGGGATTTTGGTCATGAGATTATCAAAAAGGATCTTCA |
| | CCTAGATCCTTTTAAATTAAAAATGAAGTTTTAAATCAATCTAAAGTAT |
| | ATATGAGTAAACTTGGTCTGACAGTTACCAATGCTTAATCAGTGAGGCA |
| | CCTATCTCAGCGATCTGTCTATTTCGTTCATCCATAGTTGCCTGACTCCC |
| | CGTCGTGTAGATAACTACGATACGGGAGGGCTTACCATCTGGCCCCAGT |
| | GCTGCAATGATACCGCGAGACCCACGCTCACCGGCTCCAGATTTATCAG |
| | CAATAAACCAGCCAGCCGGAAGGGCCGAGCGCAGAAGTGGTCCTGCAA |
| | CTTTATCCGCCTCCATCCAGTCTATTAATTGTTGCCGGGAAGCTAGAGT |
| | AAGTAGTTCGCCAGTTAATAGTTTGCGCAACGTTGTTGCCATTGCTACA |
| | GGCATCGTGGTGTCACGCTCGTCGTTTGGTATGGCTTCATTCAGCTCCG |
| | GTTCCCAACGATCAAGGCGAGTTACATGATCCCCCATGTTGTGCAAAAA |
| | AGCGGTTAGCTCCTTCGGTCCTCCGATCGTTGTCAGAAGTAAGTTGGCC |
| | GCAGTGTTATCACTCATGGTTATGGCAGCACTGCATAATTCTCTTACTGT |
| | CATGCCATCCGTAAGATGCTTTTCTGTGACTGGTGAGTACTCAACCAAG |
| | TCATTCTGAGAATAGTGTATGCGGCGACCGAGTTGCTCTTGCCCGGCGT |
| | CAATACGGGATAATACCGCGCCACATAGCAGAACTTTAAAAGTGCTCA |
| | TCATTGGAAAACGTTCTTCGGGGCGAAAACTCTCAAGGATCTTACCGCT |
| | GTTGAGATCCAGTTCGATGTAACCCACTCGTGCACCCAACTGATCTTCA |
| | GCATCTTTTACTTTCACCAGCGTTTCTGGGTGAGCAAAAACAGGAAGGC |
| | AAAATGCCGCAAAAAAGGGAATAAGGGCGACACGGAAATGTTGAATA |
| | CTCATACTCTTCCTTTTTCAATATTATTGAAGCATTTATCAGGGTTATTG |
| | TCTCATGAGCGGATACATATTTGAATGTATTTAGAAAAATAAACAAATA |
| | GGGGTTCCGCGCACATTTCCCCGAAAAGTGCCACCTAAATTGTAAGCGT |
| | TAATATTTTGTTAAAATTCGCGTTAAATTTTTGTTAAATCAGCTCATTTT |
| | TTAACCAATAGGCCGAAATCGGCAAAATCCCTTATAAATCAAAAGAAT |
| | AGACCGAGATAGGGTTGAGTGTTGTTCCAGTTTGGAACAAGAGTCCACT |
| | ATTAAAGAACGTGGACTCCAACGTCAAAGGGCGAAAAACCGTCTATCA |
| | GGGCGATGGCCCACTACGTGAACCATCACCCTAATCAAGTTTTTTGGGG |
| | TCGAGGTGCCGTAAAGCACTAAATCGGAACCCTAAAGGGAGCCCCCGA |
| | TTTAGAGCTTGACGGGGAAAGCCGGCGAACGTGGCGAGAAAGGAAGG |
| | GAAGAAAGCGAAAGGAGCGGGCGCTAGGGCGCTGGCAAGTGTAGCGG |
| | TCACGCTGCGCGTAACCACCACACCCGCCGCGCTTAATGCGCCGCTACA |
| | GGGCGCGTCCCATTCGCCATTCAGGCTGCGCAACTGTTGGGAAGGGCG |
| | ATCGGTGCGGGCCTCTTCGCTATTACGCCAGCTGGCGAAAGGGGGATGT |
| | GCTGCAAGGCGATTAAGTTGGGTAACGCCAGGGTTTTCCCAGTCACGAC |
| | GTTGTAAAACGACGGCCAGTGAGCGCGCGTAATACGACTCACTATAGG |
| | GCGAATTGGGTACCGGGCCCCCCCTCGATCGAGGTCGACGGTATCGGG |
| | GGAGCTCGCAGGGTCTCCATTTTGAAGCGGGAGGTTTGAACGCGCAGC |
| | CGCC (SEQ ID NO: 5) |
| AAV9 | TCGCCATTCAGGCTGCGCAACTGTTGGGAAGGGCGATCGGTGCGGGCC |
| | TCTTCGCTATTACGCCAGCTGGCGAAAGGGGGATGTGCTGCAAGGCGA |
| | TTAAGTTGGGTAACGCCAGGGTTTTCCCAGTCACGACGTTGTAAAACGA |
| | CGGCCAGTGAGCGCGCGTAATACGACTCACTATAGGGCGAATTGGGTA |
| | CCGGGCCCCCCCTCGATCGAGGTCGACGGTATCGGGGGAGCTCGCAGG |
| | GTCTCCATTTTGAAGCGGGAGGTTTGAACGCGCAGCCGCCATGCCGGG |
| | GTTTTACGAGATTGTGATTAAGGTCCCCAGCGACCTTGACGAGCATCTG |
| | CCCGGCATTTCTGACAGCTTTGTGAACTGGGTGGCCGAGAAGGAATGG |
| | GAGTTGCCGCCAGATTCTGACATGGATCTGAATCTGATTGAGCAGGCAC |
| | CCCTGACCGTGGCCGAGAAGCTGCAGCGCGACTTTCTGACGGAATGGC |
| | GCCGTGTGAGTAAGGCCCCGGAGGCTCTTTTCTTTGTGCAATTTGAGAA |
| | GGGAGAGAGCTACTTCCACATGCACGTGCTCGTGGAAACCACCGGGGT |
| | GAAATCCATGGTTTTGGGACGTTTCCTGAGTCAGATTCGCGAAAAACTG |
| | ATTCAGAGAATTTACCGCGGGATCGAGCCGACTTTGCCAAACTGGTTCG |
| | CGGTCACAAAGACCAGAAATGGCGCCGGAGGCGGGAACAAGGTGGTG |
| | GATGAGTGCTACATCCCCAATTACTTGCTCCCCAAAACCCAGCCTGAGC |
| | TCCAGTGGGCGTGGACTAATATGGAACAGTATTTAAGCGCCTGTTTGAA |
| | TCTCACGGAGCGTAAACGGTTGGTGGCGCAGCATCTGACGCACGTGTC |
| | GCAGACGCAGGAGCAGAACAAAGAGAATCAGAATCCCAATTCTGATGC |
| | GCCGGTGATCAGATCAAAAACTTCAGCCAGGTACATGGAGCTGGTCGG |
| | GTGGCTCGTGGACAAGGGGATTACCTCGGAGAAGCAGTGGATCCAGGA |
| | GGACCAGGCCTCATACATCTCCTTCAATGCGGCCTCCAACTCGCGGTCC |
| | CAAATCAAGGCTGCCTTGGACAATGCGGGAAAGATTATGAGCCTGACT |
| | AAAACCGCCCCCGACTACCTGGTGGGCCAGCAGCCCGTGGAGGACATT |
| | TCCAGCAATCGGATTTATAAAATTTTGGAACTAAACGGGTACGATCCCC |
| | AATATGCGGCTTCCGTCTTTCTGGGATGGGCCACGAAAAAGTTCGGCAA |
| | GAGGAACACCATCTGGCTGTTTGGGCCTGCAACTACCGGGAAGACCAA |
| | CATCGCGGAGGCCATAGCCCACACTGTGCCCTTCTACGGGTGCGTAAAC |
| | TGGACCAATGAGAACTTTCCCTTCAACGACTGTGTCGACAAGATGGTGA |
| | TCTGGTGGGAGGAGGGGAAGATGACCGCCAAGGTCGTGGAGTCGGCCA |
| | AAGCCATTCTCGGAGGAAGCAAGGTGCGCGTGGACCAGAAATGCAAGT |
| | CCTCGGCCCAGATAGACCCGACTCCCGTGATCGTCACCTCCAACACCAA |
| | CATGTGCGCCGTGATTGACGGGAACTCAACGACCTTCGAACACCAGCA |
| | GCCGTTGCAAGACCGGATGTTCAAATTTGAACTCACCCGCCGTCTGGAT |
| | CATGACTTTGGGAAGGTCACCAAGCAGGAAGTCAAAGACTTTTTCCGGT |
| | GGGCAAAGGATCACGTGGTTGAGGTGGAGCATGAATTCTACGTCAAAA |
| | AGGGTGGAGCCAAGAAAAGACCCGCCCCCAGTGACGCAGATATAAGTG |
| | AGCCCAAACGGGTGCGCGAGTCAGTTGCGCAGCCATCGACGTCAGACG |
| | CGGAAGCTTCGATCAACTACGCAGACAGGTACCAAAACAAATGTTCTC |
| | GTCACGTGGGCATGAATCTGATGCTGTTTCCCTGCAGACAATGCGAGAG |
| | AATGAATCAGAATTCAAATATCTGCTTCACTCACGGACAGAAAGACTGT |
| | TTAGAGTGCTTTCCCGTGTCAGAATCTCAACCCGTTTCTGTCGTCAAAA |
| | AGGCGTATCAGAAACTGTGCTACATTCATCATATCATGGGAAAGGTGCC |
| | AGACGCTTGCACTGCCTGCGATCTGGTCAATGTGGATTTGGATGACTGC |
| | ATCTTTGAACAATAAATGATTTAAATCAGGTATGGCTGCCGATGGTTAT |
| | CTTCCAGATTGGCTCGAGGACAACCTTAGTGAAGGAATTCGCGAGTGGT |
| | GGGCTTTGAAACCTGGAGCCCCTCAACCCAAGGCAAATCAACAACATC |
| | AAGACAACGCTCGAGGTCTTGTGCTTCCGGGTTACAAATACCTTGGACC |
| | CGGCAACGGACTCGACAAGGGGGAGCCGGTCAACGCAGCAGACGCGG |
| | CGGCCCTCGAGCACGACAAGGCCTACGACCAGCAGCTCAAGGCCGGAG |
| | ACAACCCGTACCTCAAGTACAACCACGCCGACGCCGAGTTCCAGGAGC |
| | GGCTCAAAGAAGATACGTCTTTTGGGGGCAACCTCGGGCGAGCAGTCT |
| | TCCAGGCCAAAAAGAGGCTTCTTGAACCTCTTGGTCTGGTTGAGGAAGC |
| | GGCTAAGACGGCTCCTGGAAAGAAGAGGCCTGTAGAGCAGTCTCCTCA |
| | GGAACCGGACTCCTCCGCGGGTATTGGCAAATCGGGTGCACAGCCCGC |
| | TAAAAAGAGACTCAATTTCGGTCAGACTGGCGACACAGAGTCAGTCCC |
| | AGACCCTCAACCAATCGGAGAACCTCCCGCAGCCCCCTCAGGTGTGGG |
| | ATCTCTTACAATGGCTTCAGGTGGTGGCGCACCAGTGGCAGACAATAAC |
| | GAAGGTGCCGATGGAGTGGGTAGTTCCTCGGGAAATTGGCATTGCGAT |
| | TCCCAATGGCTGGGGGACAGAGTCATCACCACCAGCACCCGAACCTGG |
| | GCCCTGCCCACCTACAACAATCACCTCTACAAGCAAATCTCCAACAGCA |
| | CATCTGGAGGATCTTCAAATGACAACGCCTACTTCGGCTACAGCACCCC |
| | CTGGGGGTATTTTGACTTCAACAGATTCCACTGCCACTTCTCACCACGT |
| | GACTGGCAGCGACTCATCAACAACAACTGGGGATTCCGGCCTAAGCGA |
| | CTCAACTTCAAGCTCTTCAACATTCAGGTCAAAGAGGTTACGGACAACA |
| | ATGGAGTCAAGACCATCGCCAATAACCTTACCAGCACGGTCCAGGTCTT |
| | CACGGACTCAGACTATCAGCTCCCGTACGTGCTCGGGTCGGCTCACGAG |
| | GGCTGCCTCCCGCCGTTCCCAGCGGACGTTTTCATGATTCCTCAGTACG |
| | GGTATCTGACGCTTAATGATGGAAGCCAGGCCGTGGGTCGTTCGTCCTT |
| | TTACTGCCTGGAATATTTCCCGTCGCAAATGCTAAGAACGGGTAACAAC |
| | TTCCAGTTCAGCTACGAGTTTGAGAACGTACCTTTCCATAGCAGCTACG |
| | CTCACAGCCAAAGCCTGGACCGACTAATGAATCCACTCATCGACCAAT |
| | ACTTGTACTATCTCTCAAAGACTATTAACGGTTCTGGACAGAATCAACA |
| | AACGCTAAAATTCAGTGTGGCCGGACCCAGCAACATGGCTGTCCAGGG |
| | AAGAAACTACATACCTGGACCCAGCTACCGACAACAACGTGTCTCAAC |
| | CACTGTGACTCAAAACAACAACAGCGAATTTGCTTGGCCTGGAGCTTCT |
| | TCTTGGGCTCTCAATGGACGTAATAGCTTGATGAATCCTGGACCTGCTA |
| | TGGCCAGCCACAAAGAAGGAGAGGACCGTTTCTTTCCTTTGTCTGGATC |
| | TTTAATTTTTGGCAAACAAGGAACTGGAAGAGACAACGTGGATGCGGA |
| | CAAAGTCATGATAACCAACGAAGAAGAAATTAAAACTACTAACCCGGT |
| | AGCAACGGAGTCCTATGGACAAGTGGCCACAAACCACCAGAGTGCCCA |
| | AGCACAGGCGCAGACCGGCTGGGTTCAAAACCAAGGAATACTTCCGGG |
| | TATGGTTTGGCAGGACAGAGATGTGTACCTGCAAGGACCCATTTGGGCC |
| | AAAATTCCTCACACGGACGGCAACTTTCACCCTTCTCCGCTGATGGGAG |
| | GGTTTGGAATGAAGCACCCGCCTCCTCAGATCCTCATCAAAAACACACC |
| | TGTACCTGCGGATCCTCCAACGGCCTTCAACAAGGACAAGCTGAACTCT |
| | TTCATCACCCAGTATTCTACTGGCCAAGTCAGCGTGGAGATCGAGTGGG |
| | AGCTGCAGAAGGAAAACAGCAAGCGCTGGAACCCGGAGATCCAGTAC |
| | ACTTCCAACTATTACAAGTCTAATAATGTTGAATTTGCTGTTAATACTG |
| | AAGGTGTATATAGTGAACCCCGCCCCATTGGCACCAGATACCTGACTCG |
| | TAATCTGTAATTGCTTGTTAATCAATAAACCGTTTAATTCGTTTCAGTTG |
| | AACTTTGGTCTCTGCGAAGGGCGAATTCGTTTAAACCTGCAGGACTAGA |
| | GGTCCTGTATTAGAGGTCACGTGAGTGTTTTGCGACATTTTGCGACACC |
| | ATGTGGTCACGCTGGGTATTTAAGCCCGAGTGAGCACGCAGGGTCTCCA |
| | TTTTGAAGCGGGAGGTTTGAACGCGCAGCCGCCAAGCCGAATTCTGCA |
| | GATATCCATCACACTGGCGGCCGCTCGACTAGAGCGGCCGCCACCGCG |
| | GTGGAGCTCCAGCTTTTGTTCCCTTTAGTGAGGGTTAATTGCGCGCTTG |
| | GCGTAATCATGGTCATAGCTGTTTCCTGTGTGAAATTGTTATCCGCTCAC |
| | AATTCCACACAACATACGAGCCGGAAGCATAAAGTGTAAAGCCTGGGG |
| | TGCCTAATGAGTGAGCTAACTCACATTAATTGCGTTGCGCTCACTGCCC |
| | GCTTTCCAGTCGGGAAACCTGTCGTGCCAGCTGCATTAATGAATCGGCC |
| | AACGCGCGGGGAGAGGCGGTTTGCGTATTGGGCGCTCTTCCGCTTCCTC |
| | GCTCACTGACTCGCTGCGCTCGGTCGTTCGGCTGCGGCGAGCGGTATCA |
| | GCTCACTCAAAGGCGGTAATACGGTTATCCACAGAATCAGGGGATAAC |
| | GCAGGAAAGAACATGTGAGCAAAAGGCCAGCAAAAGGCCAGGAACCG |
| | TAAAAAGGCCGCGTTGCTGGCGTTTTTCCATAGGCTCCGCCCCCCTGAC |
| | GAGCATCACAAAAATCGACGCTCAAGTCAGAGGTGGCGAAACCCGACA |
| | GGACTATAAAGATACCAGGCGTTTCCCCCTGGAAGCTCCCTCGTGCGCT |
| | CTCCTGTTCCGACCCTGCCGCTTACCGGATACCTGTCCGCCTTTCTCCCT |
| | TCGGGAAGCGTGGCGCTTTCTCATAGCTCACGCTGTAGGTATCTCAGTT |
| | CGGTGTAGGTCGTTCGCTCCAAGCTGGGCTGTGTGCACGAACCCCCCGT |
| | TCAGCCCGACCGCTGCGCCTTATCCGGTAACTATCGTCTTGAGTCCAAC |
| | CCGGTAAGACACGACTTATCGCCACTGGCAGCAGCCACTGGTAACAGG |
| | ATTAGCAGAGCGAGGTATGTAGGCGGTGCTACAGAGTTCTTGAAGTGG |
| | TGGCCTAACTACGGCTACACTAGAAGAACAGTATTTGGTATCTGCGCTC |
| | TGCTGAAGCCAGTTACCTTCGGAAAAAGAGTTGGTAGCTCTTGATCCGG |
| | CAAACAAACCACCGCTGGTAGCGGTGGTTTTTTTGTTTGCAAGCAGCAG |
| | ATTACGCGCAGAAAAAAAGGATCTCAAGAAGATCCTTTGATCTTTTCTA |
| | CGGGGTCTGACGCTCAGTGGAACGAAAACTCACGTTAAGGGATTTTGG |
| | TCATGAGATTATCAAAAAGGATCTTCACCTAGATCCTTTTAAATTAAAA |
| | ATGAAGTTTTAAATCAATCTAAAGTATATATGAGTAAACTTGGTCTGAC |
| | AGTTACCAATGCTTAATCAGTGAGGCACCTATCTCAGCGATCTGTCTAT |
| | TTCGTTCATCCATAGTTGCCTGACTCCCCGTCGTGTAGATAACTACGAT |
| | ACGGGAGGGCTTACCATCTGGCCCCAGTGCTGCAATGATACCGCGAGA |
| | CCCACGCTCACCGGCTCCAGATTTATCAGCAATAAACCAGCCAGCCGG |
| | AAGGGCCGAGCGCAGAAGTGGTCCTGCAACTTTATCCGCCTCCATCCAG |
| | TCTATTAATTGTTGCCGGGAAGCTAGAGTAAGTAGTTCGCCAGTTAATA |
| | GTTTGCGCAACGTTGTTGCCATTGCTACAGGCATCGTGGTGTCACGCTC |
| | GTCGTTTGGTATGGCTTCATTCAGCTCCGGTTCCCAACGATCAAGGCGA |
| | GTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGT |
| | TATGGCAGCACTGCATAATTCTCTTACTGTCATGCCATCCGTAAGATGC |
| | TTTTCTGTGACTGGTGAGTACTCAACCAAGTCATTCTGAGAATAGTGTA |
| | TGCGGCGACCGAGTTGCTCTTGCCCGGCGTCAATACGGGATAATACCGC |
| | GCCACATAGCAGAACTTTAAAAGTGCTCATCATTGGAAAACGTTCTTCG |
| | GGGCGAAAACTCTCAAGGATCTTACCGCTGTTGAGATCCAGTTCGATGT |
| | AACCCACTCGTGCACCCAACTGATCTTCAGCATCTTTTACTTTCACCAGC |
| | GTTTCTGGGTGAGCAAAAACAGGAAGGCAAAATGCCGCAAAAAAGGG |
| | AATAAGGGCGACACGGAAATGTTGAATACTCATACTCTTCCTTTTTCAA |
| | TATTATTGAAGCATTTATCAGGGTTATTGTCTCATGAGCGGATACATAT |
| | TTGAATGTATTTAGAAAAATAAACAAATAGGGGTTCCGCGCACATTTCC |
| | CCGAAAAGTGCCACCTAAATTGTAAGCGTTAATATTTTGTTAAAATTCG |
| | CGTTAAATTTTTGTTAAATCAGCTCATTTTTTAACCAATAGGCCGAAATC |
| | GGCAAAATCCCTTATAAATCAAAAGAATAGACCGAGATAGGGTTGAGT |
| | GTTGTTCCAGTTTGGAACAAGAGTCCACTATTAAAGAACGTGGACTCCA |
| | ACGTCAAAGGGCGAAAAACCGTCTATCAGGGCGATGGCCCACTACGTG |
| | AACCATCACCCTAATCAAGTTTTTTGGGGTCGAGGTGCCGTAAAGCACT |
| | AAATCGGAACCCTAAAGGGAGCCCCCGATTTAGAGCTTGACGGGGAAA |
| | GCCGGCGAACGTGGCGAGAAAGGAAGGGAAGAAAGCGAAAGGAGCGG |
| | GCGCTAGGGCGCTGGCAAGTGTAGCGGTCACGCTGCGCGTAACCACCA |
| | CACCCGCCGCGCTTAATGCGCCGCTACAGGGCGCGTCCCAT (SEQ ID |
| | NO: 6) |
| AAV6 | GGATCCGAATTCTTAATTAACATCATCAATAATATACCTTATTTTGGATT |
| | GAAGCCAATATGATAATGAGGGGGTGGAGTTTGTGACGTGGCGCGGGG |
| | CGTGGGAACGGGGCGGGTGACGTAGTAGTGTGGCGGAAGTGTGATGTT |
| | GCAAGTGTGGCGGAACACATGTAAGCGACGGATGTGGCAAAAGTGACG |
| | TTTTTGGTGTGCGCCGGTGTACACAGGAAGTGACAATTTTCGCGCGGTT |
| | TTAGGCGGATGTTGTAGTAAATTTGGGCGTAACCGAGTAAGATTTGGCC |
| | ATTTTCGCGGGAAAACTGAATAAGAGGAAGTGAAATCTGAATAATTTT |
| | GTGTTACTCATAGCGCGTAATATTTGTCTAGGGCCGCGGGGACTTTGAC |
| | CGTTTACGTGGAGACTCGCCCAGGTGTTTTTCTCAGGTGTTTTCCGCGTT |
| | CCGGGTCAAAGTTGGCGTTTTATTATTATAGTCAGGGGGATCCTCTAGA |
| | ACTAGTGGATCCGTCCCTAGAAGTAAAAAAGGGAAAAAAGAGTGTGTT |
| | TGTCAAAATAGGAGACAGGTGGTGGCAACCAGGGACTTATAGGGGACC |
| | TTACATCTACAGACCAACAGATGCCCCCTTACCATATACAGGAAGATAT |
| | GACTTAAATTGGGATAGGTGGGTCACAATCAACGGCTATAAAGTGTTAT |
| | ACAGATCCCTCCCCTTTCGTGAAAGACTCGCCAGAGCTAGACCTCCTTG |
| | GTGTATGCTAACTGAGAAAGAGAAAGACGACATGAAACAACAGGTACA |
| | TGATTATATTTATCTAGGAACAGGAATGCACTTTTGGGGAAAGGTTTTC |
| | CATACCAAGGAAGGGGCAGTGGCTGGACTGATAGAACATTATTCTGCA |
| | AAAACTTATGGTATGAGTTATTATGATTAGCCTTTATTTGCCCAACCTTG |
| | CGGTTCCCAGGGTTTAAATAAGTTTATGGTTACAAACTGTTCTTAAAAC |
| | AAGGATGTGAGACAAGTGGTTTCCTGAGTTGGTTTGGTATCAAATGTTC |
| | TGATCTGAGCTCTTAGTGTTCTATTTTCCTATGTTCTTTTGGAATCTATCC |
| | AAGTCTTATGTAAATGCTTATGTAAACCATAATATAAAAGAGTGCTGAT |
| | TTTTTGAGTAAACTTGCAACAGTCCTAACATTCTTCTCTCGTGTGTTTGT |
| | GTCTGTTCGCCATCCCGTCTCCGCTCGTCACTTATCCTTCACTTTTCAGA |
| | GGGTCCCCCCGCAGATCCCGGTCACCCTCAGGTCGGGACCTGCAGAAG |
| | ACGCCCGAGTGAGCACGCAGGGTCTCCATTTTGAAGCGGGAGGTTTGA |
| | ACGCGCAGCCGCCATGCCGGGGTTTTACGAGATTGTGATTAAGGTCCCC |
| | AGCGACCTTGACGAGCATCTGCCCGGCATTTCTGACAGCTTTGTGAACT |
| | GGGTGGCCGAGAAGGAATGGGAGTTGCCGCCAGATTCTGACATGGATC |
| | TGAATCTGATTGAGCAGGCACCCCTGACCGTGGCCGAGAAGCTGCAGC |
| | GCGACTTTCTGACGGAATGGCGCCGTGTGAGTAAGGCCCCGGAGGCCC |
| | TTTTCTTTGTGCAATTTGAGAAGGGAGAGAGCTACTTCCACATGCACGT |
| | GCTCGTGGAAACCACCGGGGTGAAATCCATGGTTTTGGGACGTTTCCTG |
| | AGTCAGATTCGCGAAAAACTGATTCAGAGAATTTACCGCGGGATCGAG |
| | CCGACTTTGCCAAACTGGTTCGCGGTCACAAAGACCAGAAATGGCGCC |
| | GGAGGCGGGAACAAGGTGGTGGATGAGTGCTACATCCCCAATTACTTG |
| | CTCCCCAAAACCCAGCCTGAGCTCCAGTGGGCGTGGACTAATATGGAA |
| | CAGTATTTAAGCGCCTGTTTGAATCTCACGGAGCGTAAACGGTTGGTGG |
| | CGCAGCATCTGACGCACGTGTCGCAGACGCAGGAGCAGAACAAAGAGA |
| | ATCAGAATCCCAATTCTGATGCGCCGGTGATCAGATCAAAAACTTCAGC |
| | CAGGTACATGGAGCTGGTCGGGTGGCTCGTGGACAAGGGGATTACCTC |
| | GGAGAAGCAGTGGATCCAGGAGGACCAGGCCTCATACATCTCCTTCAA |
| | TGCGGCCTCCAACTCGCGGTCCCAAATCAAGGCTGCCTTGGACAATGCG |
| | GGAAAGATTATGAGCCTGACTAAAACCGCCCCCGACTACCTGGTGGGC |
| | CAGCAGCCCGTGGAGGACATTTCCAGCAATCGGATTTATAAAATTTTGG |
| | AACTAAACGGGTACGATCCCCAATATGCGGCTTCCGTCTTTCTGGGATG |
| | GGCCACGAAAAAGTTCGGCAAGAGGAACACCATCTGGCTGTTTGGGCC |
| | TGCAACTACCGGGAAGACCAACATCGCGGAGGCCATAGCCCACACTGT |
| | GCCCTTCTACGGGTGCGTAAACTGGACCAATGAGAACTTTCCCTTCAAC |
| | GACTGTGTCGACAAGATGGTGATCTGGTGGGAGGAGGGGAAGATGACC |
| | GCCAAGGTCGTGGAGTCGGCCAAAGCCATTCTCGGAGGAAGCAAGGTG |
| | CGCGTGGACCAGAAATGCAAGTCCTCGGCCCAGATAGACCCGACTCCC |
| | GTGATCGTCACCTCCAACACCAACATGTGCGCCGTGATTGACGGGAACT |
| | CAACGACCTTCGAACACCAGCAGCCGTTGCAAGACCGGATGTTCAAAT |
| | TTGAACTCACCCGCCGTCTGGATCATGACTTTGGGAAGGTCACCAAGCA |
| | GGAAGTCAAAGACTTTTTCCGGTGGGCAAAGGATCACGTGGTTGAGGT |
| | GGAGCATGAATTCTACGTCAAAAAGGGTGGAGCCAAGAAAAGACCCGC |
| | CCCCAGTGACGCAGATATAAGTGAGCCCAAACGGGTGCGCGAGTCAGT |
| | TGCGCAGCCATCGACGTCAGACGCGGAAGCTTCGATCAACTACGCAGA |
| | CAGGTACCAAAACAAATGTTCTCGTCACGTGGGCATGAATCTGATGCTG |
| | TTTCCCTGCAGACAATGCGAGAGAATGAATCAGAATTCAAATATCTGCT |
| | TCACTCACGGACAGAAAGACTGTTTAGAGTGCTTTCCCGTGTCAGAATC |
| | TCAACCCGTTTCTGTCGTCAAAAAGGCGTATCAGAAACTGTGCTACATT |
| | CATCATATCATGGGAAAGGTGCCAGACGCTTGCACTGCCTGCGATCTGG |
| | TCAATGTGGATTTGGATGACTGCATCTTTGAACAATAAATGATTTAAAT |
| | CAGGTATGGCTGCCGATGGTTATCTTCCAGATTGGCTCGAGGACAACCT |
| | CTCTGAGGGCATTCGCGAGTGGTGGGACTTGAAACCTGGAGCCCCGAA |
| | ACCCAAAGCCAACCAGCAAAAGCAGGACGACGGCCGGGGTCTGGTGCT |
| | TCCTGGCTACAAGTACCTCGGACCCTTCAACGGACTCGACAAGGGGGA |
| | GCCCGTCAACGCGGCGGATGCAGCGGCCCTCGAGCACGACAAGGCCTA |
| | CGACCAGCAGCTCAAAGCGGGTGACAATCCGTACCTGCGGTATAACCA |
| | CGCCGACGCCGAGTTTCAGGAGCGTCTGCAAGAAGATACGTCTTTTGGG |
| | GGCAACCTCGGGCGAGCAGTCTTCCAGGCCAAGAAGAGGGTTCTCGAA |
| | CCTTTTGGTCTGGTTGAGGAAGGTGCTAAGACGGCTCCTGGAAAGAAA |
| | CGTCCGGTAGAGCAGTCGCCACAAGAGCCAGACTCCTCCTCGGGCATT |
| | GGCAAGACAGGCCAGCAGCCCGCTAAAAAGAGACTCAATTTTGGTCAG |
| | ACTGGCGACTCAGAGTCAGTCCCCGACCCACAACCTCTCGGAGAACCTC |
| | CAGCAACCCCCGCTGCTGTGGGACCTACTACAATGGCTTCAGGCGGTGG |
| | CGCACCAATGGCAGACAATAACGAAGGCGCCGACGGAGTGGGTAATGC |
| | CTCAGGAAATTGGCATTGCGATTCCACATGGCTGGGCGACAGAGTCATC |
| | ACCACCAGCACCCGAACATGGGCCTTGCCCACCTATAACAACCACCTCT |
| | ACAAGCAAATCTCCAGTGCTTCAACGGGGGCCAGCAACGACAACCACT |
| | ACTTCGGCTACAGCACCCCCTGGGGGTATTTTGATTTCAACAGATTCCA |
| | CTGCCATTTCTCACCACGTGACTGGCAGCGACTCATCAACAACAATTGG |
| | GGATTCCGGCCCAAGAGACTCAACTTCAAGCTCTTCAACATCCAAGTCA |
| | AGGAGGTCACGACGAATGATGGCGTCACGACCATCGCTAATAACCTTA |
| | CCAGCACGGTTCAAGTCTTCTCGGACTCGGAGTACCAGTTGCCGTACGT |
| | CCTCGGCTCTGCGCACCAGGGCTGCCTCCCTCCGTTCCCGGCGGACGTG |
| | TTCATGATTCCGCAGTACGGCTACCTAACGCTCAACAATGGCAGCCAGG |
| | CAGTGGGACGGTCATCCTTTTACTGCCTGGAATATTTCCCATCGCAGAT |
| | GCTGAGAACGGGCAATAACTTTACCTTCAGCTACACCTTCGAGGACGTG |
| | CCTTTCCACAGCAGCTACGCGCACAGCCAGAGCCTGGACCGGCTGATG |
| | AATCCTCTCATCGACCAGTACCTGTATTACCTGAACAGAACTCAGAATC |
| | AGTCCGGAAGTGCCCAAAACAAGGACTTGCTGTTTAGCCGGGGGTCTC |
| | CAGCTGGCATGTCTGTTCAGCCCAAAAACTGGCTACCTGGACCCTGTTA |
| | CCGGCAGCAGCGCGTTTCTAAAACAAAAACAGACAACAACAACAGCAA |
| | CTTTACCTGGACTGGTGCTTCAAAATATAACCTTAATGGGCGTGAATCT |
| | ATAATCAACCCTGGCACTGCTATGGCCTCACACAAAGACGACAAAGAC |
| | AAGTTCTTTCCCATGAGCGGTGTCATGATTTTTGGAAAGGAGAGCGCCG |
| | GAGCTTCAAACACTGCATTGGACAATGTCATGATCACAGACGAAGAGG |
| | AAATCAAAGCCACTAACCCCGTGGCCACCGAAAGATTTGGGACTGTGG |
| | CAGTCAATCTCCAGAGCAGCAGCACAGACCCTGCGACCGGAGATGTGC |
| | ATGTTATGGGAGCCTTACCTGGAATGGTGTGGCAAGACAGAGACGTAT |
| | ACCTGCAGGGTCCTATTTGGGCCAAAATTCCTCACACGGATGGACACTT |
| | TCACCCGTCTCCTCTCATGGGCGGCTTTGGACTTAAGCACCCGCCTCCTC |
| | AGATCCTCATCAAAAACACGCCTGTTCCTGCGAATCCTCCGGCAGAGTT |
| | TTCGGCTACAAAGTTTGCTTCATTCATCACCCAGTATTCCACAGGACAA |
| | GTGAGCGTGGAGATTGAATGGGAGCTGCAGAAAGAAAACAGCAAACG |
| | CTGGAATCCCGAAGTGCAGTATACATCTAACTATGCAAAATCTGCCAAC |
| | GTTGATTTCACTGTGGACAACAATGGACTTTATACTGAGCCTCGCCCCA |
| | TTGGCACCCGTTACCTCACCCGTCCCCTGTAATTGTCTGTTAATCAATAA |
| | ACCGGTTAATTCGTGTCAGTTGAACTTTGGTCTCATGTCGTTATTATCTT |
| | ATCTGGTCACCATAGCAATCGATGCATGTCCTTGGGTCCGGCCTGCTGA |
| | ATGCGCAGGCGGTCGGCCATGCCCCAGGCTTCGTTTTGACATCGGCGCA |
| | GGTCTTTGTAGTAGTCTTGCATGAGCCTTTCTACCGGCACTTCTTCTTCT |
| | CCTTCCTCTTGTCCTGCATCTCTTGCATCTATCGCTGCGGCGGCGGCGGA |
| | GTTTGGCCGTAGGTGGCGCCCTCTTCCTCCCATCGTGTGACCCCGAAGC |
| | CCCTCATCGGCTGAAGCAGGGCTAGGTCGGCGACAACGCGCTCGGCTA |
| | ATATGGCCTGCTGCACCTGCGTGAGGGTAGACTGGAAGTCATCCATGTC |
| | CACAAAGCGGTGGTATGCGCCCGTGTTGATGGTGTAAGTGCAGTTGGCC |
| | ATAACGGACCAGTTAACGGTCTGGTGACCCGGCTGCGAGAGCTCGGTG |
| | TACCTGAGACGCGAGTAAGCCCTCGAGTCAAATACGTAGTCGTTACAA |
| | GTCCGCACCAGGTACTGGTATCCCACCAAAAAGTGCGGCGGCGGCTGG |
| | CGGTAGAGGGGCCAGCGTAGGGTGGCCGGGGCTCCGGGGGCGAGATCT |
| | TCCAACATAAGGCGATGATATCCGTAGATGTACCTGGACATCCAGGTG |
| | ATGCCGGCGGCGGTGGTGGAGGCGCGCGGAAAGTCGCGGACGCGGTTC |
| | CAGATGTTGCGCAGCGGCAAAAAGTGCTCCATGGTCGGGACGCTCTGG |
| | CCGGTCAGGCGCGCGCAATCGTTGACGCTCTAGACCGTGCAAAAGGAG |
| | AGCCTGTAAGCGGGCACTCTTCCGTGGTCTGGTGGATAAATTCGCAAGG |
| | GTATCATGGCGGACGACCGGGGTTCGAGCCCCGTATCCGGCCGTCCGCC |
| | GTGATCCATGCGGTTACCGCCCGCGTGTCGAACCCAGGTGTGCGACGTC |
| | AGACAACGGGGGAGTGCTCCTTTTGGCTTCCTTCCAGGCGCGGCGGCTG |
| | CTGCGCTAGCTTTTTTGGCCACTGGCCGCGCGCAGCGTAAGCGGTTAGG |
| | CTGGAAAGCGAAAGCATTAAGTGGCTCGCTCCCTGTAGCCGGAGGGTT |
| | ATTTTCCAAGGGTTGAGTCGCGGGACCCCCGGTTCGAGTCTCGGACCGG |
| | CCGGACTGCGGCGAACGGGGGTTTGTCTCCCCGTCATGCAAGACCCCGC |
| | TTGCAAATTCCTCCGGAAACAGGGACGAGCCCCTTTTTTGCTTTTCCCA |
| | GATGCATCCGGTGCTGCGGCAGATGCGCCCCCCTCCTCAGCAGCGGCA |
| | AGAGCAAGAGCAGCGGCAGACATGCAGGGCACCCTCCCCTCCTCCTAC |
| | CGCGTCAGGAGGGGCGACATCCGCGGTTGACGCGGCAGCAGATGGTGA |
| | TTACGAACCCCCGCGGCGCCGGGCCCGGCACTACCTGGACTTGGAGGA |
| | GGGCGAGGGCCTGGCGCGGCTAGGAGCGCCCTCTCCTGAGCGGCACCC |
| | AAGGGTGCAGCTGAAGCGTGATACGCGTGAGGCGTACGTGCCGCGGCA |
| | GAACCTGTTTCGCGACCGCGAGGGAGAGGAGCCCGAGGAGATGCGGGA |
| | TCGAAAGTTCCACGCAGGGCGCGAGCTGCGGCATGGCCTGAATCGCGA |
| | GCGGTTGCTGCGCGAGGAGGACTTTGAGCCCGACGCGCGAACCGGGAT |
| | TAGTCCCGCGCGCGCACACGTGGCGGCCGCCGACCTGGTAACCGCATA |
| | CGAGCAGACGGTGAACCAGGGCGATCGCACCCTTTGGCGCATCCCATT |
| | CTCCAGTAACTTTATGTCCATGGGCGCACTCACAGACCTGGGCCAAAAC |
| | CTTCTCTACGCCAACTCCGCCCACGCGCTAGACATGACTTTTGAGGTGG |
| | ATCCCATGGACGAGCCCACCCTTCTTTATGTTTTGTTTGAAGTCTTTGAC |
| | GTGGTCCGTGTGCACCAGCCGCACCGCGGCGTCATCGAAACCGTGTACC |
| | TGCGCACGCCCTTCTCGGCCGGCAACGCCACAACATAAAGAAGCAAGC |
| | AACATCAACAACAGCTGCCGCCATGGGCTCCAGTGAGCAGGAACTGAA |
| | AGCCATTGTCAAAGATCTTGGTTGTGGGCCATATTTTTTGGGCACCTAT |
| | GACAAGCGCTTTCCAGGCTTTGTTTCTCCACACAAGCTCGCCTGCGCCA |
| | TAGTCAATACGGCCGGTCGCGAGACTGGGGGCGTACACTGGATGGCCT |
| | TTGCCTGGAACCCGCACTCAAAAACATGCTACCTCTTTGAGCCCTTTGG |
| | CTTTTCTGACCAGCGACTCAAGCAGGTTTACCAGTTTGAGTACGAGTCA |
| | CTCCTGCGCCGTAGCGCCATTGCTTCTTCCCCCGACCGCTGTATAACGCT |
| | GGAAAAGTCCACCCAAAGCGTACAGGGGCCCAACTCGGCCGCCTGTGG |
| | ACTATTCTGCTGCATGTTTCTCCACGCCTTTGCCAACTGGCCCCAAACTC |
| | CCATGGATCACAACCCCACCATGAACCTTATTACCGGGGTACCCAACTC |
| | CATGCTCAACAGTCCCCAGGTACAGCCCACCCTGCGTCGCAACCAGGA |
| | ACAGCTCTACAGCTTCCTGGAGCGCCACTCGCCCTACTTCCGCAGCCAC |
| | AGTGCGCAGATTAGGAGCGCCACTTCTTTTTGTCACTTGAAAAACATGT |
| | AAAAATAATGTACTAGAGACACTTTCAATAAAGGCAAATGCTTTTATTT |
| | GTACACTCTCGGGTGATTATTTACCCCCACCCTTGCCGTCTGCGCCGTTT |
| | AAAAATCAAAGGGGTTCTGCCGCGCATCGCTATGCGCCACTGGCAGGG |
| | ACACGTTGCGATACTGGTGTTTAGTGCTCCACTTAAACTCAGGCACAAC |
| | CATCCGCGGCAGCTCGGTGAAGTTTTCACTCCACAGGCTGCGCACCATC |
| | ACCAACGCGTTTAGCAGGTCGGGCGCCGATATCTTGAAGTCGCAGTTGG |
| | GGCCTCCGCCCTGCGCGCGCGAGTTGCGATACACAGGGTTGCAGCACT |
| | GGAACACTATCAGCGCCGGGTGGTGCACGCTGGCCAGCACGCTCTTGTC |
| | GGAGATCAGATCCGCGTCCAGGTCCTCCGCGTTGCTCAGGGCGAACGG |
| | AGTCAACTTTGGTAGCTGCCTTCCCAAAAAGGGCGCGTGCCCAGGCTTT |
| | GAGTTGCACTCGCACCGTAGTGGCATCAAAAGGTGACCGTGCCCGGTCT |
| | GGGCGTTAGGATACAGCGCCTGCATAAAAGCCTTGATCTGCTTAAAAG |
| | CCACCTGAGCCTTTGCGCCTTCAGAGAAGAACATGCCGCAAGACTTGCC |
| | GGAAAACTGATTGGCCGGACAGGCCGCGTCGTGCACGCAGCACCTTGC |
| | GTCGGTGTTGGAGATCTGCACCACATTTCGGCCCCACCGGTTCTTCACG |
| | ATCTTGGCCTTGCTAGACTGCTCCTTCAGCGCGCGCTGCCCGTTTTCGCT |
| | CGTCACATCCATTTCAATCACGTGCTCCTTATTTATCATAATGCTTCCGT |
| | GTAGACACTTAAGCTCGCCTTCGATCTCAGCGCAGCGGTGCAGCCACAA |
| | CGCGCAGCCCGTGGGCTCGTGATGCTTGTAGGTCACCTCTGCAAACGAC |
| | TGCAGGTACGCCTGCAGGAATCGCCCCATCATCGTCACAAAGGTCTTGT |
| | TGCTGGTGAAGGTCAGCTGCAACCCGCGGTGCTCCTCGTTCAGCCAGGT |
| | CTTGCATACGGCCGCCAGAGCTTCCACTTGGTCAGGCAGTAGTTTGAAG |
| | TTCGCCTTTAGATCGTTATCCACGTGGTACTTGTCCATCAGCGCGCGCG |
| | CAGCCTCCATGCCCTTCTCCCACGCAGACACGATCGGCACACTCAGCGG |
| | GTTCATCACCGTAATTTCACTTTCCGCTTCGCTGGGCTCTTCCTCTTCCT |
| | CTTGCGTCCGCATACCACGCGCCACTGGGTCGTCTTCATTCAGCCGCCG |
| | CACTGTGCGCTTACCTCCTTTGCCATGCTTGATTAGCACCGGTGGGTTGC |
| | TGAAACCCACCATTTGTAGCGCCACATCTTCTCTTTCTTCCTCGCTGTCC |
| | ACGATTACCTCTGGTGATGGCGGGCGCTCGGGCTTGGGAGAAGGGCGC |
| | TTCTTTTTCTTCTTGGGCGCAATGGCCAAATCCGCCGCCGAGGTCGATG |
| | GCCGCGGGCTGGGTGTGCGCGGCACCAGCGCGTCTTGTGATGAGTCTTC |
| | CTCGTCCTCGGACTCGATACGCCGCCTCATCCGCTTTTTTGGGGGCGCC |
| | CGGGGAGGCGGCGGCGACGGGGACGGGGACGACACGTCCTCCATGGTT |
| | GGGGGACGTCGCGCCGCACCGCGTCCGCGCTCGGGGGTGGTTTCGCGC |
| | TGCTCCTCTTCCCGACTGGCCATTTCCTTCTCCTATAGGCAGAAAAAGA |
| | TCATGGAGTCAGTCGAGAAGAAGGACAGCCTAACCGCCCCCTCTGAGT |
| | TCGCCACCACCGCCTCCACCGATGCCGCCAACGCGCCTACCACCTTCCC |
| | CGTCGAGGCACCCCCGCTTGAGGAGGAGGAAGTGATTATCGAGCAGGA |
| | CCCAGGTTTTGTAAGCGAAGACGACGAGGACCGCTCAGTACCAACAGA |
| | GGATAAAAAGCAAGACCAGGACAACGCAGAGGCAAACGAGGAACAAG |
| | TCGGGCGGGGGGACGAAAGGCATGGCGACTACCTAGATGTGGGAGACG |
| | ACGTGCTGTTGAAGCATCTGCAGCGCCAGTGCGCCATTATCTGCGACGC |
| | GTTGCAAGAGCGCAGCGATGTGCCCCTCGCCATAGCGGATGTCAGCCTT |
| | GCCTACGAACGCCACCTATTCTCACCGCGCGTACCCCCCAAACGCCAAG |
| | AAAACGGCACATGCGAGCCCAACCCGCGCCTCAACTTCTACCCCGTATT |
| | TGCCGTGCCAGAGGTGCTTGCCACCTATCACATCTTTTTCCAAAACTGC |
| | AAGATACCCCTATCCTGCCGTGCCAACCGCAGCCGAGCGGACAAGCAG |
| | CTGGCCTTGCGGCAGGGCGCTGTCATACCTGATATCGCCTCGCTCAACG |
| | AAGTGCCAAAAATCTTTGAGGGTCTTGGACGCGACGAGAAGCGCGCGG |
| | CAAACGCTCTGCAACAGGAAAACAGCGAAAATGAAAGTCACTCTGGAG |
| | TGTTGGTGGAACTCGAGGGTGACAACGCGCGCCTAGCCGTACTAAAAC |
| | GCAGCATCGAGGTCACCCACTTTGCCTACCCGGCACTTAACCTACCCCC |
| | CAAGGTCATGAGCACAGTCATGAGTGAGCTGATCGTGCGCCGTGCGCA |
| | GCCCCTGGAGAGGGATGCAAATTTGCAAGAACAAACAGAGGAGGGCCT |
| | ACCCGCAGTTGGCGACGAGCAGCTAGCGCGCTGGCTTCAAACGCGCGA |
| | GCCTGCCGACTTGGAGGAGCGACGCAAACTAATGATGGCCGCAGTGCT |
| | CGTTACCGTGGAGCTTGAGTGCATGCAGCGGTTCTTTGCTGACCCGGAG |
| | ATGCAGCGCAAGCTAGAGGAAACATTGCACTACACCTTTCGACAGGGC |
| | TACGTACGCCAGGCCTGCAAGATCTCCAACGTGGAGCTCTGCAACCTGG |
| | TCTCCTACCTTGGAATTTTGCACGAAAACCGCCTTGGGCAAAACGTGCT |
| | TCATTCCACGCTCAAGGGCGAGGCGCGCCGCGACTACGTCCGCGACTG |
| | CGTTTACTTATTTCTATGCTACACCTGGCAGACGGCCATGGGCGTTTGG |
| | CAGCAGTGCTTGGAGGAGTGCAACCTCAAGGAGCTGCAGAAACTGCTA |
| | AAGCAAAACTTGAAGGACCTATGGACGGCCTTCAACGAGCGCTCCGTG |
| | GCCGCGCACCTGGCGGACATCATTTTCCCCGAACGCCTGCTTAAAACCC |
| | TGCAACAGGGTCTGCCAGACTTCACCAGTCAAAGCATGTTGCAGAACTT |
| | TAGGAACTTTATCCTAGAGCGCTCAGGAATCTTGCCCGCCACCTGCTGT |
| | GCACTTCCTAGCGACTTTGTGCCCATTAAGTACCGCGAATGCCCTCCGC |
| | CGCTTTGGGGCCACTGCTACCTTCTGCAGCTAGCCAACTACCTTGCCTA |
| | CCACTCTGACATAATGGAAGACGTGAGCGGTGACGGTCTACTGGAGTG |
| | TCACTGTCGCTGCAACCTATGCACCCCGCACCGCTCCCTGGTTTGCAAT |
| | TCGCAGCTGCTTAACGAAAGTCAAATTATCGGTACCTTTGAGCTGCAGG |
| | GTCCCTCGCCTGACGAAAAGTCCGCGGCTCCGGGGTTGAAACTCACTCC |
| | GGGGCTGTGGACGTCGGCTTACCTTCGCAAATTTGTACCTGAGGACTAC |
| | CACGCCCACGAGATTAGGTTCTACGAAGACCAATCCCGCCCGCCTAATG |
| | CGGAGCTTACCGCCTGCGTCATTACCCAGGGCCACATTCTTGGCCAATT |
| | GCAAGCCATCAACAAAGCCCGCCAAGAGTTTCTGCTACGAAAGGGACG |
| | GGGGGTTTACTTGGACCCCCAGTCCGGCGAGGAGCTCAACCCAATCCCC |
| | CCGCCGCCGCAGCCCTATCAGCAGCAGCCGCGGGCCCTTGCTTCCCAGG |
| | ATGGCACCCAAAAAGAAGCTGCAGCTGCCGCCGCCACCCACGGACGAG |
| | GAGGAATACTGGGACAGTCAGGCAGAGGAGGTTTTGGACGAGGAGGA |
| | GGAGGACATGATGGAAGACTGGGAGAGCCTAGACGAGGAAGCTTCCG |
| | AGGTCGAAGAGGTGTCAGACGAAACACCGTCACCCTCGGTCGCATTCC |
| | CCTCGCCGGCGCCCCAGAAATCGGCAACCGGTTCCAGCATGGCTACAA |
| | CCTCCGCTCCTCAGGCGCCGCCGGCACTGCCCGTTCGCCGACCCAACCG |
| | TAGATGGGACACCACTGGAACCAGGGCCGGTAAGTCCAAGCAGCCGCC |
| | GCCGTTAGCCCAAGAGCAACAACAGCGCCAAGGCTACCGCTCATGGCG |
| | CGGGCACAAGAACGCCATAGTTGCTTGCTTGCAAGACTGTGGGGGCAA |
| | CATCTCCTTCGCCCGCCGCTTTCTTCTCTACCATCACGGCGTGGCCTTCC |
| | CCCGTAACATCCTGCATTACTACCGTCATCTCTACAGCCCATACTGCAC |
| | CGGCGGCAGCGGCAGCAACAGCAGCGGCCACACAGAAGCAAAGGCGA |
| | CCGGATAGCAAGACTCTGACAAAGCCCAAGAAATCCACAGCGGCGGCA |
| | GCAGCAGGAGGAGGAGCGCTGCGTCTGGCGCCCAACGAACCCGTATCG |
| | ACCCGCGAGCTTAGAAACAGGATTTTTCCCACTCTGTATGCTATATTTC |
| | AACAGAGCAGGGGCCAAGAACAAGAGCTGAAAATAAAAAACAGGTCT |
| | CTGCGATCCCTCACCCGCAGCTGCCTGTATCACAAAAGCGAAGATCAGC |
| | TTCGGCGCACGCTGGAAGACGCGGAGGCTCTCTTCAGTAAATACTGCGC |
| | GCTGACTCTTAAGGACTAGTTTCGCGCCCTTTCTCAAATTTAAGCGCGA |
| | AAACTACGTCATCTCCAGCGGCCACACCCGGCGCCAGCACCTGTTGTCA |
| | GCGCCATTATGAGCAAGGAAATTCCCACGCCCTACATGTGGAGTTACCA |
| | GCCACAAATGGGACTTGCGGCTGGAGCTGCCCAAGACTACTCAACCCG |
| | AATAAACTACATGAGCGCGGGACCCCACATGATATCCCGGGTCAACGG |
| | AATACGCGCCCACCGAAACCGAATTCTCCTGGAACAGGCGGCTATTAC |
| | CACCACACCTCGTAATAACCTTAATCCCCGTAGTTGGCCCGCTGCCCTG |
| | GTGTACCAGGAAAGTCCTGCTCCCACCACTGTGGTACTTCCCAGAGACG |
| | CCCAGGCCGAAGTTCAGATGACTAACTCAGGGGCGCAGCTTGCGGGCG |
| | GCTTTCGTCACAGGGTGCGGTCGCCCGGGCAGGGTATAACTCACCTGAC |
| | AATCAGAGGGCGAGGTATTCAGCTCAACGACGAGTCGGTGAGCTCCTC |
| | GCTTGGTCTCCGTCCGGACGGGACATTTCAGATCGGCGGCGCCGGCCGC |
| | TCTTCATTCACGCCTCGTCAGGCAATCCTAACTCTGCAGACCTCGTCCTC |
| | TGAGCCGCGCTCTGGAGGCATTGGAACTCTGCAATTTATTGAGGAGTTT |
| | GTGCCATCGGTCTACTTTAACCCCTTCTCGGGACCTCCCGGCCACTATCC |
| | GGATCAATTTATTCCTAACTTTGACGCGGTAAAGGACTCGGCGGACGGC |
| | TACGACTGAATGTTAAGTGGAGAGGCAGAGCAACTGCGCCTGAAACAC |
| | CTGGTCCACTGTCGCCGCCACAAGTGCTTTGCCCGCGACTCCGGTGAGT |
| | TTTGCTACTTTGAATTGCCCGAGGATCATATCGAGGGCCCGGCGCACGG |
| | CGTCCGGCTTACCGCCCAGGGAGAGCTTGCCCGTAGCCTGATTCGGGAG |
| | TTTACCCAGCGCCCCCTGCTAGTTGAGCGGGACAGGGGACCCTGTGTTC |
| | TCACTGTGATTTGCAACTGTCCTAACCCTGGATTACATCAAGATCTTTGT |
| | TGCCATCTCTGTGCTGAGTATAATAAATACAGAAATTAAAATATACTGG |
| | GGCTCCTATCGCCATCCTGTAAACGCCACCGTCTTCACCCGCCCAAGCA |
| | AACCAAGGCGAACCTTACCTGGTACTTTTAACATCTCTCCCTCTGTGATT |
| | TACAACAGTTTCAACCCAGACGGAGTGAGTCTACGAGAGAACCTCTCC |
| | GAGCTCAGCTACTCCATCAGAAAAAACACCACCCTCCTTACCTGCCGGG |
| | AACGTACGAGTGCGTCACCGGCCGCTGCACCACACCTACCGCCTGACC |
| | GTAAACCAGACTTTTTCCGGACAGACCTCAATAACTCTGTTTACCAGAA |
| | CAGGAGGTGAGCTTAGAAAACCCTTAGGGTATTAGGCCAAAGGCGCAG |
| | CTACTGTGGGGTTTATGAACAATTCAAGCAACTCTACGGGCTATTCTAA |
| | TTCAGGTTTCTCTAGAAATGGACGGAATTATTACAGAGCAGCGCCTGCT |
| | AGAAAGACGCAGGGCAGCGGCCGAGCAACAGCGCATGAATCAAGAGC |
| | TCCAAGACATGGTTAACTTGCACCAGTGCAAAAGGGGTATCTTTTGTCT |
| | GGTAAAGCAGGCCAAAGTCACCTACGACAGTAATACCACCGGACACCG |
| | CCTTAGCTACAAGTTGCCAACCAAGCGTCAGAAATTGGTGGTCATGGTG |
| | GGAGAAAAGCCCATTACCATAACTCAGCACTCGGTAGAAACCGAAGGC |
| | TGCATTCACTCACCTTGTCAAGGACCTGAGGATCTCTGCACCCTTATTA |
| | AGACCCTGTGCGGTCTCAAAGATCTTATTCCCTTTAACTAATAAAAAAA |
| | AATAATAAAGCATCACTTACTTAAAATCAGTTAGCAAATTTCTGTCCAG |
| | TTTATTCAGCAGCACCTCCTTGCCCTCCTCCCAGCTCTGGTATTGCAGCT |
| | TCCTCCTGGCTGCAAACTTTCTCCACAATCTAAATGGAATGTCAGTTTCC |
| | TCCTGTTCCTGTCCATCCGCACCCACTATCTTCATGTTGTTGCAGATGAA |
| | GCGCGCAAGACCGTCTGAAGATACCTTCAACCCCGTGTATCCATATGAC |
| | ACGGAAACCGGTCCTCCAACTGTGCCTTTTCTTACTCCTCCCTTTGTATC |
| | CCCCAATGGGTTTCAAGAGAGTCCCCCTGGGGTACTCTCTTTGCGCCTA |
| | TCCGAACCTCTAGTTACCTCCAATGGCATGCTTGCGCTCAAAATGGGCA |
| | ACGGCCTCTCTCTGGACGAGGCCGGCAACCTTACCTCCCAAAATGTAAC |
| | CACTGTGAGCCCACCTCTCAAAAAAACCAAGTCAAACATAAACCTGGA |
| | AATATCTGCACCCCTCACAGTTACCTCAGAAGCCCTAACTGTGGCTGCC |
| | GCCGCACCTCTAATGGTCGCGGGCAACACACTCACCATGCAATCACAG |
| | GCCCCGCTAACCGTGCACGACTCCAAACTTAGCATTGCCACCCAAGGAC |
| | CCCTCACAGTGTCAGAAGGAAAGCTAGCCCTGCAAACATCAGGCCCCC |
| | TCACCACCACCGATAGCAGTACCCTTACTATCACTGCCTCACCCCCTCT |
| | AACTACTGCCACTGGTAGCTTGGGCATTGACTTGAAAGAGCCCATTTAT |
| | ACACAAAATGGAAAACTAGGACTAAAGTACGGGGCTCCTTTGCATGTA |
| | ACAGACGACCTAAACACTTTGACCGTAGCAACTGGTCCAGGTGTGACT |
| | ATTAATAATACTTCCTTGCAAACTAAAGTTACTGGAGCCTTGGGTTTTG |
| | ATTCACAAGGCAATATGCAACTTAATGTAGCAGGAGGACTAAGGATTG |
| | ATTCTCAAAACAGACGCCTTATACTTGATGTTAGTTATCCGTTTGATGCT |
| | CAAAACCAACTAAATCTAAGACTAGGACAGGGCCCTCTTTTTATAAACT |
| | CAGCCCACAACTTGGATATTAACTACAACAAAGGCCTTTACTTGTTTAC |
| | AGCTTCAAACAATTCCAAAAAGCTTGAGGTTAACCTAAGCACTGCCAA |
| | GGGGTTGATGTTTGACGCTACAGCCATAGCCATTAATGCAGGAGATGG |
| | GCTTGAATTTGGTTCACCTAATGCACCAAACACAAATCCCCTCAAAACA |
| | AAAATTGGCCATGGCCTAGAATTTGATTCAAACAAGGCTATGGTTCCTA |
| | AACTAGGAACTGGCCTTAGTTTTGACAGCACAGGTGCCATTACAGTAGG |
| | AAACAAAAATAATGATAAGCTAACTTTGTGGACCACACCAGCTCCATCT |
| | CCTAACTGTAGACTAAATGCAGAGAAAGATGCTAAACTCACTTTGGTCT |
| | TAACAAAATGTGGCAGTCAAATACTTGCTACAGTTTCAGTTTTGGCTGT |
| | TAAAGGCAGTTTGGCTCCAATATCTGGAACAGTTCAAAGTGCTCATCTT |
| | ATTATAAGATTTGACGAAAATGGAGTGCTACTAAACAATTCCTTCCTGG |
| | ACCCAGAATATTGGAACTTTAGAAATGGAGATCTTACTGAAGGCACAG |
| | CCTATACAAACGCTGTTGGATTTATGCCTAACCTATCAGCTTATCCAAA |
| | ATCTCACGGTAAAACTGCCAAAAGTAACATTGTCAGTCAAGTTTACTTA |
| | AACGGAGACAAAACTAAACCTGTAACACTAACCATTACACTAAACGGT |
| | ACACAGGAAACAGGAGACACAACTCCAAGTGCATACTCTATGTCATTTT |
| | CATGGGACTGGTCTGGCCACAACTACATTAATGAAATATTTGCCACATC |
| | CTCTTACACTTTTTCATACATTGCCCAAGAATAAAGAATCGTTTGTGTTA |
| | TGTTTCAACGTGTTTATTTTTCAATTGCAGAAAATTTCAAGTCATTTTTC |
| | ATTCAGTAGTATAGCCCCACCACCACATAGCTTATACAGATCACCGTAC |
| | CTTAATCAAACTCACAGAACCCTAGTATTCAACCTGCCACCTCCCTCCC |
| | AACACACAGAGTACACAGTCCTTTCTCCCCGGCTGGCCTTAAAAAGCAT |
| | CATATCATGGGTAACAGACATATTCTTAGGTGTTATATTCCACACGGTT |
| | TCCTGTCGAGCCAAACGCTCATCAGTGATATTAATAAACTCCCCGGGCA |
| | GCTCACTTAAGTTCATGTCGCTGTCCAGCTGCTGAGCCACAGGCTGCTG |
| | TCCAACTTGCGGTTGCTTAACGGGCGGCGAAGGAGAAGTCCACGCCTA |
| | CATGGGGGTAGAGTCATAATCGTGCATCAGGATAGGGCGGTGGTGCTG |
| | CAGCAGCGCGCGAATAAACTGCTGCCGCCGCCGCTCCGTCCTGCAGGA |
| | ATACAACATGGCAGTGGTCTCCTCAGCGATGATTCGCACCGCCCGCAGC |
| | ATAAGGCGCCTTGTCCTCCGGGCACAGCAGCGCACCCTGATCTCACTTA |
| | AATCAGCACAGTAACTGCAGCACAGCACCACAATATTGTTCAAAATCC |
| | CACAGTGCAAGGCGCTGTATCCAAAGCTCATGGCGGGGACCACAGAAC |
| | CCACGTGGCCATCATACCACAAGCGCAGGTAGATTAAGTGGCGACCCC |
| | TCATAAACACGCTGGACATAAACATTACCTCTTTTGGCATGTTGTAATT |
| | CACCACCTCCCGGTACCATATAAACCTCTGATTAAACATGGCGCCATCC |
| | ACCACCATCCTAAACCAGCTGGCCAAAACCTGCCCGCCGGCTATACACT |
| | GCAGGGAACCGGGACTGGAACAATGACAGTGGAGAGCCCAGGACTCGT |
| | AACCATGGATCATCATGCTCGTCATGATATCAATGTTGGCACAACACAG |
| | GCACACGTGCATACACTTCCTCAGGATTACAAGCTCCTCCCGCGTTAGA |
| | ACCATATCCCAGGGAACAACCCATTCCTGAATCAGCGTAAATCCCACAC |
| | TGCAGGGAAGACCTCGCACGTAACTCACGTTGTGCATTGTCAAAGTGTT |
| | ACATTCGGGCAGCAGCGGATGATCCTCCAGTATGGTAGCGCGGGTTTCT |
| | GTCTCAAAAGGAGGTAGACGATCCCTACTGTACGGAGTGCGCCGAGAC |
| | AACCGAGATCGTGTTGGTCGTAGTGTCATGCCAAATGGAACGCCGGAC |
| | GTAGTCATATTTCCTGAAGCAAAACCAGGTGCGGGCGTGACAAACAGA |
| | TCTGCGTCTCCGGTCTCGCCGCTTAGATCGCTCTGTGTAGTAGTTGTAGT |
| | ATATCCACTCTCTCAAAGCATCCAGGCGCCCCCTGGCTTCGGGTTCTAT |
| | GTAAACTCCTTCATGCGCCGCTGCCCTGATAACATCCACCACCGCAGAA |
| | TAAGCCACACCCAGCCAACCTACACATTCGTTCTGCGAGTCACACACGG |
| | GAGGAGCGGGAAGAGCTGGAAGAACCATGTTTTTTTTTTTATTCCAAAA |
| | GATTATCCAAAACCTCAAAATGAAGATCTATTAAGTGAACGCGCTCCCC |
| | TCCGGTGGCGTGGTCAAACTCTACAGCCAAAGAACAGATAATGGCATT |
| | TGTAAGATGTTGCACAATGGCTTCCAAAAGGCAAACGGCCCTCACGTCC |
| | AAGTGGACGTAAAGGCTAAACCCTTCAGGGTGAATCTCCTCTATAAAC |
| | ATTCCAGCACCTTCAACCATGCCCAAATAATTCTCATCTCGCCACCTTCT |
| | CAATATATCTCTAAGCAAATCCCGAATATTAAGTCCGGCCATTGTAAAA |
| | ATCTGCTCCAGAGCGCCCTCCACCTTCAGCCTCAAGCAGCGAATCATGA |
| | TTGCAAAAATTCAGGTTCCTCACAGACCTGTATAAGATTCAAAAGCGGA |
| | ACATTAACAAAAATACCGCGATCCCGTAGGTCCCTTCGCAGGGCCAGCT |
| | GAACATAATCGTGCAGGTCTGCACGGACCAGCGCGGCCACTTCCCCGC |
| | CAGGAACCATGACAAAAGAACCCACACTGATTATGACACGCATACTCG |
| | GAGCTATGCTAACCAGCGTAGCCCCGATGTAAGCTTGTTGCATGGGCGG |
| | CGATATAAAATGCAAGGTGCTGCTCAAAAAATCAGGCAAAGCCTCGCG |
| | CAAAAAAGAAAGCACATCGTAGTCATGCTCATGCAGATAAAGGCAGGT |
| | AAGCTCCGGAACCACCACAGAAAAAGACACCATTTTTCTCTCAAACAT |
| | GTCTGCGGGTTTCTGCATAAACACAAAATAAAATAACAAAAAAACATT |
| | TAAACATTAGAAGCCTGTCTTACAACAGGAAAAACAACCCTTATAAGC |
| | ATAAGACGGACTACGGCCATGCCGGCGTGACCGTAAAAAAACTGGTCA |
| | CCGTGATTAAAAAGCACCACCGACAGCTCCTCGGTCATGTCCGGAGTCA |
| | TAATGTAAGACTCGGTAAACACATCAGGTTGATTCACATCGGTCAGTGC |
| | TAAAAAGCGACCGAAATAGCCCGGGGGAATACATACCCGCAGGCGTAG |
| | AGACAACATTACAGCCCCCATAGGAGGTATAACAAAATTAATAGGAGA |
| | GAAAAACACATAAACACCTGAAAAACCCTCCTGCCTAGGCAAAATAGC |
| | ACCCTCCCGCTCCAGAACAACATACAGCGCTTCCACAGCGGCAGCCAT |
| | AACAGTCAGCCTTACCAGTAAAAAAGAAAACCTATTAAAAAAACACCA |
| | CTCGACACGGCACCAGCTCAATCAGTCACAGTGTAAAAAAGGGCCAAG |
| | TGCAGAGCGAGTATATATAGGACTAAAAAATGACGTAACGGTTAAAGT |
| | CCACAAAAAACACCCAGAAAACCGCACGCGAACCTACGCCCAGAAACG |
| | AAAGCCAAAAAACCCACAACTTCCTCAAATCGTCACTTCCGTTTTCCCA |
| | CGTTACGTCACTTCCCATTTTAAGAAAACTACAATTCCCAACACATACA |
| | AGTTACTCCGCCCTAAAACCTACGTCACCCGCCCCGTTCCCACGCCCCG |
| | CGCCACGTCACAAACTCCACCCCCTCATTATCATATTGGCTTCAATCCA |
| | AAATAAGGTATATTATTGATGATGTTAATTAAGAATTCGGATCTGCGAC |
| | GCGAGGCTGGATGGCCTTCCCCATTATGATTCTTCTCGCTTCCGGCGGC |
| | ATCGGGATGCCCGCGTTGCAGGCCATGCTGTCCAGGCAGGTAGATGAC |
| | GACCATCAGGGACAGCTTCAAGGCCAGCAAAAGGCCAGGAACCGTAAA |
| | AAGGCCGCGTTGCTGGCGTTTTTCCATAGGCTCCGCCCCCCTGACGAGC |
| | ATCACAAAAATCGACGCTCAAGTCAGAGGTGGCGAAACCCGACAGGAC |
| | TATAAAGATACCAGGCGTTTCCCCCTGGAAGCTCCCTCGTGCGCTCTCC |
| | TGTTCCGACCCTGCCGCTTACCGGATACCTGTCCGCCTTTCTCCCTTCGG |
| | GAAGCGTGGCGCTTTCTCATAGCTCACGCTGTAGGTATCTCAGTTCGGT |
| | GTAGGTCGTTCGCTCCAAGCTGGGCTGTGTGCACGAACCCCCCGTTCAG |
| | CCCGACCGCTGCGCCTTATCCGGTAACTATCGTCTTGAGTCCAACCCGG |
| | TAAGACACGACTTATCGCCACTGGCAGCAGCCACTGGTAACAGGATTA |
| | GCAGAGCGAGGTATGTAGGCGGTGCTACAGAGTTCTTGAAGTGGTGGC |
| | CTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAA |
| | ACAAACCACCGCTGGTAGCGGTGGTTTTTTTGTTTGCAAGCAGCAGATT |
| | ACGCGCAGAAAAAAAGGATCTCAAGAAGATCCTTTGATCTTTTCTACGG |
| | GGTCTGACGCTCAGTGGAACGAAAACTCACGTTAAGGGATTTTGGTCAT |
| | GAGATTATCAAAAAGGATCTTCACCTAGATCCTTTTAAATCAATCTAAA |
| | GTATATATGAGTAAACTTGGTCTGACAGTTACCAATGCTTAATCAGTGA |
| | GGCACCTATCTCAGCGATCTGTCTATTTCGTTCATCCATAGTTGCCTGAC |
| | TCCCCGTCGTGTAGATAACTACGATACGGGAGGGCTTACCATCTGGCCC |
| | CAGTGCTGCAATGATACCGCGAGACCCACGCTCACCGGCTCCAGATTTA |
| | TCAGCAATAAACCAGCCAGCCGGAAGGGCCGAGCGCAGAAGTGGTCCT |
| | GCAACTTTATCCGCCTCCATCCAGTCTATTAATTGTTGCCGGGAAGCTA |
| | GAGTAAGTAGTTCGCCAGTTAATAGTTTGCGCAACGTTGTTGCCATTGC |
| | TACAGGCATCGTGGTGTCACGCTCGTCGTTTGGTATGGCTTCATTCAGC |
| | TCCGGTTCCCAACGATCAAGGCGAGTTACATGATCCCCCATGTTGTGCA |
| | AAAAAGCGGTTAGCTCCTTCGGTCCTCCGATCGTTGTCAGAAGTAAGTT |
| | GGCCGCAGTGTTATCACTCATGGTTATGGCAGCACTGCATAATTCTCTT |
| | ACTGTCATGCCATCCGTAAGATGCTTTTCTGTGACTGGTGAGTACTCAA |
| | CCAAGTCATTCTGAGAATAGTGTATGCGGCGACCGAGTTGCTCTTGCCC |
| | GGCGTCAACACGGGATAATACCGCGCCACATAGCAGAACTTTAAAAGT |
| | GCTCATCATTGGAAAACGTTCTTCGGGGCGAAAACTCTCAAGGATCTTA |
| | CCGCTGTTGAGATCCAGTTCGATGTAACCCACTCGTGCACCCAACTGAT |
| | CTTCAGCATCTTTTACTTTCACCAGCGTTTCTGGGTGAGCAAAAACAGG |
| | AAGGCAAAATGCCGCAAAAAAGGGAATAAGGGCGACACGGAAATGTT |
| | GAATACTCATACTCTTCCTTTTTCAATATTATTGAAGCATTTATCAGGGT |
| | TATTGTCTCATGAGCGGATACATATTTGAATGTATTTAGAAAAATAAAC |
| | AAATAGGGGTTCCGCGCACATTTCCCCGAAAAGTGCCACCTGACGTCTA |
| | AGAAACCATTATTATCATGACATTAACCTATAAAAATAGGCGTATCACG |
| | AGGCCCTTTCGTCTTCAAGAATT (SEQ ID NO: 7) |

In one embodiment, the adeno-associated viral vector is Anc80L65 or Anc80. In one embodiment, the adeno-associated viral vector (e.g., Anc80L65 or Anc80) is administered to at-risk tissue by aerosolization of a composition comprising an Anc80 viral vector that codes for the expression of acid ceramidase. Methods of administration also include intra-tracheal injection. In one embodiment, the adeno-associated viral vector includes a nucleotide sequence selected from any one of SEQ ID NO: 1, SEQ ID NO: 2, SEQ ID NO: 3, SEQ ID NO: 4, SEQ ID NO: 5, SEQ ID NO: 6, or SEQ ID NO: 7.

Methods and compositions for in vivo delivery of a construct that expresses a sphingolipid-metabolizing protein such as ceramidase were explored. For applications where more sustained expression of a sphingolipid metabolizing enzyme is required, expression from an adeno-associated viral vector such as Anc80L65 and/or Anc80 vector may be desirable.

Adeno-associated viruses have emerged as one of the most promising vectors in the field of gene therapy. Preclinical and clinical studies have validated the use of adeno-associated viral vectors (AAVs) as a safe and efficient delivery vehicle for gene transfer. AAV vectors are known to be expressed for several months or longer post administration; thus, they provide a more extensive time frame than modRNA.

The vector may be administered by introducing into the cell a nucleic acid molecule that encodes the ceramidase (either active ceramidase or ceramidase precursor protein, as described above) (JOSEPH SAMBROOK & DAVID W. RUSSELL, 1-3 MOLECULAR CLONING: A LABORATORY MANUAL (3d ed. 2001); SHORT PROTOCOLS IN MOLECULAR BIOLOGY (Frederick M. Ausubel et al. eds., 1999); U.S. Pat. No. 4,237,224 to Cohen & Boyer; each of which is hereby incorporated by reference in its entirety).

Nucleic acid agents for use in the methods of the present disclosure can be delivered to a cell in a number of ways known in the art. Transfection is a procedure that introduces foreign nucleic acids into cells to produce genetically modified cells. The genetic modifications can be transient or permanent. The delivery of a nucleic acid can mean the transfection of a nucleic acid. For example, the nucleic acid can be contained within a vector, e.g., a vector that can be transferred to the cell(s) and provide for expression of the nucleic acid therein. Such vectors include chromosomal vectors (e.g., artificial chromosomes), non-chromosomal vectors, and synthetic nucleic acids. Vectors include plasmids, viruses, and phages, such as retroviral vectors, lentiviral vectors, adenoviral vectors, bacteria, bacteriophage, erythrocyte ghosts, herpes viral vectors, baculoviral vectors, exosomes, virus-like particles (VLPs), and adeno-associated vectors.

Nucleic acid agents can be transfected into the cell(s) using ex vivo methods, as will be apparent to the skilled artisan. For example, nucleic acids and vectors can be delivered to cells by physical means, e.g., by electroporation, lipids, cationic lipids, liposomes, DNA gun, DNA biolistics, calcium phosphate precipitation, injection, or delivery of naked nucleic acid.

As an alternative to non-infective delivery of nucleic acids as described above, naked DNA or infective transformation vectors can be used for delivery, whereby the naked DNA or infective transformation vector contains a recombinant gene that encodes the acid ceramidase/acid ceramidase precursor protein. The nucleic acid molecule is then expressed in the transformed cell.

The recombinant gene includes, operatively coupled to one another, an upstream promoter operable in the cell in which the gene is to be expressed and optionally other suitable regulatory elements (i.e., enhancer or inducer elements), a coding sequence that encodes the nucleic acid, and a downstream transcription termination region. Any suitable constitutive promoter or inducible promoter can be used to regulate transcription of the recombinant gene, and one of skill in the art can readily select and utilize such promoters, whether now known or hereafter developed. The promoter can also be specific for expression in the cell(s) whose survival is to be promoted. Tissue specific promoters can also be made inducible/repressible using, e.g., a TetO response element. Other inducible elements can also be used. Known recombinant techniques can be utilized to prepare the recombinant gene, transfer it into the expression vector (if used), and administer the vector or naked DNA to the cell. Exemplary procedures are described in SAMBROOK & RUSSELL, 1-3 MOLECULAR CLONING: A LABORATORY MANUAL (3d ed. 2001), which is hereby incorporated by reference in its entirety. One of skill in the art can readily modify these procedures, as desired, using known variations of the procedures described therein.

Any suitable viral or infective transformation vector may be used, preferably an adeno-associated virus. Other exemplary viral vectors include, without limitation, adenovirus, and retroviral vectors (including lentiviral vectors).

Adeno-associated viral gene delivery vehicles can be constructed and used to deliver into cells a recombinant gene encoding a desired nucleic acid. The use of adeno-associated viral gene delivery vehicles in vitro is described in Chatterjee et al, “Dual-target Inhibition of HIV-1 in Vitro by Means of an Adeno-associated Virus Antisense Vector,” Science 258:1485-8 (1992); Walsh et al, “Regulated High Level Expression of a Human γ-Globin Gene Introduced into Erythroid Cells by an Adeno-associated Virus Vector,” Proc. Nat'l. Acad. Sci. USA 89:7257-61 (1992); Walsh et al, “Phenotypic Correction of Fanconi Anemia in Human Hematopoietic Cells with a Recombinant Adeno-associated Virus Vector,” J. Clin. Invest. 94:1440-8 (1994); Flotte et al., “Expression of the Cystic Fibrosis Transmembrane Conductance Regulator from a Novel Adeno-associated Virus Promoter,” J. Biol. Chem. 268:3781-90 (1993); Ponnazhagan et al., “Suppression of Human a-Globin Gene Expression Mediated by the Recombinant Adeno-associated Virus 2-based Antisense Vectors,” J. Exp. Med. 179:733-8 (1994); Miller et al., “Recombinant Adeno-associated Virus (rAAV)-mediated Expression of a Human γ-Globin Gene in Human Progenitor-derived Erythroid Cells,” Proc. Nat'l. Acad. Sci. USA 91:10183-7 (1994); Einerhand et al, “Regulated High-level Human β-Globin Gene Expression in Erythroid Cells Following Recombinant Adeno-associated Virus-mediated Gene Transfer,” Gene Ther. 2:336-43 (1995); Luo et al, “Adeno-associated Virus 2-mediated Gene Transfer and Functional Expression of the Human Granulocyte-macrophage Colony-stimulating Factor,” Exp. Hematol. 23: 1261-7 (1995); and Zhou et al, “Adeno-associated Virus 2-mediated Transduction and Erythroid Cell-specific Expression of a Human β-Globin Gene,” Gene Ther. 3:223-9 (1996), each of which is hereby incorporated by reference in its entirety.

Increase in ceramide level can have different outcomes leading to cell death and/or senescence. Ceramidase is the only enzyme that can hydrolyze ceramide and therefore, the only enzyme that can directly decrease the levels of ceramide in cells.

Table 2 contains the nucleotide sequences to be encoded by the vectors disclosed for use in practicing the methods described herein.

TABLE 2
Nucleotide Sequences Enzymes and Reporter Proteins

| Gene | Open Reading Frame |
|---|---|
| ASAH1 | ATGCCGGGCCGGAGTTGCGTCGCCTTAGTCCTCCTGGCTGCCGCCGTCA |
| transcript | GCTGTGCCGTCGCGCAGCACGCGCCGCCGTGGACAGAGGACTGCAGAA |
| variant 1 | AATCAACCTATCCTCCTTCAGGACCAACGTACAGAGGTGCAGTTCCATG |
| (ACv1) | GTACACCATAAATCTTGACTTACCACCCTACAAAAGATGGCATGAATTG |
| | ATGCTTGACAAGGCACCAGTGCTAAAGGTTATAGTGAATTCTCTGAAGA |
| | ATATGATAAATACATTCGTGCCAAGTGGAAAAATTATGCAGGTGGTGG |
| | ATGAAAAATTGCCTGGCCTACTTGGCAACTTTCCTGGCCCTTTTGAAGA |
| | GGAAATGAAGGGTATTGCCGCTGTTACTGATATACCTTTAGGAGAGATT |
| | ATTTCATTCAATATTTTTTATGAATTATTTACCATTTGTACTTCAATAGT |
| | AGCAGAAGACAAAAAAGGTCATCTAATACATGGGAGAAACATGGATTT |
| | TGGAGTATTTCTTGGGTGGAACATAAATAATGATACCTGGGTCATAACT |
| | GAGCAACTAAAACCTTTAACAGTGAATTTGGATTTCCAAAGAAACAAC |
| | AAAACTGTCTTCAAGGCTTCAAGCTTTGCTGGCTATGTGGGCATGTTAA |
| | CAGGATTCAAACCAGGACTGTTCAGTCTTACACTGAATGAACGTTTCAG |
| | TATAAATGGTGGTTATCTGGGTATTCTAGAATGGATTCTGGGAAAGAAA |
| | GATGTCATGTGGATAGGGTTCCTCACTAGAACAGTTCTGGAAAATAGCA |
| | CAAGTTATGAAGAAGCCAAGAATTTATTGACCAAGACCAAGATATTGG |
| | CCCCAGCCTACTTTATCCTGGGAGGCAACCAGTCTGGGGAAGGTTGTGT |
| | GATTACACGAGACAGAAAGGAATCATTGGATGTATATGAACTCGATGC |
| | TAAGCAGGGTAGATGGTATGTGGTACAAACAAATTATGACCGTTGGAA |
| | ACATCCCTTCTTCCTTGATGATCGCAGAACGCCTGCAAAGATGTGTCTG |
| | AACCGCACCAGCCAAGAGAATATCTCATTTGAAACCATGTATGATGTCC |
| | TGTCAACAAAACCTGTCCTCAACAAGCTGACCGTATACACAACCTTGAT |
| | AGATGTTACCAAAGGTCAATTCGAAACTTACCTGCGGGACTGCCCTGAC |
| | CCTTGTATAGGTTGGTGA (SEQ ID NO: 8) |
| Sphk1 | ATGGATCCAGTGGTCGGTTGCGGACGTGGCCTCTTTGGTTTTGTTTTCTC |
| | AGCGGGCGGCCCCCGGGGCGTGCTCCCGCGGCCCTGCCGCGTGCTGGT |
| | GCTGCTGAACCCGCGCGGCGGCAAGGGCAAGGCCTTGCAGCTCTTCCG |
| | GAGTCACGTGCAGCCCCTTTTGGCTGAGGCTGAAATCTCCTTCACGCTG |
| | ATGCTCACTGAGCGGCGGAACCACGCGCGGGAGCTGGTGCGGTCGGAG |
| | GAGCTGGGCCGCTGGGACGCTCTGGTGGTCATGTCTGGAGACGGGCTG |
| | ATGCACGAGGTGGTGAACGGGCTCATGGAGCGGCCTGACTGGGAGACC |
| | GCCATCCAGAAGCCCCTGTGTAGCCTCCCAGCAGGCTCTGGCAACGCGC |
| | TGGCAGCTTCCTTGAACCATTATGCTGGCTATGAGCAGGTCACCAATGA |
| | AGACCTCCTGACCAACTGCACGCTATTGCTGTGCCGCCGGCTGCTGTCA |
| | CCCATGAACCTGCTGTCTCTGCACACGGCTTCGGGGCTGCGCCTCTTCT |
| | CTGTGCTCAGCCTGGCCTGGGGCTTCATTGCTGATGTGGACCTAGAGAG |
| | TGAGAAGTATCGGCGTCTGGGGGAGATGCGCTTCACTCTGGGCACCTTC |
| | CTGCGTCTGGCAGCCCTGCGCACCTACCGCGGCCGACTGGCCTACCTCC |
| | CTGTAGGAAGAGTGGGTTCCAAGACACCTGCCTCCCCCGTTGTGGTCCA |
| | GCAGGGCCCGGTAGATGCACACCTTGTGCCACTGGAGGAGCCAGTGCC |
| | CTCTCACTGGACAGTGGTGCCCGACGAGGACTTTGTGCTAGTCCTGGCA |
| | CTGCTGCACTCGCACCTGGGCAGTGAGATGTTTGCTGCACCCATGGGCC |
| | GCTGTGCAGCTGGCGTCATGCATCTGTTCTACGTGCGGGGGGAGTGTC |
| | TCGTGCCATGCTGCTGCGCCTCTTCCTGGCCATGGAGAAGGGCAGGCAT |
| | ATGGAGTATGAATGCCCCTACTTGGTATATGTGCCCGTGGTCGCCTTCC |
| | GCTTGGAGCCCAAGGATGGGAAAGGTGTGTTTGCAGTGGATGGGGAAT |
| | TGATGGTTAGCGAGGCCGTGCAGGGCCAGGTGCACCCAAACTACTTCT |
| | GGATGGTCAGCGGTTGCGTGGAGCCCCCGCCCAGCTGGAAGCCCCAGC |
| | AGATGCCACCGCCAGAAGAGCCCTTATGA (SEQ ID NO: 9) |
| S1PR2 | ATGGGCAGCTTGTACTCGGAGTACCTGAACCCCAACAAGGTCCAGGAA |
| | CACTATAATTATACCAAGGAGACGCTGGAAACGCAGGAGACGACCTCC |
| | CGCCAGGTGGCCTCGGCCTTCATCGTCATCCTCTGTTGCGCCATTGTGGT |
| | GGAAAACCTTCTGGTGCTCATTGCGGTGGCCCGAAACAGCAAGTTCCAC |
| | TCGGCAATGTACCTGTTTCTGGGCAACCTGGCCGCCTCCGATCTACTGG |
| | CAGGCGTGGCCTTCGTAGCCAATACCTTGCTCTCTGGCTCTGTCACGCT |
| | GAGGCTGACGCCTGTGCAGTGGTTTGCCCGGGAGGGCTCTGCCTTCATC |
| | ACGCTCTCGGCCTCTGTCTTCAGCCTCCTGGCCATCGCCATTGAGCGCC |
| | ACGTGGCCATTGCCAAGGTCAAGCTGTATGGCAGCGACAAGAGCTGCC |
| | GCATGCTTCTGCTCATCGGGGCCTCGTGGCTCATCTCGCTGGTCCTCGGT |
| | GGCCTGCCCATCCTTGGCTGGAACTGCCTGGGCCACCTCGAGGCCTGCT |
| | CCACTGTCCTGCCTCTCTACGCCAAGCATTATGTGCTGTGCGTGGTGAC |
| | CATCTTCTCCATCATCCTGTTGGCCATCGTGGCCCTGTACGTGCGCATCT |
| | ACTGCGTGGTCCGCTCAAGCCACGCTGACATGGCCGCCCCGCAGACGCT |
| | AGCCCTGCTCAAGACGGTCACCATCGTGCTAGGCGTCTTTATCGTCTGC |
| | TGGCTGCCCGCCTTCAGCATCCTCCTTCTGGACTATGCCTGTCCCGTCCA |
| | CTCCTGCCCGATCCTCTACAAAGCCCACTACTTTTTCGCCGTCTCCACCC |
| | TGAATTCCCTGCTCAACCCCGTCATCTACACGTGGCGCAGCCGGGACCT |
| | GCGGCGGGAGGTGCTTCGGCCGCTGCAGTGCTGGAGGCCGGGGGTGGG |
| | GGTGCAAGGACGGAGGCGGGGGGGGACCCCGGGCCACCACCTCCTGCC |
| | ACTCCGCAGCTCCAGCTCCCTGGAGAGGGGCATGCACATGCCCACGTC |
| | ACCCACGTTTCTGGAGGGCAACACGGTGGTCATG (SEQ ID NO: 10) |
| Firefly | ATGGCCGATGCTAAGAACATTAAGAAGGGCCCTGCTCCCTTCTACCCTC |
| luciferase | TGGAGGATGGCACCGCTGGCGAGCAGCTGCACAAGGCCATGAAGAGGT |
| | ATGCCCTGGTGCCTGGCACCATTGCCTTCACCGATGCCCACATTGAGGT |
| | GGACATCACCTATGCCGAGTACTTCGAGATGTCTGTGCGCCTGGCCGAG |
| | GCCATGAAGAGGTACGGCCTGAACACCAACCACCGCATCGTGGTGTGC |
| | TCTGAGAACTCTCTGCAGTTCTTCATGCCAGTGCTGGGCGCCCTGTTCAT |
| | CGGAGTGGCCGTGGCCCCTGCTAACGACATTTACAACGAGCGCGAGCT |
| | GCTGAACAGCATGGGCATTTCTCAGCCTACCGTGGTGTTCGTGTCTAAG |
| | AAGGGCCTGCAGAAGATCCTGAACGTGCAGAAGAAGCTGCCTATCATC |
| | CAGAAGATCATCATCATGGACTCTAAGACCGACTACCAGGGCTTCCAG |
| | AGCATGTACACATTCGTGACATCTCATCTGCCTCCTGGCTTCAACGAGT |
| | ACGACTTCGTGCCAGAGTCTTTCGACAGGGACAAAACCATTGCCCTGAT |
| | CATGAACAGCTCTGGGTCTACCGGCCTGCCTAAGGGCGTGGCCCTGCCT |
| | CATCGCACCGCCTGTGTGCGCTTCTCTCACGCCCGCGACCCTATTTTCGG |
| | CAACCAGATCATCCCCGACACCGCTATTCTGAGCGTGGTGCCATTCCAC |
| | CACGGCTTCGGCATGTTCACCACCCTGGGCTACCTGATTTGCGGCTTTC |
| | GGGTGGTGCTGATGTACCGCTTCGAGGAGGAGCTGTTCCTGCGCAGCCT |
| | GCAAGACTACAAAATTCAGTCTGCCCTGCTGGTGCCAACCCTGTTCAGC |
| | TTCTTCGCTAAGAGCACCCTGATCGACAAGTACGACCTGTCTAACCTGC |
| | ACGAGATTGCCTCTGGCGGCGCCCCACTGTCTAAGGAGGTGGGCGAAG |
| | CCGTGGCCAAGCGCTTTCATCTGCCAGGCATCCGCCAGGGCTACGGCCT |
| | GACCGAGACAACCAGCGCCATTCTGATTACCCCAGAGGGCGACGACAA |
| | GCCTGGCGCCGTGGGCAAGGTGGTGCCATTCTTCGAGGCCAAGGTGGT |
| | GGACCTGGACACCGGCAAGACCCTGGGAGTGAACCAGCGCGGCGAGCT |
| | GTGTGTGCGCGGCCCTATGATTATGTCCGGCTACGTGAATAACCCTGAG |
| | GCCACAAACGCCCTGATCGACAAGGACGGCTGGCTGCACTCTGGCGAC |
| | ATTGCCTACTGGGACGAGGACGAGCACTTCTTCATCGTGGACCGCCTGA |
| | AGTCTCTGATCAAGTACAAGGGCTACCAGGTGGCCCCAGCCGAGCTGG |
| | AGTCTATCCTGCTGCAGCACCCTAACATTTTCGACGCCGGAGTGGCCGG |
| | CCTGCCCGACGACGATGCCGGCGAGCTGCCTGCCGCCGTCGTCGTGCTG |
| | GAACACGGCAAGACCATGACCGAGAAGGAGATCGTGGACTATGTGGCC |
| | AGCCAGGTGACAACCGCCAAGAAGCTGCGCGGCGGAGTGGTGTTCGTG |
| | GACGAGGTGCCCAAGGGCCTGACCGGCAAGCTGGACGCCCGCAAGATC |
| | CGCGAGATCCTGATCAAGGCTAAGAAAGGCGGCAAGATCGCCGTGTAA |
| | (SEQ ID NO: 11) |
| nGFP | ATGGTGAGCAAGGGCGAGGAGCTGTTCACCGGGGTGGTGCCCATCCTG |
| | GTCGAGCTGGACGGCGACGTAAACGGCCACAAGTTCAGCGTGTCCGGC |
| | GAGGGCGAGGGCGATGCCACCTACGGCAAGCTGACCCTGAAGTTCATC |
| | TGCACCACCGGCAAGCTGCCCGTGCCCTGGCCCACCCTCGTGACCACCC |
| | TGACCTACGGCGTGCAGTGCTTCAGCCGCTACCCCGACCACATGAAGCA |
| | GCACGACTTCTTCAAGTCCGCCATGCCCGAAGGCTACGTCCAGGAGCGC |
| | ACCATCTTCTTCAAGGACGACGGCAACTACAAGACCCGCGCCGAGGTG |
| | AAGTTCGAGGGCGACACCCTGGTGAACCGCATCGAGCTGAAGGGCATC |
| | GACTTCAAGGAGGACGGCAACATCCTGGGGCACAAGCTGGAGTACAAC |
| | TACAACAGCCACAACGTCTATATCATGGCCGACAAGCAGAAGAACGGC |
| | ATCAAGGTGAACTTCAAGATCCGCCACAACATCGAGGACGGCAGCGTG |
| | CAGCTCGCCGACCACTACCAGCAGAACACCCCCATCGGCGACGGCCCC |
| | GTGCTGCTGCCCGACAACCACTACCTGAGCACCCAGTCCGCCCTGAGCA |
| | AAGACCCCAACGAGAAGCGCGATCACATGGTCCTGCTGGAGTTCGTGA |
| | CCGCCGCCGGGATCACTCTCGGCATGGACGAGCTGTACAAGGGAGATC |
| | CAAAAAAGAAGAGAAAGGTAGGCGATCCAAAAAAGAAGAGAAAGGTA |
| | GGTGATCCAAAAAAGAAGAGAAAGGTATAA (SEQ ID NO: 12) |
| ASAH1 | ATGAACTGCTGCATCGGGCTGGGAGAGAAAGCTCGCGGGTCCCACCGG |
| transcript | GCCTCCTACCCAAGTCTCAGCGCGCTTTTCACCGAGGCCTCAATTCTGG |
| variant 2 | GATTTGGCAGCTTTGCTGTGAAAGCCCAATGGACAGAGGACTGCAGAA |
| (ACv2) | AATCAACCTATCCTCCTTCAGGACCAACGTACAGAGGTGCAGTTCCATG |
| | GTACACCATAAATCTTGACTTACCACCCTACAAAAGATGGCATGAATTG |
| | ATGCTTGACAAGGCACCAGTGCTAAAGGTTATAGTGAATTCTCTGAAGA |
| | ATATGATAAATACATTCGTGCCAAGTGGAAAAATTATGCAGGTGGTGG |
| | ATGAAAAATTGCCTGGCCTACTTGGCAACTTTCCTGGCCCTTTTGAAGA |
| | GGAAATGAAGGGTATTGCCGCTGTTACTGATATACCTTTAGGAGAGATT |
| | ATTTCATTCAATATTTTTTATGAATTATTTACCATTTGTACTTCAATAGT |
| | AGCAGAAGACAAAAAAGGTCATCTAATACATGGGAGAAACATGGATTT |
| | TGGAGTATTTCTTGGGTGGAACATAAATAATGATACCTGGGTCATAACT |
| | GAGCAACTAAAACCTTTAACAGTGAATTTGGATTTCCAAAGAAACAAC |
| | AAAACTGTCTTCAAGGCTTCAAGCTTTGCTGGCTATGTGGGCATGTTAA |
| | CAGGATTCAAACCAGGACTGTTCAGTCTTACACTGAATGAACGTTTCAG |
| | TATAAATGGTGGTTATCTGGGTATTCTAGAATGGATTCTGGGAAAGAAA |
| | GATGTCATGTGGATAGGGTTCCTCACTAGAACAGTTCTGGAAAATAGCA |
| | CAAGTTATGAAGAAGCCAAGAATTTATTGACCAAGACCAAGATATTGG |
| | CCCCAGCCTACTTTATCCTGGGAGGCAACCAGTCTGGGGAAGGTTGTGT |
| | GATTACACGAGACAGAAAGGAATCATTGGATGTATATGAACTCGATGC |
| | TAAGCAGGGTAGATGGTATGTGGTACAAACAAATTATGACCGTTGGAA |
| | ACATCCCTTCTTCCTTGATGATCGCAGAACGCCTGCAAAGATGTGTCTG |
| | AACCGCACCAGCCAAGAGAATATCTCATTTGAAACCATGTATGATGTCC |
| | TGTCAACAAAACCTGTCCTCAACAAGCTGACCGTATACACAACCTTGAT |
| | AGATGTTACCAAAGGTCAATTCGAAACTTACCTGCGGGACTGCCCTGAC |
| | CCTTGTATAGGTTGGTGA (SEQ ID NO: 13) |
| ASAH1 | ATGAACTGCTGCATCGGGCTGGGAGAGAAAGCTCGCGGGTCCCACCGG |
| transcript | GCCTCCTACCCAAGTCTCAGCGCGCTTTTCACCGAGGCCTCAATTCTGG |
| variant 3 | GATTTGGCAGCTTTGCTGTGAAAGCCCAATGGACAGAGGACTGCAGAA |
| | AATCAACCTATCCTCCTTCAGGACCAACTGTCTTCCCTGCTGTTATAAG |
| | GTACAGAGGTGCAGTTCCATGGTACACCATAAATCTTGACTTACCACCC |
| | TACAAAAGATGGCATGAATTGATGCTTGACAAGGCACCAGTGCCTGGC |
| | CTACTTGGCAACTTTCCTGGCCCTTTTGAAGAGGAAATGAAGGGTATTG |
| | CCGCTGTTACTGATATACCTTTAGGAGAGATTATTTCATTCAATATTTTT |
| | TATGAATTATTTACCATTTGTACTTCAATAGTAGCAGAAGACAAAAAAG |
| | GTCATCTAATACATGGGAGAAACATGGATTTTGGAGTATTTCTTGGGTG |
| | GAACATAAATAATGATACCTGGGTCATAACTGAGCAACTAAAACCTTT |
| | AACAGTGAATTTGGATTTCCAAAGAAACAACAAAACTGTCTTCAAGGC |
| | TTCAAGCTTTGCTGGCTATGTGGGCATGTTAACAGGATTCAAACCAGGA |
| | CTGTTCAGTCTTACACTGAATGAACGTTTCAGTATAAATGGTGGTTATC |
| | TGGGTATTCTAGAATGGATTCTGGGAAAGAAAGATGTCATGTGGATAG |
| | GGTTCCTCACTAGAACAGTTCTGGAAAATAGCACAAGTTATGAAGAAG |
| | CCAAGAATTTATTGACCAAGACCAAGATATTGGCCCCAGCCTACTTTAT |
| | CCTGGGAGGCAACCAGTCTGGGGAAGGTTGTGTGATTACACGAGACAG |
| | AAAGGAATCATTGGATGTATATGAACTCGATGCTAAGCAGGGTAGATG |
| | GTATGTGGTACAAACAAATTATGACCGTTGGAAACATCCCTTCTTCCTT |
| | GATGATCGCAGAACGCCTGCAAAGATGTGTCTGAACCGCACCAGCCAA |
| | GAGAATATCTCATTTGAAACCATGTATGATGTCCTGTCAACAAAACCTG |
| | TCCTCAACAAGCTGACCGTATACACAACCTTGATAGATGTTACCAAAGG |
| | TCAATTCGAAACTTACCTGCGGGACTGCCCTGACCCTTGTATAGGTTGG |
| | TGA (SEQ ID NO: 14) |
| ASAH2 | ATGGCCAAACGCACCTTCTCTAACTTGGAGACATTCCTGATTTTCCTCCT |
| transcript | TGTAATGATGAGTGCCATCACAGTGGCCCTTCTCAGCCTCTTGTTTATCA |
| variant 1 | CCAGTGGGACCATTGAAAACCACAAAGATTTAGGAGGCCATTTTTTTTC |
| | AACCACCCAAAGCCCTCCAGCCACCCAGGGCTCCACAGCTGCCCAACG |
| | CTCCACAGCCACCCAGCATTCCACAGCCACCCAGAGCTCCACAGCCACT |
| | CAAACTTCTCCAGTGCCTTTAACCCCAGAGTCTCCTCTATTTCAGAACTT |
| | CAGTGGCTACCATATTGGTGTTGGACGAGCTGACTGCACAGGACAAGT |
| | AGCAGATATCAATTTGATGGGCTATGGCAAATCCGGCCAGAATGCACA |
| | GGGCATCCTCACCAGGCTATACAGTCGTGCCTTCATCATGGCAGAACCT |
| | GATGGGTCCAATCGAACAGTGTTTGTCAGCATCGACATAGGCATGGTAT |
| | CACAAAGGCTCAGGCTGGAGGTCCTGAACAGACTGCAGAGTAAATATG |
| | GCTCCCTGTACAGAAGAGATAATGTCATCCTGAGTGGCACTCACACTCA |
| | TTCAGGTCCTGCAGGATATTTCCAGTATACCGTGTTTGTAATTGCCAGT |
| | GAAGGATTTAGCAATCAAACTTTTCAGCACATGGTCACTGGTATCTTGA |
| | AGAGCATTGACATAGCACACACAAATATGAAACCAGGCAAAATCTTCA |
| | TCAATAAAGGAAATGTGGATGGTGTGCAGATCAACAGAAGTCCGTATT |
| | CTTACCTTCAAAATCCGCAGTCAGAGAGAGCAAGGTATTCTTCAAATAC |
| | AGACAAGGAAATGATAGTTTTGAAAATGGTAGATTTGAATGGAGATGA |
| | CTTGGGCCTTATCAGCTGGTTTGCCATCCACCCGGTCAGCATGAACAAC |
| | AGTAACCATCTTGTAAACAGTGACAATGTGGGCTATGCATCTTACCTGC |
| | TTGAGCAAGAGAAGAACAAAGGATATCTACCTGGACAGGGGCCATTTG |
| | TAGCAGCCTTTGCTTCATCAAACCTAGGAGATGTGTCCCCCAACATTCT |
| | TGGACCACGTTGCATCAACACAGGAGAGTCCTGTGATAACGCCAATAG |
| | CACTTGTCCCATTGGTGGGCCTAGCATGTGCATTGCTAAGGGACCTGGA |
| | CAGGATATGTTTGACAGCACACAAATTATAGGACGGGCCATGTATCAG |
| | AGAGCAAAGGAACTCTATGCCTCTGCCTCCCAGGAGGTAACAGGACCA |
| | CTGGCTTCAGCACACCAGTGGGTGGATATGACAGATGTGACTGTCTGGC |
| | TCAATTCCACACATGCATCAAAAACATGTAAACCAGCATTGGGCTACA |
| | GTTTTGCAGCTGGCACTATTGATGGAGTTGGAGGCCTCAATTTTACACA |
| | GGGGAAAACAGAAGGGGATCCATTTTGGGACACCATTCGGGACCAGAT |
| | CCTGGGAAAGCCATCTGAAGAAATTAAAGAATGTCATAAACCAAAGCC |
| | CATCCTTCTTCACACCGGAGAACTATCAAAACCTCACCCCTGGCATCCA |
| | GACATTGTTGATGTTCAGATTATTACCCTTGGGTCCTTGGCCATAACTGC |
| | CATCCCCGGGGAGTTTACGACCATGTCTGGACGAAGACTTCGAGAGGC |
| | AGTTCAAGCAGAATTTGCATCTCATGGGATGCAGAACATGACTGTTGTT |
| | ATTTCAGGTCTATGCAACGTCTATACACATTACATTACCACTTATGAAG |
| | AATACCAGGCTCAGCGATATGAGGCAGCATCGACAATTTATGGACCGC |
| | ACACATTATCTGCTTACATTCAGCTCTTCAGAAACCTTGCTAAGGCTATT |
| | GCTACGGACACGGTAGCCAACCTGAGCAGAGGTCCAGAACCTCCCTTTT |
| | TCAAACAATTAATAGTTCCATTAATTCCTAGTATTGTGGATAGAGCACC |
| | AAAAGGCAGAACTTTCGGGGATGTCCTGCAGCCAGCAAAACCTGAATA |
| | CAGAGTGGGGGAAGTTGCTGAAGTTATATTTGTAGGTGCTAACCCGAA |
| | GAATTCAGTACAAAACCAGACCCATCAGACCTTCCTCACTGTGGAGAA |
| | ATATGAGGCTACTTCAACATCGTGGCAGATAGTGTGTAATGATGCCTCC |
| | TGGGAGACTCGTTTTTATTGGCACAAGGGACTCCTGGGTCTGAGTAATG |
| | CAACAGTGGAATGGCATATTCCAGACACTGCCCAGCCTGGAATCTACA |
| | GAATAAGATATTTTGGACACAATCGGAAGCAGGACATTCTGAAGCCTG |
| | CTGTCATACTTTCATTTGAAGGCACTTCCCCGGCTTTTGAAGTTGTAACT |
| | ATTTAGTGA (SEQ ID NO: 15) |
| ASAH2 | ATGGCCAAACGCACCTTCTCTAACTTGGAGACATTCCTGATTTTCCTCCT |
| transcript | TGTAATGATGAGTGCCATCACAGTGGCCCTTCTCAGCCTCTTGTTTATCA |
| variant 2 | CCAGTGGGACCATTGAAAACCACAAAGATTTAGGAGGCCATTTTTTTTC |
| | AACCACCCAAAGCCCTCCAGCCACCCAGGGCTCCACAGCTGCCCAACG |
| | CTCCACAGCCACCCAGCATTCCACAGCCACCCAGAGCTCCACAGCCACT |
| | CAAACTTCTCCAGTGCCTTTAACCCCAGAGTCTCCTCTATTTCAGAACTT |
| | CAGTGGCTACCATATTGGTGTTGGACGAGCTGACTGCACAGGACAAGT |
| | AGCAGATATCAATTTGATGGGCTATGGCAAATCCGGCCAGAATGCACA |
| | GGGCATCCTCACCAGGCTATACAGTCGTGCCTTCATCATGGCAGAACCT |
| | GATGGGTCCAATCGAACAGTGTTTGTCAGCATCGACATAGGCATGGTAT |
| | CACAAAGGCTCAGGCTGGAGGTCCTGAACAGACTGCAGAGTAAATATG |
| | GCTCCCTGTACAGAAGAGATAATGTCATCCTGAGTGGCACTCACACTCA |
| | TTCAGGTCCTGCAGGATATTTCCAGTATACCGTGTTTGTAATTGCCAGT |
| | GAAGGATTTAGCAATCAAACTTTTCAGCACATGGTCACTGGTATCTTGA |
| | AGAGCATTGACATAGCACACACAAATATGAAACCAGGCAAAATCTTCA |
| | TCAATAAAGGAAATGTGGATGGTGTGCAGATCAACAGAAGTCCGTATT |
| | CTTACCTTCAAAATCCGCAGTCAGAGAGAGCAAGGTATTCTTCAAATAC |
| | AGACAAGGAAATGATAGTTTTGAAAATGGTAGATTTGAATGGAGATGA |
| | CTTGGGCCTTATCAGCTGGTTTGCCATCCACCCGGTCAGCATGAACAAC |
| | AGTAACCATCTTGTAAACAGTGACAATGTGGGCTATGCATCTTACCTGC |
| | TTGAGCAAGAGAAGAACAAAGGATATCTACCTGGACAGGGGCCATTTG |
| | TAGCAGCCTTTGCTTCATCAAACCTAGGAGATGTGTCCCCCAACATTCT |
| | TGGACCACGTTGCATCAACACAGGAGAGTCCTGTGATAACGCCAATAG |
| | CACTTGTCCCATTGGTGGGCCTAGCATGTGCATTGCTAAGGGACCTGGA |
| | CAGGATATGTTTGACAGCACACAAATTATAGGACGGGCCATGTATCAG |
| | AGAGCAAAGTCAAAAACATGTAAACCAGCATTGGGCTACAGTTTTGCA |
| | GCTGGCACTATTGATGGAGTTGGAGGCCTCAATTTTACACAGGGGAAA |
| | ACAGAAGGGGATCCATTTTGGGACACCATTCGGGACCAGATCCTGGGA |
| | AAGCCATCTGAAGAAATTAAAGAATGTCATAAACCAAAGCCCATCCTT |
| | CTTCACACCGGAGAACTATCAAAACCTCACCCCTGGCATCCAGACATTG |
| | TTGATGTTCAGATTATTACCCTTGGGTCCTTGGCCATAACTGCCATCCCC |
| | GGGGAGTTTACGACCATGTCTGGACGAAGACTTCGAGAGGCAGTTCAA |
| | GCAGAATTTGCATCTCATGGGATGCAGAACATGACTGTTGTTATTTCAG |
| | GTCTATGCAACGTCTATACACATTACATTACCACTTATGAAGAATACCA |
| | GGCTCAGCGATATGAGGCAGCATCGACAATTTATGGACCGCACACATT |
| | ATCTGCTTACATTCAGCTCTTCAGAAACCTTGCTAAGGCTATTGCTACG |
| | GACACGGTAGCCAACCTGAGCAGAGGTCCAGAACCTCCCTTTTTCAAAC |
| | AATTAATAGTTCCATTAATTCCTAGTATTGTGGATAGAGCACCAAAAGG |
| | CAGAACTTTCGGGGATGTCCTGCAGCCAGCAAAACCTGAATACAGAGT |
| | GGGGGAAGTTGCTGAAGTTATATTTGTAGGTGCTAACCCGAAGAATTCA |
| | GTACAAAACCAGACCCATCAGACCTTCCTCACTGTGGAGAAATATGAG |
| | GCTACTTCAACATCGTGGCAGATAGTGTGTAATGATGCCTCCTGGGAGA |
| | CTCGTTTTTATTGGCACAAGGGACTCCTGGGTCTGAGTAATGCAACAGT |
| | GGAATGGCATATTCCAGACACTGCCCAGCCTGGAATCTACAGAATAAG |
| | ATATTTTGGACACAATCGGAAGCAGGACATTCTGAAGCCTGCTGTCATA |
| | CTTTCATTTGAAGGCACTTCCCCGGCTTTTGAAGTTGTAACTATTTAGTG |
| | A (SEQ ID NO: 16) |
| ASAH2B | ATGAGGCAGCATCGACAATTTATGGACCGCACGCATTATCTGCTTACAT |
| transcript | TCAGCTCTTCAGAAACCTTGCTAAGGCTATTGCTACGTATTGTGGATAG |
| variant 1 | AGCACCAAAAGGCAGAACTTTCGGGGATGTCCTGCAGCCAGCAAAACC |
| | TGAATACAGAGTGGGGGAAGTTGCTGAAGTTATATTTGTAGGTGCTAAC |
| | CCGAAGAATTCAGTACAAAACCAGACCCATCAGACCTTCCTCACTGTGG |
| | AGAAATATGAGGCTACTTCAACATCGTGGCAGATAGTGTGTAATGATG |
| | CCTCCTGGGAGACTCGTTTTTATTGGCACAAGGGACTCCTGGGTCTGAG |
| | TAATGCAACAGTGGAATGGCATATTCCAGACACTGCCCAGCCTGGAAT |
| | CTACAGAATAAGATATTTTGGACACAATCGGAAGCAGGACATTCTGAA |
| | GCCTGCTGTCATACTTTCATTTGAAGGCACTTCCCCGGCTTTTGAAGTTG |
| | TAACTATTTAGTGA (SEQ ID NO: 17) |
| ASAH2B | ATGGTAGCCAACCTGAGCAGAGGTCCAGAACCTCCCTTTTTCAAACAAT |
| transcript | TAATAGTTCCATTAATTCCTAGTATTGTGGATAGAGCACCAAAAGGCAG |
| variant 3 | AACTTTCGGGGATGTCCTGCAGCCAGCAAAACCTGAATACAGAGTGGG |
| | GGAAGTTGCTGAAGTTATATTTGTAGGTGCTAACCCGAAGAATTCAGTA |
| | CAAAACCAGACCCATCAGACCTTCCTCACTGTGGAGAAATATGAGGCT |
| | ACTTCAACATCGTGGCAGATAGTGTGTAATGATGCCTCCTGGGAGACTC |
| | GTTTTTATTGGCACAAGGGACTCCTGGGTCTGAGTAATGCAACAGTGGA |
| | ATGGCATATTCCAGACACTGCCCAGCCTGGAATCTACAGAATAAGATAT |
| | TTTGGACACAATCGGAAGCAGGACATTCTGAAGCCTGCTGTCATACTTT |
| | CATTTGAAGGCACTTCCCCGGCTTTTGAAGTTGTAACTATTTAGTGAAT |
| | GGTAGCCAACCTGAGCAGAGGTCCAGAACCTCCCTTTTTCAAACAATTA |
| | ATAGTTCCATTAATTCCTAGTATTGTGGATAGAGCACCAAAAGGCAGAA |
| | CTTTCGGGGATGTCCTGCAGCCAGCAAAACCTGAATACAGAGTGGGGG |
| | AAGTTGCTGAAGTTATATTTGTAGGTGCTAACCCGAAGAATTCAGTACA |
| | AAACCAGACCCATCAGACCTTCCTCACTGTGGAGAAATATGAGGCTACT |
| | TCAACATCGTGGCAGATAGTGTGTAATGATGCCTCCTGGGAGACTCGTT |
| | TTTATTGGCACAAGGGACTCCTGGGTCTGAGTAATGCAACAGTGGAATG |
| | GCATATTCCAGACACTGCCCAGCCTGGAATCTACAGAATAAGATATTTT |
| | GGACACAATCGGAAGCAGGACATTCTGAAGCCTGCTGTCATACTTTCAT |
| | TTGAAGGCACTTCCCCGGCTTTTGAAGTTGTAACTATTTAGTGA (SEQ ID |
| | NO: 18) |
| ASAH2B | ATGGTAGCCAACCTGAGCAGAGGTCCAGAACCTCCCTTTTTCAAACAAT |
| transcript | TAATAGTTCCATTAATTCCTAGTATTGTGGATAGAGCACCAAAAGGCAG |
| variant 4 | AACTTTCGGGGATGTCCTGCAGCCAGCAAAACCTGAATACAGAGTGGG |
| | GGAAGTTGCTGAAGTTATATTTGTAGGTGCTAACCCGAAGAATTCAGTA |
| | CAAAACCAGACCCATCAGACCTTCCTCACTGTGGAGAAATATGAGGCT |
| | ACTTCAACATCGTGGCAGATAGTGTGTAATGATGCCTCCTGGGAGACTC |
| | GTTTTTATTGGCACAAGGGACTCCTGGGTCTGAGTAATGCAACAGTGGA |
| | ATGGCATATTCCAGACACTGCCCAGCCTGGAATCTACAGAATAAGATAT |
| | TTTGGACACAATCGGAAGCAGGACATTCTGAAGCCTGCTGTCATACTTT |
| | CATTTGAAGGCACTTCCCCGGCTTTTGAAGTTGTAACTATTTAG (SEQ ID |
| | NO: 19) |
| ACER1 | ATGCCTAGCATCTTCGCCTATCAGAGCTCCGAGGTGGACTGGTGTGAGA |
| | GCAACTTCCAGTACTCGGAGCTGGTGGCCGAGTTCTACAACACGTTCTC |
| | CAATATCCCCTTCTTCATCTTCGGGCCACTGATGATGCTCCTGATGCACC |
| | CGTATGCCCAGAAGCGCTCCCGCTACATTTACGTTGTCTGGGTCCTCTTC |
| | ATGATCATAGGCCTGTTCTCCATGTATTTCCACATGACGCTCAGCTTCCT |
| | GGGCCAGCTGCTGGACGAGATCGCCATCCTGTGGCTCCTGGGCAGTGG |
| | CTATAGCATATGGATGCCCCGCTGCTATTTCCCCTCCTTCCTTGGGGGG |
| | AACAGGTCCCAGTTCATCCGCCTGGTCTTCATCACCACTGTGGTCAGCA |
| | CCCTTCTGTCCTTCCTGCGGCCCACGGTCAACGCCTACGCCCTCAACAG |
| | CATTGCCCTGCACATTCTCTACATCGTGTGCCAGGAGTACAGGAAGACC |
| | AGCAATAAGGAGCTTCGGCACCTGATTGAGGTCTCCGTGGTTTTATGGG |
| | CTGTTGCTCTGACCAGCTGGATCAGTGACCGTCTGCTTTGCAGCTTCTG |
| | GCAGAGGATTCATTTCTTCTATCTGCACAGCATCTGGCATGTGCTCATC |
| | AGCATCACCTTCCCTTATGGCATGGTCACCATGGCCTTGGTGGATGCCA |
| | ACTATGAGATGCCAGGTGAAACCCTCAAAGTCCGCTACTGGCCTCGGG |
| | ACAGTTGGCCCGTGGGGCTGCCCTACGTGGAAATCCGGGGTGATGACA |
| | AGGACTGCTGA (SEQ ID NO: 20) |
| ACER2 | ATGGGCGCCCCGCACTGGTGGGACCAGCTGCAGGCTGGTAGCTCGGAG |
| | GTGGACTGGTGCGAGGACAACTACACCATCGTGCCTGCTATCGCCGAGT |
| | TCTACAACACGATCAGCAATGTCTTATTTTTCATTTTACCGCCCATCTGC |
| | ATGTGCTTGTTTCGTCAGTATGCAACATGCTTCAACAGTGGCATCTACTT |
| | AATCTGGACTCTTTTGGTTGTAGTGGGAATTGGATCCGTCTACTTCCATG |
| | CAACCCTTAGTTTCTTGGGTCAGATGCTTGATGAACTTGCAGTCCTTTGG |
| | GTTCTGATGTGTGCTTTGGCCATGTGGTTCCCCAGAAGGTATCTACCAA |
| | AGATCTTTCGGAATGACCGGGGTAGGTTCAAGGTGGTGGTCAGTGTCCT |
| | GTCTGCGGTTACGACGTGCCTGGCATTTGTCAAGCCTGCCATCAACAAC |
| | ATCTCTCTGATGACCCTGGGAGTTCCTTGCACTGCACTGCTCATCGCAG |
| | AGCTAAAGAGGTGTGACAACATGCGTGTGTTTAAGCTGGGCCTCTTCTC |
| | GGGCCTCTGGTGGACCCTGGCCCTGTTCTGCTGGATCAGTGACCGAGCT |
| | TTCTGCGAGCTGCTGTCATCCTTCAACTTCCCCTACCTGCACTGCATGTG |
| | GCACATCCTCATCTGCCTTGCTGCCTACCTGGGCTGTGTATGCTTTGCCT |
| | ACTTTGATGCTGCCTCAGAGATTCCTGAGCAAGGCCCTGTCATCAAGTT |
| | CTGGCCCAATGAGAAATGGGCCTTCATTGGTGTCCCCTATGTGTCCCTC |
| | CTGTGTGCCAACAAGAAATCATCAGTCAAGATCACGTGA (SEQ ID NO: |
| | 21) |
| ACER3 | ATGGCTCCGGCCGCGGACCGAGAGGGCTACTGGGGCCCCACGACCTCC |
| transcript | ACGCTGGACTGGTGCGAGGAGAACTACTCCGTGACCTGGTACATCGCC |
| variant 1 | GAGTTCTGGAATACAGTGAGTAACCTGATCATGATTATACCTCCAATGT |
| | TCGGTGCAGTTCAGAGTGTTAGAGACGGTCTGGAAAAGCGGTACATTG |
| | CTTCTTATTTAGCACTCACAGTGGTAGGAATGGGATCCTGGTGCTTCCA |
| | CATGACTCTGAAATATGAAATGCAGCTATTGGATGAACTCCCAATGATA |
| | TACAGCTGTTGCATATTTGTGTACTGCATGTTTGAATGTTTCAAGATCAA |
| | GAACTCAGTAAACTACCATCTGCTTTTTACCTTAGTTCTATTCAGTTTAA |
| | TAGTAACCACAGTTTACCTTAAGGTAAAAGAGCCGATATTCCATCAGGT |
| | CATGTATGGAATGTTGGTCTTTACATTAGTACTTCGATCTATTTATATTG |
| | TTACATGGGTTTATCCATGGCTTAGAGGACTGGGTTATACATCATTGGG |
| | TATATTTTTATTGGGATTTTTATTTTGGAATATAGATAACATATTTTGTG |
| | AGTCACTGAGGAACTTTCGAAAGAAGGTACCACCTATCATAGGTATTAC |
| | CACACAATTTCATGCATGGTGGCATATTTTAACTGGCCTTGGTTCCTATC |
| | TTCACATCCTTTTCAGTTTGTATACAAGAACACTTTACCTGAGATATAG |
| | GCCAAAAGTGAAGTTTCTCTTTGGAATCTGGCCAGTGATCCTGTTTGAG |
| | CCTCTCAGGAAGCATTGA (SEQ ID NO: 22) |
| ACER3 | ATGGCTCCGGCCGCGGACCGAGAGGGCTACTGGGGCCCCACGACCTCC |
| transcript | ACGCTGGACTGGTGCGAGGAGAACTACTCCGTGACCTGGTACATCGCC |
| variant 2 | GAGTTCTTGGTAGGAATGGGATCCTGGTGCTTCCACATGACTCTGAAAT |
| | ATGAAATGCAGCTATTGGATGAACTCCCAATGATATACAGCTGTTGCAT |
| | ATTTGTGTACTGCATGTTTGAATGTTTCAAGATCAAGAACTCAGTAAAC |
| | TACCATCTGCTTTTTACCTTAGTTCTATTCAGTTTAATAGTAACCACAGT |
| | TTACCTTAAGGTAAAAGAGCCGATATTCCATCAGGTCATGTATGGAATG |
| | TTGGTCTTTACATTAGTACTTCGATCTATTTATATTGTTACATGGGTTTA |
| | TCCATGGCTTAGAGGACTGGGTTATACATCATTGGGTATATTTTTATTG |
| | GGATTTTTATTTTGGAATATAGATAACATATTTTGTGAGTCACTGAGGA |
| | ACTTTCGAAAGAAGGTACCACCTATCATAGGTATTACCACACAATTTCA |
| | TGCATGGTGGCATATTTTAACTGGCCTTGGTTCCTATCTTCACATCCTTT |
| | TCAGTTTGTATACAAGAACACTTTACCTGAGATATAGGCCAAAAGTGAA |
| | GTTTCTCTTTGGAATCTGGCCAGTGATCCTGTTTGAGCCTCTCAGGAAG |
| | CATTGA (SEQ ID NO: 23) |
| ACER3 | ATGATATACAGCTGTTGCATATTTGTGTACTGCATGTTTGAATGTTTCAA |
| transcript | GATCAAGAACTCAGTAAACTACCATCTGCTTTTTACCTTAGTTCTATTCA |
| variant 3 | GTTTAATAGTAACCACAGTTTACCTTAAGGTAAAAGAGCCGATATTCCA |
| | TCAGGTCATGTATGGAATGTTGGTCTTTACATTAGTACTTCGATCTATTT |
| | ATATTGTTACATGGGTTTATCCATGGCTTAGAGGACTGGGTTATACATC |
| | ATTGGGTATATTTTTATTGGGATTTTTATTTTGGAATATAGATAACATAT |
| | TTTGTGAGTCACTGAGGAACTTTCGAAAGAAGGTACCACCTATCATAGG |
| | TATTACCACACAATTTCATGCATGGTGGCATATTTTAACTGGCCTTGGTT |
| | CCTATCTTCACATCCTTTTCAGTTTGTATACAAGAACACTTTACCTGAGA |
| | TATAGGCCAAAAGTGAAGTTTCTCTTTGGAATCTGGCCAGTGATCCTGT |
| | TTGAGCCTCTCAGGAAGCATTGA (SEQ ID NO: 24) |
| Sphk2 | ATGAATGGACACCTTGAAGCAGAGGAGCAGCAGGACCAGAGGCCAGA |
| | CCAGGAGCTGACCGGGAGCTGGGGCCACGGGCCTAGGAGCACCCTGGT |
| | CAGGGCTAAGGCCATGGCCCCGCCCCCACCGCCACTGGCTGCCAGCAC |
| | CCCGCTCCTCCATGGCGAGTTTGGCTCCTACCCAGCCCGAGGCCCACGC |
| | TTTGCCCTCACCCTTACATCGCAGGCCCTGCACATACAGCGGCTGCGCC |
| | CCAAACCTGAAGCCAGGCCCCGGGGTGGCCTGGTCCCGTTGGCCGAGG |
| | TCTCAGGCTGCTGCACCCTGCGAAGCCGCAGCCCCTCAGACTCAGCGGC |
| | CTACTTCTGCATCTACACCTACCCTCGGGGCCGGCGCGGGGCCCGGCGC |
| | AGAGCCACTCGCACCTTCCGGGCAGATGGGGCCGCCACCTACGAAGAG |
| | AACCGTGCCGAGGCCCAGCGCTGGGCCACTGCCCTCACCTGTCTGCTCC |
| | GAGGACTGCCACTGCCCGGGGATGGGGAGATCACCCCTGACCTGCTAC |
| | CTCGGCCGCCCCGGTTGCTTCTATTGGTCAATCCCTTTGGGGGTCGGGG |
| | CCTGGCCTGGCAGTGGTGTAAGAACCACGTGCTTCCCATGATCTCTGAA |
| | GCTGGGCTGTCCTTCAACCTCATCCAGACAGAACGACAGAACCACGCC |
| | CGGGAGCTGGTCCAGGGGCTGAGCCTGAGTGAGTGGGATGGCATCGTC |
| | ACGGTCTCGGGAGACGGGCTGCTCCATGAGGTGCTGAACGGGCTCCTA |
| | GATCGCCCTGACTGGGAGGAAGCTGTGAAGATGCCTGTGGGCATCCTC |
| | CCCTGCGGCTCGGGCAACGCGCTGGCCGGAGCAGTGAACCAGCACGGG |
| | GGATTTGAGCCAGCCCTGGGCCTCGACCTGTTGCTCAACTGCTCACTGT |
| | TGCTGTGCCGGGGTGGTGGCCACCCACTGGACCTGCTCTCCGTGACGCT |
| | GGCCTCGGGCTCCCGCTGTTTCTCCTTCCTGTCTGTGGCCTGGGGCTTCG |
| | TGTCAGATGTGGATATCCAGAGCGAGCGCTTCAGGGCCTTGGGCAGTG |
| | CCCGCTTCACACTGGGCACGGTGCTGGGCCTCGCCACACTGCACACCTA |
| | CCGCGGACGCCTCTCCTACCTCCCCGCCACTGTGGAACCTGCCTCGCCC |
| | ACCCCTGCCCATAGCCTGCCTCGTGCCAAGTCGGAGCTGACCCTAACCC |
| | CAGACCCAGCCCCGCCCATGGCCCACTCACCCCTGCATCGTTCTGTGTC |
| | TGACCTGCCTCTTCCCCTGCCCCAGCCTGCCCTGGCCTCTCCTGGCTCGC |
| | CAGAACCCCTGCCCATCCTGTCCCTCAACGGTGGGGGCCCAGAGCTGGC |
| | TGGGGACTGGGGTGGGGCTGGGGATGCTCCGCTGTCCCCGGACCCACT |
| | GCTGTCTTCACCTCCTGGCTCTCCCAAGGCAGCTCTACACTCACCCGTCT |
| | CCGAAGGGGCCCCCGTAATTCCCCCATCCTCTGGGCTCCCACTTCCCAC |
| | CCCTGATGCCCGGGTAGGGGCCTCCACCTGCGGCCCGCCCGACCACCTG |
| | CTGCCTCCGCTGGGCACCCCGCTGCCCCCAGACTGGGTGACGCTGGAGG |
| | GGGACTTTGTGCTCATGTTGGCCATCTCGCCCAGCCACCTAGGCGCTGA |
| | CCTGGTGGCAGCTCCGCATGCGCGCTTCGACGACGGCCTGGTGCACCTG |
| | TGCTGGGTGCGTAGCGGCATCTCGCGGGCTGCGCTGCTGCGCCTTTTCT |
| | TGGCCATGGAGCGTGGTAGCCACTTCAGCCTGGGCTGTCCGCAGCTGGG |
| | CTACGCCGCGGCCCGTGCCTTCCGCCTAGAGCCGCTCACACCACGCGGC |
| | GTGCTCACAGTGGACGGGGAGCAGGTGGAGTATGGGCCGCTACAGGCA |
| | CAGATGCACCCTGGCATCGGTACACTGCTCACTGGGCCTCCTGGCTGCC |
| | CGGGGGGGGAGCCCTGA (SEQ ID NO: 25) |
| CerK | ATGGGGGCGACGGGGGCGGCGGAGCCGCTGCAATCCGTGCTGTGGGTG |
| | AAGCAGCAGCGCTGCGCCGTGAGCCTGGAGCCCGCGCGGGCTCTGCTG |
| | CGCTGGTGGCGGAGCCCGGGGCCCGGAGCCGGCGCCCCCGGCGCGGAT |
| | GCCTGCTCTGTGCCTGTATCTGAGATCATCGCCGTTGAGGAAACAGACG |
| | TTCACGGGAAACATCAAGGCAGTGGAAAATGGCAGAAAATGGAAAAG |
| | CCTTACGCTTTTACAGTTCACTGTGTAAAGAGAGCACGACGGCACCGCT |
| | GGAAGTGGGCGCAGGTGACTTTCTGGTGTCCAGAGGAGCAGCTGTGTC |
| | ACTTGTGGCTGCAGACCCTGCGGGAGATGCTGGAGAAGCTGACGTCCA |
| | GACCAAAGCATTTACTGGTATTTATCAACCCGTTTGGAGGAAAAGGAC |
| | AAGGCAAGCGGATATATGAAAGAAAAGTGGCACCACTGTTCACCTTAG |
| | CCTCCATCACCACTGACATCATCGTTACTGAACATGCTAATCAGGCCAA |
| | GGAGACTCTGTATGAGATTAACATAGACAAATACGACGGCATCGTCTG |
| | TGTCGGCGGAGATGGTATGTTCAGCGAGGTGCTGCACGGTCTGATTGGG |
| | AGGACGCAGAGGAGCGCCGGGGTCGACCAGAACCACCCCCGGGCTGTG |
| | CTGGTCCCCAGTAGCCTCCGGATTGGAATCATTCCCGCAGGGTCAACGG |
| | ACTGCGTGTGTTACTCCACCGTGGGCACCAGCGACGCAGAAACCTCGG |
| | CGCTGCATATCGTTGTTGGGGACTCGCTGGCCATGGATGTGTCCTCAGT |
| | CCACCACAACAGCACACTCCTTCGCTACTCCGTGTCCCTGCTGGGCTAC |
| | GGCTTCTACGGGGACATCATCAAGGACAGTGAGAAGAAACGGTGGTTG |
| | GGTCTTGCCAGATACGACTTTTCAGGTTTAAAGACCTTCCTCTCCCACC |
| | ACTGCTATGAAGGGACAGTGTCCTTCCTCCCTGCACAACACACGGTGGG |
| | ATCTCCAAGGGATAGGAAGCCCTGCCGGGCAGGATGCTTTGTTTGCAG |
| | GCAAAGCAAGCAGCAGCTGGAGGAGGAGCAGAAGAAAGCACTGTATG |
| | GTTTGGAAGCTGCGGAGGACGTGGAGGAGTGGCAAGTCGTCTGTGGGA |
| | AGTTTCTGGCCATCAATGCCACAAACATGTCCTGTGCTTGTCGCCGGAG |
| | CCCCAGGGGCCTCTCCCCGGCTGCCCACTTGGGAGACGGGTCTTCTGAC |
| | CTCATCCTCATCCGGAAATGCTCCAGGTTCAATTTTCTGAGATTTCTCAT |
| | CAGGCACACCAACCAGCAGGACCAGTTTGACTTCACTTTTGTTGAAGTT |
| | TATCGCGTCAAGAAATTCCAGTTTACGTCGAAGCACATGGAGGATGAG |
| | GACAGCGACCTCAAGGAGGGGGGGAAGAAGCGCTTTGGGCACATTTGC |
| | AGCAGCCACCCCTCCTGCTGCTGCACCGTCTCCAACAGCTCCTGGAACT |
| | GCGACGGGGAGGTCCTGCACAGCCCTGCCATCGAGGTCAGAGTCCACT |
| | GCCAGCTGGTTCGACTCTTTGCACGAGGAATTGAAGAGAATCCGAAGC |
| | CAGACTCACACAGCTGA(SEQ ID NO: 26) |

TABLE 3
Amino Acid Sequences

| | |
|---|---|
| ASAH1 | MPGRSCVALVLLAAAVSCAVAQHAPPWTEDCRKSTYPPSGPTYRGAVPWY |
| transcript | TINLDLPPYKRWHELMLDKAPVLKVIVNSLKNMINTFVPSGKIMQVVDEKLP |
| variant 1 | GLLGNFPGPFEEEMKGIAAVTDIPLGEIISFNIFYELFTICTSIVAEDKKGHLIHG |
| (ACv1) | RNMDFGVFLGWNINNDTWVITEQLKPLTVNLDFQRNNKTVFKASSFAGYVG |
| | MLTGFKPGLFSLTLNERFSINGGYLGILEWILGKKDVMWIGFLTRTVLENSTS |
| | YEEAKNLLTKTKILAPAYFILGGNQSGEGCVITRDRKESLDVYELDAKQGRW |
| | YVVQTNYDRWKHPFFLDDRRTPAKMCLNRTSQENISFETMYDVLSTKPVLN |
| | KLTVYTTLIDVTKGQFETYLRDCPDPCIGW (SEQ ID NO: 27) |
| ASAH1 | MNCCIGLGEKARGSHRASYPSLSALFTEASILGFGSFAVKAQWTEDCRKSTY |
| transcript | PPSGPTYRGAVPWYTINLDLPPYKRWHELMLDKAPVLKVIVNSLKNMINTFV |
| variant 2 | PSGKIMQVVDEKLPGLLGNFPGPFEEEMKGIAAVTDIPLGEIISFNIFYELFTIC |
| (ACv2) | TSIVAEDKKGHLIHGRNMDFGVFLGWNINNDTWVITEQLKPLTVNLDFQRN |
| | NKTVFKASSFAGYVGMLTGFKPGLFSLTLNERFSINGGYLGILEWILGKKDV |
| | MWIGFLTRTVLENSTSYEEAKNLLTKTKILAPAYFILGGNQSGEGCVITRDRK |
| | ESLDVYELDAKQGRWYVVQTNYDRWKHPFFLDDRRTPAKMCLNRTSQENI |
| | SFETMYDVLSTKPVLNKLTVYTTLIDVTKGQFETYLRDCPDPCIGW (SEQ ID |
| | NO: 28) |
| ASAH1 | MNCCIGLGEKARGSHRASYPSLSALFTEASILGFGSFAVKAQWTEDCRKSTY |
| transcript | PPSGPTVFPAVIRYRGAVPWYTINLDLPPYKRWHELMLDKAPVPGLLGNFPG |
| variant 3 | PFEEEMKGIAAVTDIPLGEIISFNIFYELFTICTSIVAEDKKGHLIHGRNMDFGV |
| | FLGWNINNDTWVITEQLKPLTVNLDFQRNNKTVFKASSFAGYVGMLTGFKP |
| | GLFSLTLNERFSINGGYLGILEWILGKKDVMWIGFLTRTVLENSTSYEEAKNL |
| | LTKTKILAPAYFILGGNQSGEGCVITRDRKESLDVYELDAKQGRWYVVQTN |
| | YDRWKHPFFLDDRRTPAKMCLNRTSQENISFETMYDVLSTKPVLNKLTVYT |
| | TLIDVTKGQFETYLRDCPDPCIGW (SEQ ID NO: 29) |

In one embodiment, the acid ceramidase is encoded by a nucleic acid selected from SEQ ID NO: 8, SEQ ID NO: 13, and SEQ ID NO: 14. Alternatively, the method may be used to code for expression of a ceramidase that is a neutral ceramidase or an alkaline ceramidase. The acidic, neutral, or alkaline ceramidase may be encoded by a nucleic acid selected from any of SEQ ID NO: 8, SEQ ID NO: 13, SEQ ID NO: 14, SEQ ID NO: 15, SEQ ID NO: 16, SEQ ID NO: 17, SEQ ID NO: 18, or SEQ ID NO: 19.

The sample can be screened for ceramidase activity, protein level, or nucleic acid encoding level by methods that will be apparent to the skilled artisan. Suitable methods include, for example, AC activity assays (Eliyahu et al., “Acid Ceramidase Is a Novel Factor Required for Early Embryo Survival,” FASEB J. 21(7):1403-9 (2007), which is hereby incorporated by reference in its entirety), western blotting to determine the relative amount of AC present in the sample (where a higher amount of AC protein correlates to a higher AC activity level) (Eliyahu et al., “Acid Ceramidase Is a Novel Factor Required for Early Embryo Survival,” FASEB J. 21(7):1403-9 (2007), which is hereby incorporated by reference in its entirety), and RIA (Ferlinz et al., “Human Acid Ceramidase: Processing, Glycosylation, and Lysosomal Targeting,” J. Biol. Chem. 276(38):35352-60 (2001), which is hereby incorporated by reference in its entirety).

The present disclosure, in one embodiment, may relate to polypeptides with conservative amino acid substitutions, insertions, and/or deletions with respect to the ceramidase sequence. “Conservative” as used herein includes that the alterations are as conformationally neutral as possible, i.e., they are designed to produce minimal changes in the tertiary structure of the mutant polypeptides as compared to the ceramidase, and that the changes are as antigenically neutral as possible, i.e., they are designed to produce minimal changes in the antigenic determinants of the mutant polypeptides as compared to the ceramidase. Conformational neutrality may be desirable for preserving biological activity, and antigenic neutrality may be desirable for avoiding the triggering of immunogenic responses in subjects treated with the compounds of the present disclosure. Guidelines exist which can allow those skilled in the art to make alterations that have high probabilities of being conformationally and antigenically neutral if desired. Some of the those guidelines include: substitution of hydrophobic residues is less likely to produce changes in antigenicity because they are likely to be located in the protein's interior; substitution of physiochemically similar residues has a lower likelihood of producing conformational changes because the substituting amino acid can play the same structural role as the replaced amino acid; alteration of evolutionarily conserved sequences is likely to produce deleterious conformational effects because evolutionary conservation suggests sequences may be functionally important, and negatively charged residues, for example, Asp and Glu, tend to be more immunogenic than neutral or positively charged residues (see Geysen et al., “Chemistry of Antibody Binding to a Protein,” Science 235:1184-90 (1987), which is hereby incorporated by reference in its entirety).

An acid ceramidase may have an amino acid sequence homology that is 80% or higher to SEQ ID NO: 27, SEQ ID NO: 28, or SEQ ID NO: 29. In another example, an acid ceramidase may have an amino acid sequence homology that is 81% or higher, or 82% or higher, or 83% or higher, or 84% or higher, or 85% or higher, or 86% or higher, or 87% or higher, or 88% or higher, or 89% or higher, or 90% or higher, or 91% or higher, or 92% or higher, or 93% or higher, or 94% or higher, 95% or higher, or 96% or higher, or 97% or higher, or 98% or higher, or 99% or higher to SEQ ID NO: 27, SEQ ID NO: 28, or SEQ ID NO: 29. In another example, an acid ceramidase may have an amino acid sequence that is 80%, 81%, 82%, 83%, 84%, 85%, 86%, 87%, 88%, 89%, 90%, 91%, 92%, 93%, 94%, 95%, 96%, 97%, 98%, 99% or 100% homologous to SEQ ID NO: 27, SEQ ID NO: 28, or SEQ ID NO: 29.

An acid ceramidase may have an amino acid sequence similarity that is 80% or higher to SEQ ID NO: 27, SEQ ID NO: 28, or SEQ ID NO: 29. In another example, an acid ceramidase may have an amino acid sequence similarity that is 81% or higher, or 82% or higher, or 83% or higher, or 84% or higher, or 85% or higher, or 86% or higher, or 87% or higher, or 88% or higher, or 89% or higher, or 90% or higher, or 91% or higher, or 92% or higher, or 93% or higher, or 94% or higher, 95% or higher, or 96% or higher, or 97% or higher, or 98% or higher, or 99% or higher to SEQ ID NO: 27, SEQ ID NO: 28, or SEQ ID NO: 29. In another example, an acid ceramidase may have an amino acid sequence that is 80%, 81%, 82%, 83%, 84%, 85%, 86%, 87%, 88%, 89%, 90%, 91%, 92%, 93%, 94%, 95%, 96%, 97%, 98%, 99% or 100% similar to SEQ ID NO: 27, SEQ ID NO: 28, or SEQ ID NO: 29.

There is similarity in function and sequence between acid ceramidases in different organisms and acid ceramidase from other species may have the same beneficial effect as presently disclosed. Acid ceramidase may be derived from a human, dog, cat, mouse, horse, pig, sheep, primate, fish, mammal or be a synthetic construct derived from one or more organism.

The methods described herein may be carried out by administering a therapeutically effective amount of an adeno-associated viral vector that codes for expression of acid ceramidase. Typically, a vector will be administered to a subject in a vehicle that delivers the vector to the target cell, tissue, or organ. The vector may be in the form of a composition.

The composition may further include a pharmaceutically acceptable carrier. “Pharmaceutically acceptable carriers” as used herein refer to conventional pharmaceutically acceptable carriers See Remington's Pharmaceutical Sciences, by E. W. Martin, Mack Publishing Co., Easton, Pa., 15th Edition (1975), which is hereby incorporated by reference in its entirety, describes compositions suitable for pharmaceutical delivery of the inventive compositions described herein. In particular, a pharmaceutically acceptable carrier as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits. In one embodiment, the pharmaceutically acceptable carrier is selected from the group consisting of a liquid filler, a solid filler, a diluent, an excipient, a solvent, and an encapsulating material.

Pharmaceutically acceptable carriers (e.g., additives such as diluents, immunostimulants, adjuvants, antioxidants, preservatives and solubilizing agents) are non-toxic to the cell or subject being exposed thereto at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; adjuvants (selected so as to avoid adjuvant-induced toxicity, such as a (3-glucan as described in U.S. Pat. No. 6,355,625, which is hereby incorporated by reference in its entirety, or a granulocyte colony stimulating factor (GCSF)); hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

In addition, in various embodiments, the compositions according to the disclosure may be formulated for delivery via any route of administration. The route of administration may refer to any administration pathway known in the art, including but not limited to aerosol, nasal, oral, transmucosal, transdermal, subcutaneous, or parenteral. Parenteral refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or in the form of lyophilized powders. In one embodiment, the administering is carried out by intravenous injection, intramuscular injection, subcutaneous injection, intradermal injection, intraosseous injection, localized injection, long-acting injection formulation, depot injection, implantable injection, endotracheal injection, intra-arterial injection, intrathecal, or any combination thereof. In one preferred embodiment, the administering includes a single administration of said adeno-associated viral vector.

In one embodiment, the composition may further include an adjuvant. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles. In one embodiment, the composition is formulated for the diagnosis and treatment of CD.

The composition may be orally administered, for example, with an inert diluent, or with an assimilable edible carrier, or they may be enclosed in hard or soft shell capsules, or they may be compressed into tablets, or may be incorporated directly with the food of the diet. For oral therapeutic administration, the composition may be incorporated with excipients and used in the form of tablets, capsules, elixirs, suspensions, syrups, and the like. Such compositions and preparations should contain at least 0.1% vector. The percentage of vector in these compositions may, of course, be varied and may conveniently be between about 2% to about 60% of the weight of the unit. The amount of vector in such therapeutically useful compositions is such that a suitable dosage will be obtained. In one embodiment, the adeno-associated viral vector is administered in an amount between about 1¹⁰ and about 1¹⁴ particles per joint.

The tablets, capsules, and the like may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

The vector may also be administered parenterally. Solutions or suspensions of ceramidase can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The vector may also be administered directly to the airways in the form of an aerosol. For use as aerosols, vector in solution or suspension may be packaged in a pressurized aerosol container together with suitable propellants, for example, hydrocarbon propellants like propane, butane, or isobutane with conventional adjuvants. The vector may also be administered in a non-pressurized form.

Exemplary delivery devices include, without limitation, nebulizers, atomizers, liposomes (including both active and passive drug delivery techniques) (Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987); Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al; U.S. Pat. No. 5,885,613 to Holland et al; U.S. Pat. No. 5,631,237 to Dzau & Kaneda; and U.S. Pat. No. 5,059,421 to Loughrey et al; Wolff et al, “The Use of Monoclonal Anti-Thy1 IgG1 for the Targeting of Liposomes to AK-A Cells in Vitro and in Vivo,” Biochim. Biophys. Acta 802:259-73 (1984), each of which is hereby incorporated by reference in its entirety), transdermal patches, implants, implantable or injectable protein depot compositions, and syringes. Other delivery systems which are known to those of skill in the art can also be employed to achieve the desired delivery of ceramidase to the desired organ, tissue, or cells.

Administration can be carried out as frequently as required and for a duration that is suitable to provide effective treatment. For example, administration can be carried out with a single sustained-release dosage formulation or with multiple daily doses. Alternatively, administration can be carried out with a single non-sustained-release dosage formulation or with a single daily dose. Administration of a single dose directly to the target area is a unique and useful feature of the compositions and methods described herein. The target area as described herein includes, but is not limited to, any damaged fluid, organ, tissue, cell, or any combination thereof. The target area as described herein further includes, but is not limited to, any area in a subject having or suspected of having inflammation, for example tissue inflammation, tissue damage, or a combination thereof.

The amount to be administered will, of course, vary depending upon the treatment regimen. The dose required to obtain an effective amount may vary depending on the agent, formulation, and individual to whom the agent is administered.

Typically, the vector will be administered as a pharmaceutical formulation that includes ceramidase and any pharmaceutically acceptable adjuvants, carriers, excipients, and/or stabilizers, and can be in solid or liquid form, such as tablets, capsules, powders, solutions, suspensions, or emulsions. The compositions preferably contain from about 0.01 to about 99 weight percent, more preferably from about 2 to about 60 weight percent, of the vector together with the adjuvants, carriers and/or excipients. In some embodiments, an effective amount ranges from about 0.001 mg/kg to about 500 mg/kg body weight of the subject. In some embodiments, the effective amount of the agent ranges from about 0.05 mg/kg to about 30 mg/kg, from about 0.1 mg/kg to about 30 mg/kg, from about 1 mg/kg to about 25 mg/kg, from about 1 mg/kg to about 20 mg/kg, or from about 1 or 2 mg/kg to about 15 mg/kg.

For purposes of this and other aspects of the disclosure, the target “subject” encompasses any vertebrate, such as an animal, preferably a mammal, more preferably a human. The target subject may encompass any subject that would benefit from repair to damaged fluid, organ, tissue, and/or cell; and the target subject may encompass any subject that has or is at risk of having an inflammation condition, particularly one that is associated with tissue damage. Particularly susceptible subjects include adults and elderly adults. However, any infant, juvenile, adult, or elderly adult that has or is at risk of having fluid, organ, tissue, and/or call damage, and/or an inflammation condition can be treated in accordance with the methods of the present disclosure. In one embodiment, the subject is an infant, a juvenile, or an adult.

The method of repairing damaged fluid, organ, tissue, and/or cell may further include selecting a subject having or susceptible of having an inflammation condition associated with tissue damage. The inflammation condition may include any condition, disease, or disorder that involved inflammation of one or more organ, tissue, cell, or any combination thereof. In one embodiment, the inflammation condition associated with tissue damage including spinal inflammation, joint inflammation, liver inflammation, fibrosis, or any combination thereof.

In one embodiment, the method of repairing damaged fluid, organ, tissue, and/or cell may further include selecting a subject having or susceptible of having an orthopedic condition and/or orthopedic injury. The orthopedic condition and/or orthopedic injury may include any injury to the bones, joints, and/or soft tissue. In one embodiment, the orthopedic condition and/or orthopedic injury is a joint condition and/or a joint injury. In one embodiment, the orthopedic condition and/or orthopedic injury including arthritis, bursitis, a degenerative joint and bone disease, fibromyalgia, a fracture, back pain, hand pain, knee pain, neck pain, hip pain, shoulder pain, kyphosis, a joint dislocation, a joint tear, a joint fracture, a joint break, gout, osteoporosis, Paget's disease, rheumatic disorder, a joint sprain, a joint strain, scoliosis, tendonitis, soft tissue injury, or any combination thereof. In one embodiment, the orthopedic condition and/or orthopedic injury including a degenerative joint and bone disease, said disease is selected from osteoarthritis, rheumatoid arthritis, psoriatic arthritis, ankylosing spondylitis, or any combination thereof.

As used herein, the phrase “therapeutically effective amount” means an amount that elicits the biological or medicinal response that is being sought in a tissue, system, animal, individual or human by a researcher, veterinarian, medical doctor, or other clinician. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment. It will also depend on the degree, severity, and type of disease or disorder. The skilled artisan will be able to determine appropriate dosages depending on these and other factors. The term “treatment” or “treat” may include effective inhibition, suppression, cessation, prevention of inflammation symptoms, or any combination of the foregoing, so as to prevent or delay the onset, retard the progression, or ameliorate the symptoms of inflammation.

Dosage, toxicity, and therapeutic efficacy of therapeutic agents and/or cells can be determined by standard pharmaceutical procedures in cell cultures or experimental animals, e.g., for determining the LD₅o (the dose lethal to 50% of the population) and the ED₅o (the dose therapeutically effective in 50% of the population). The dose ratio between toxic and therapeutic effects is the therapeutic index and it can be expressed as the ratio LD₅o/ED₅o. Agents/cells which exhibit high therapeutic indices may be desirable. While agents/cells that exhibit toxic side effects may be used, care should be taken to design a delivery system that targets such agents/cells to the site of affected tissue in order to minimize potential damage to uninfected cells and, thereby, reduce side effects.

Therapeutic agents and/or cells are administered to a subject in an amount to treat the disease or disorder. The amount is a quantity sufficient to achieve a desired therapeutic and/or prophylactic effect, e.g., an amount which results in the prevention of, or a decrease in, the cause or symptoms associated with the disease or disorder that is being treated. One goal of treatment is the amelioration, either partial or complete, either temporary or permanent, of patient symptoms, including inflammation. Any amelioration is considered successful treatment. This is especially true as amelioration of some magnitude may allow reduction of other medical or surgical treatment which may be more toxic or invasive to the patient.

As used herein, the term “simultaneous” therapeutic use refers to the administration of at least one additional agent beyond the adeno-associated viral vector that codes for expression of acid ceramidase, for example, agents administered before, during, or after the vector, optionally, by the same route and at the same time or at substantially the same time. As used herein, the term “separate” therapeutic use refers to an administration of at least one additional agent beyond the adeno-associated viral vector that codes for expression of acid ceramidase, for example, agents administered before, during, or after administration of a vector, at the same time or at substantially the same time by different routes. As used herein, the term “sequential” therapeutic use refers to administration of at least one additional agent beyond the adeno-associated viral vector that codes for expression of acid ceramidase, for example, agents administered before, during, or after administration of the vector at different times, the administration route being identical or different. More particularly, sequential use refers to the whole administration of the additional agent before administration of adeno-associated viral vector that codes for expression of acid ceramidase. It is thus possible to administer the additional agent over several minutes, hours, or days before applying the vector. In one embodiment, the additional agent is administered before, during, or after the vector.

A second aspect of the present disclosure relates to a method of reducing and/or preventing inflammation. The method includes selecting a subject having or susceptible of having an inflammation condition associated with tissue damage; administering a therapeutically effective amount of an adeno-associated viral vector that codes for expression of acid ceramidase to a damaged fluid, organ, tissue, and/or cell of the subject under conditions effective to reduce and/or prevent inflammation.

Inflammation as described herein includes any condition, disorder, or disease associated with tissue damage. In one embodiment, the inflammation condition associated with tissue damage including spinal inflammation, joint inflammation, liver inflammation, fibrosis, lupus, or any combination thereof. In one embodiment, the inflammation condition is an orthopedic condition and/or orthopedic injury.

This aspect of the disclosure is carried out in accordance with the previously described aspect.

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense. The present disclosure may be further illustrated by reference to the following examples.

### EXAMPLES

The following examples are intended to illustrate, but by no means are intended to limit, the scope of the present disclosure as set forth in the appended claims.

### Example 1—Materials and Methods

Animals were induced by IV injection of Propofol. Anesthesia is maintained by isoflurane gas administration via mask, followed by intubation and maintenance with isoflurane in conjunction with a ventilator and an endotracheal tube. Pain management will be supported by fentanyl patch and buprenorphine. Isoflurane anesthesia will be maintained throughout surgical procedures with depth of anesthesia monitored by vital signs, eye response to light, and response to physical manipulation. The affected hind limb was clipped free of hair, aseptically prepared, and draped. After incision exploration of the stifle joint was performed. The cranial cruciate ligament was debrided with a shaver, the joint was placed in a drawer position via external manipulation, and the menisci were evaluated by use of a probe during direct observation. Meniscal tears were debrided, or a medial caudal pole meniscectomy was performed if meniscal damage was severe. Post-surgery the Anc80 Acid Ceramidase injected intra-articular. The dog was validated clinically by at the tertiary hospital post 24 hours and every few days for clinical validation and blood work.

The term “pain score” is based on the Canine Brief Pain Inventory (Canine BPI) which allows one to rate the severity of the dog's pain and the degree to which that pain interferes with function. Initially developed to assess pain related to osteoarthritis, the Canine BPI has been shown to be an appropriate measure for pain caused by bone cancer as well. Canine BPI items are presented with 0-10 numerical rating scales where 0=no pain and 10=extreme pain.

The term “ambulatory score” is used to define normal fore limb, and hind limb coordination in dogs walking on a treadmill and then determine whether reliable data could be generated on the frequency of hind limb stepping and the frequency of coordinated stepping in dogs with a wide range of severities of injury. Scores are from grade 1-5. For example: Grade 2, Ambulatory Paraparesis, these dogs can walk but are weak and wobbly in the rear legs. They may cross their back legs when walking, splay out, knuckle over, or stumble in their back legs. Grade 3, non-ambulatory paraparesis, these dogs are still able to move their legs and wag their tails but are not strong enough to support their own weight and walk. Grade 5, intervertebral disc disease (IVDD) is the most severe stage of the condition. Dogs with stage 5 IVDD are paralyzed and have no deep pain sensation in their feet. If a dog cannot feel their toes, it will not walk or bear weight on its back legs. Therefore, a conservative approach is not the best option for dogs at this severe stage. The score is a deficit and as a result the score numbers decrease as the ambulation improves.

### Example 2—Research Plan and Field

The results suggest that Anc80.AC virus can be used to enhance soft tissue repair. In addition, there is a plan to apply the tissue repair Anc80.AC virus on other types orthopedic injury requiring tissue repair, such as cartilage repair.

The use of Anc80.AC has multiple advantages over other potential tissue repair technology such as cell therapy: Cell therapy that is used today usually involved a major surgery to retrieve stem cells usually fat tissue is collected from abdominal tissue. Post this surgery cell are cultured for several days and second procedure is done to administrate the cells to the injured area. This technology suggesting simple one-time injection of AC gene therapy into the injured area.

Physiological enzymes are expected to have no toxic effects. The AC protein is present as two forms (active and inactive) in the cell. The inactive AC protein undergoes an auto-self cleavage to the active from, which is responsible for hydrolyzing ceramide to sphingosine after exposure to stress. Transfecting cells with Anc80.AC will increase mostly the inactive precursor of the enzyme; this will allow physiological control to regulate the amount of the active AC protein required for survival. AC should not influence other cellular signaling, because the only known biological function of AC is the control of ceramide metabolism. In addition, a mouse model was created that is constantly overexpressing the AC enzyme (COEAC) in all tissues. The viability of COEAC mice proves the lack of toxicity by overexpressing the AC.

As mentioned, Anc80, an engineered gene therapy vector, is synthetic in nature and has been shown to reduce cross-reactivity with commonly used AAV vectors. Anc80 is a potent gene therapy vector that is not known to circulate in humans, making it less likely to be recognized immunologically by antibodies against naturally occurring AAVs. The present disclosure provides method to be used to accelerate ligament and cartilage repair by direct and easy gene delivery in animal and human orthopedic application.

### Example 3—Orthopedic Pre-Clinical Trial in Dog Anc80ACv1

Use of engineered viral vectors (gene therapy) encoding acid ceramidase enzyme as an easy and rapid tissue repair therapy.

The present disclosure provides a method for repairing tissue post-acute orthopedic injuries and post-surgery by administration of Anc80 virus encode to Acid Ceramidase (AC) to the joint. AC is a sole enzyme that can regulate ceramide hydrolysis to prevent cell death and to leads to the synthesis of Sphingosine 1 Phosphate (S1P) from Sphingosine to initiate cell survival. While using AC gene therapy as a survival factor that can prevent senescence and the progression of tissue damage and scarring it was discovered that AC expression initiate expression of factors that are leading to tissue repair. Previous studies in small and large animals to test the therapeutic potential of AC-based gene therapy for pulmonary arterial hypertension (PAH) in vivo, showed positive outcomes and exceeded expectations. The study using adeno-associated virus, Anc80L65, to deliver the AC gene cassette, demonstrated a complete lung recovery following administration of the therapeutic Anc80L65ACv1. By using an adeno-associated virus, Anc80, to deliver the AC gene cassette, complete lung recovery was demonstrated following a single administration of the therapeutic AC-Anc80. This study approved for dog clinical trial. The present disclosure suggests the use AC gene therapy to enhance tissue repair and organs recovery post orthopedic injury and surgeries. Anc80 virus, an in silico designed gene therapy vector, has demonstrated high gene expression levels in the liver, eye and ear compared to naturally-occurring adeno-associated viral vectors (AAVs) that are currently in clinical development. Zinn et al., “In Silico Reconstruction of the Viral Evolutionary Lineage Yields a Potent Gene Therapy Vector,” Cell Rep. 12:1056-1068 (2015), which is hereby incorporated by reference. AAV vectors, for transfecting cells with transcripts under the control of expression elements driving transcription of the transfected sequence. For example, plasmids for directing generation of AAV, such as Anc80L65, carrying polynucleotides for transcription, are commercially available and may be used to drive in vitro production AAV vectors. See, for example, a pAnc80L65 plasmid obtained from Addgene (plasmid #68837) and a pAAV.CMV.PI.EGFP.WPRE.bGH plasmid obtained from Addgene (plasmid #105530).

Anc80, an engineered gene therapy vector, is synthetic in nature it is not known to circulate in humans, making it less likely to be recognized immunologically by antibodies against naturally occurring AAVs. Orthopedic disorders are one of the leading causes of morbidity. Their prevalence will increase dramatically with age and gain weight. Many orthopedic conditions are good candidates for gene therapy.

In accordance with aspects of the present disclosure, AC gene therapy may improve the outcome of ligament rapture, bone fracture and cartilage damage in chronic disease. As disclosed herein, in a non-limiting example, injecting an AC-Anc80 to synovial fluid (non-Newtonian fluid found in the cavities of synovial joints) post-acute injury such as ligament rapture may initiate rapid ligament and cartilage repair. In an example, injection to the injured area may not require surgery or use of cell culture. This is a useful in vivo application to induce endogenous tissue repair alone or in combination with surgeries or cell therapy technic. It is clinically relevant to execute gene delivery in large animals and human and the Anc80 Acid Ceramidase addresses the problem of scale, whereby it is feasible to achieve very high gene transduction levels using surgical methods. Clinical study was conducted in dogs using Anc80.AC virus to repair tear ligament and cartilage repair in chronic disease. Results showed extremely fast recovery from ligament tear injury (two weeks). Inflammation, reduced in 50% as fast as 48 hours post treatment, less pain and more ambulatory. Blood work post 7 days conformed minimal inflammation and no toxicity. The progress of healing as reported by the orthopedic veterinarian was more than two times as fast.

Subject 1, Benjamin—Benjamin was diagnosed with Osteoarthritis (OA). OA is the most common form of arthritis in dogs, affecting approximately a quarter of the population. It is a chronic joint disease characterized by loss of joint cartilage, thickening of the joint capsule and new bone formation around the joint (osteophytosis) and ultimately leading to pain and limb dysfunction. Majority of OA in dog occur secondarily to developmental orthopedic disease, such as cranial cruciate ligament disease, hip dysplasia, elbow dysplasia, OCD, patella (knee cap) dislocation. Benjamin presented withsevere OA and had problems with walking, jumping, running and climbing upstairs for the last 5 years. Benjamin was subjected to several treatment but with no success to improve his condition. On Nov. 9, 2020 he was subjected to stem cell therapy and Anc80L65ACv1 gene therapy injection. During the major surgery to collect fat cells for the gene therapy Benjamin was injected with 0.6 ml Anc80L65ACv1(1.4×10 by 12/ml). Four days post injection (before stem cell treatment) Benjamin demonstrated major improvement that included climbing up stairs and jumping. Two weeks after injection Benjamin demonstrated full recovery from OA and his ability to run was very impressive as he couldn't run for five years prior to treatment. Five-month post treatment Benjamin keeps improving.

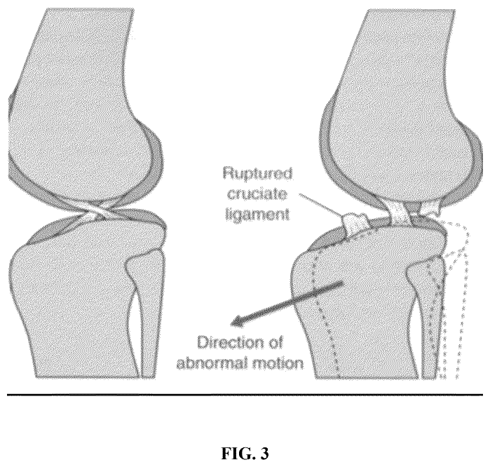

Subject 2, Nuset—ACL injury which is a tear or sprain of the anterior cruciate ligament (ACL). Cranial cruciate ligament (CCL) rupture is the tearing of an important ligament in the stifle joint (knee), resulting in partial or complete joint instability, pain, and lameness. If the injury is not treated, damage to connective tissues and degenerative joint disease often results. Such an injury is exemplified in FIG. 3.

Nuset was a 5-year-old that had a complete ACL injury in his right knee on Sep. 19, 2020. At spot on veterinary hospital Nuset had a clinical validation and X-ray and based on the injury an ACL surgery was suggested. On Sep. 21, 2020, ACL surgery was done by Dr. Putter Philip. Nuset was knuckling (dragging foot-top-down) and completely non-weight baring. Two days after the surgery sever inflammation was observed and the dog was subjected to Anc80L65ACv1 gene therapy injection. On September 23rd Dr. Putter injected 0.6 ml of Anc80L65ACv1 (1.4×10 by 12/ml). 24 hours post injection 50% reduction of inflammation observed. 48 hours post injection Inflammation reduced more and swelling was gone. Dog presented much less pain and was more ambulatory in one week. Blood was taken for cell blood count and chemistry for toxicity evaluation. Blood values were in the normal range and no toxicity observed. Post two weeks Nuset started to run with mild limping. Six months post treatment Nuset keeps doing impressively well.

Four representative clinical cases are detailed by clinical phenotype in Table 4.

TABLE 4
Sample Case Studies of Animals Treated

| Nauset S | Benjamin D |
|---|---|
| Neutered male 90 lb lab mix DOB Nov. 14, 2013. | DOB Jun. 10, 2016, neutered male lab, 80 lbs. |
| HX of Addisons dx (October 2017) | Bilat OA hips, Bilat knee pain, lumbar pain on |
| Anaplasmosis. TX with Zycortal and Pred | palpation. Ulnal/tibial tarsal joint inflammation |
| since 2017 | Won't do stairs, bunny hops with anything faster |
| Acute knee injury Sep. 15, 2020 non-weight | than a walk, obese. |
| bearing, Radiographs, physical exam under | Pain score: 7. |
| sedation, severe pyoderma L stifle culture | Nov. 9, 2020 CBC/Chem, radiographs hips/stifles |
| taken. Pain score: 8. | May 2020 CBC/Chem/T4 |
| Sep. 18, 2020 Surgeon consult/surgical repair | February 2020 CBC/Chem/T4 |
| full CCL tear medial meniscus intact, | AC injections Nov. 11, 2020 |
| severely thickened joint capsule with | Post- 4 days - running and jumping for first |
| moderate effusion. Radiographs taken | time in 4-5 years, pain score 0-1, continued |
| Sep. 23, 2020 AC injections | muscle regrowth over 4 months following |
| Resolution - advanced healing 6 days | |
| post full ambulaton x4, Sx site healed | |
| (typically 2 weeks), pain 2-3, long term | |
| little medial butress formed (OA | |
| secondary to surgery) | |
| Charlie G | Ticket Putter |
| 3 yr 10 mo, canine, | Weight 47.9, 14 yo, PE - weakness in hind end, |
| Pain (#/4): 2 - forelimbs - LF carpus 2/4, LF | paresis, falling down intermittently during |
| shoulder 1-2/4. RF shoulder 1/4 Forelimb | ambulation, difficulty rising, |
| lameness - chronic pain on flexion of LF | pain 4-5/10 spinal. Spinal rads and CT show |
| carpus, Pain - yelp L shoulder, Pain whine | IVDD, narrowing of disk space and spondylosis, |
| R Shoulder | Ch. Thoracic and cd lumbar vertebrae. Antinol. |
| Anaplasma positive - on doxycycline trial - | NSAIDS, Gabapentin helpful to decrease pain to |
| helpful per owner | 3-4 but CS no improvement in QOL, ambulation/ |
| Gaba/NSAID provide little to no relief | neuro signs no change - remain intermittent |
| CBC/SMA/UA Aug. 13, 2021 CT Jun. 18, 2021 | decreased Conscious proprioception. |
| AC injections Jun. 18, 2021 | AC Injections performed- Intra-thecal/hips/ |
| Post- 2 weeks post amb x 4 - had not | knees, |
| done so in months, running, bright, | 5 days post- jumping, falling ceased, amb x 4, |
| down stairs, jumping within 3 weeks | pain score 2-3, |
| post. Owner reports its “miraculous” | long term 2 months- liver values improved, |
| assuming this was the rest of this young | muscle regrowth. |
| dog life | Received total 2 injections AC - no reactions or |
| | side effects |

Table 5 shows a summary of ten patient results.

TABLE 5
Sample Subjects Studied

| | | | | | Pain score | Pain score/ |
|---|---|---|---|---|---|---|
| | Age | | Pain score | Locations | (5-10 days | Improvement of |
| Patient | (Years) | Disease treated | pre Tx | treated | post injection) | clinical signs |
| Nauset S | 7 | ACL complete | 8 | Left knee - | 2-3 | 5 days post- Healed |
| | | tear | | ACL surgical | | 2x faster |
| | | | | repair, AC | | |
| | | | | administered | | |
| | | | | during surgery | | |
| Jackson B | 2 | Bilat ACL | 7 | ACL partial | 1 | 9 days post- |
| | | complete tear, | | tear, surgical | | Attitude |
| | | chronic severe | | intervention | | improvement- |
| | | lameness (Stem | | avoided | | decreased |
| | | previous) | | | | aggression |
| Adamson | 8 | Chronic OA, 3 yr | 5 | Intra-theca, | 2 | Ambulation/ |
| H | | previous ACL | | Knees bilat, | | improved agility, |
| | | repair- chronic | | Hips Bilat | | neuro signs, |
| | | lameness, CT | | | | improving over |
| | | | | | | last 3 months |
| Charlie B | 10 | Chronic severe | 6-7 | Intra-theca, | 3 | Able to walk, run, |
| | | OA, Hind end | | Knees bilat, | | jump, |
| | | weakness and | | Hips Bilat | | improvement in |
| | | pain, unable to | | | | neuro signs, |
| | | rise, prepared | | | | muscle growth |
| | | to euthanize | | | | “miracle” per |
| | | | | | | owner |
| Charlie G | 2 | Chronic | 8 | Left shoulder, | 4 | 8 days post began |
| | | forelimb | | Right carpus | | to slow run, |
| | | lameness bilat, | | | | lethargy resolved, |
| | | 2yo dog, | | | | muscle regrowth, |
| | | lethargic, | | | | attitude |
| | | unable to run, | | | | improvement |
| | | Neurology | | | | “amazing” per |
| | | workup referral | | | | owner |
| | | and CT | | | | |
| Teddy L | 1 | Chronic Medial | 4 | Knees Bilat | 1-2 | 2 weeks post |
| | | patellar | | | | ambulation x 4 |
| | | Luxation and | | | | limbs, lameness |
| | | lameness Left | | | | only while |
| | | hind, 3 leg | | | | running - |
| | | lame | | | | intermittent hop - |
| | | | | | | avoid surgical |
| | | | | | | intervention |
| Duke O | 11 | Chronic | 6-8 | Intra-theca, | 4-5 | 8 days post - |
| | | progressive OA - | | Knees bilat, | | intermittent |
| | | Hip dysplasia, | | Hips Bilat | | jumping - first |
| | | IVDD, Knee | | | | time in years, |
| | | OA bilat | | | | reduced weakness |
| | | | | | | and neuro signs, |
| | | | | | | ambulation |
| | | | | | | improved, hoping |
| | | | | | | out of car |
| Rascal A | 11 | RH ACL partial | 7 | Knees bilat | 1-2 | 10-14 days post |
| | | Jan. 12, 2021 | | | | continued |
| | | | | | | improvement for |
| | | | | | | 2 months/ |
| | | | | | | ambulation x 4, |
| | | | | | | weight baring all |
| | | | | | | limbs |
| Andy V | 9 | Chronic OA, | 6-7 | Knees bilat | 3 | Return to |
| | | previous failed | | | | ambulation on 4 |
| | | surgery TAA for | | | | legs over 2 months |
| | | ACL tear, Right | | | | following |
| | | hind lameness, | | | | treatment |
| | | OA | | | | |
| Scout K | 10 | Chronic OA, | 5 | Intra-theca, | 2 | Decreased neuro |
| | | weakness in hind | | Knees bilat, | | signs, improved |
| | | end - spine/hips | | Hips Bilat | | walk/rise/run |
| | | pain/mild knee | | | | |
| | | OA bilat | | | | |

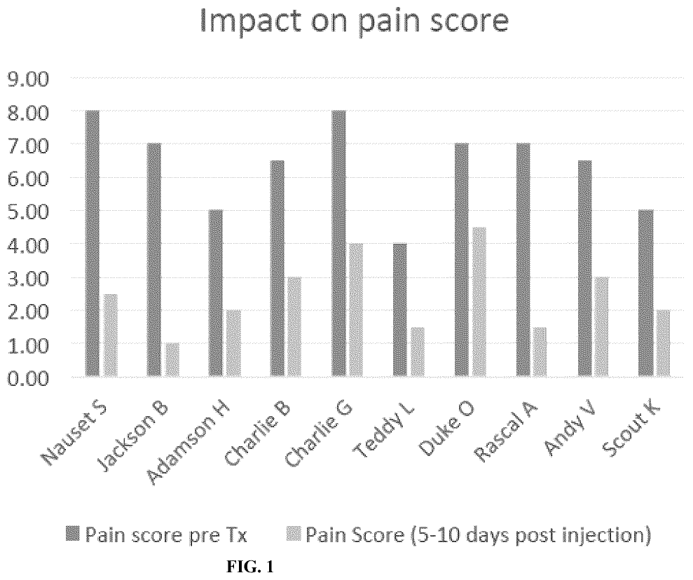

FIG. 1 shows impact on pain score on test subjects (before treatment and after treatment). FIG. 1 presents ten dogs that were treated with injections of adeno-associated virus encoding acid ceramidase. The Canine Brief Pain Inventory (Canine BPI) is a pain scale that allows one to rate the severity of the dog's pain and the degree to which that pain interferes with function. Canine BPI items are presented with a 0 to 10 numerical rating scale where 0 indicates no pain and 10 indicates extreme pain. The dogs had pain scores that ranged from 4 to 8 pre-treatment. The data indicates the pain scores for all dogs was reduced 5 to 10 days post treatment. The

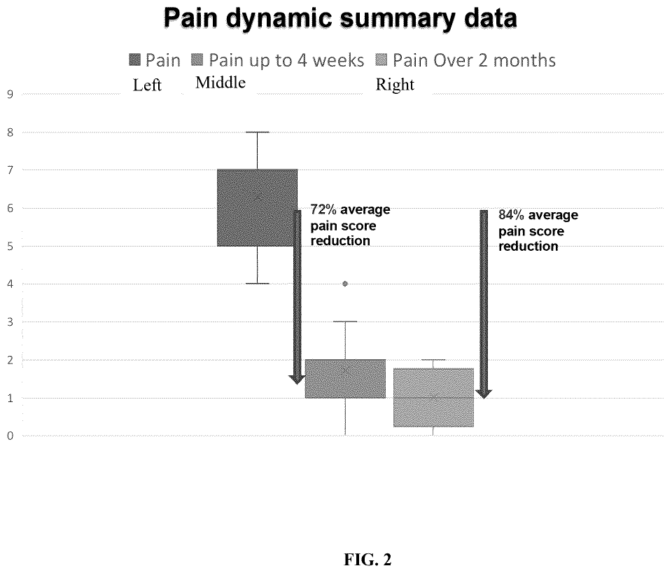

FIG. 2 shows pain dynamics summary data on test subjects (before treatment and after treatment). Fourteen dogs were evaluated prior to treatment, up to four weeks after treatment and over two months after treatment. The data indicate the largest decrease in pain was observed up to four weeks after treatment with a smaller decrease in pain even after two months. The treatments had a favorable safety profile, with no significant adverse events or negative impact on organ function. In addition to the decrease in pain, dogs saw significant improvement in joint inflammation and motility function.

FIG. 3 depicts a ligament injury that may be treated in accordance with the methods of the present disclosure. A cruciate ligament rupture is usually extremely painful and the knee joint becomes unstable, resulting in lameness. A more chronic form of cruciate damage occurs due to progressive weakening of the ligaments as a result of repeated trauma or arthritic disease.

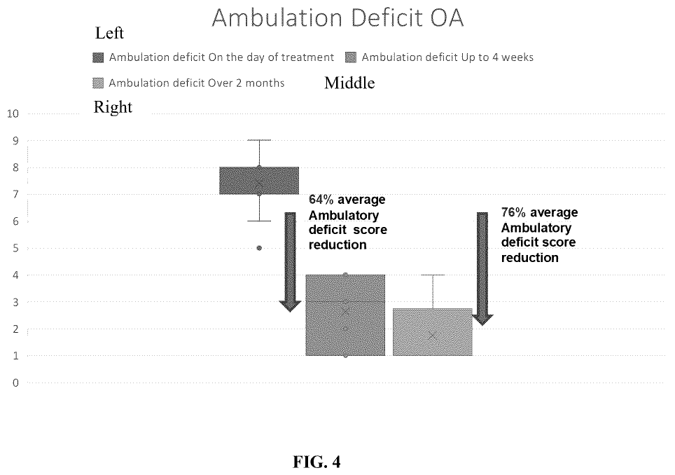

FIG. 4 illustrates an osteoarthritis ambulation deficit score on 14 test subjects on the day of treatment, up to four weeks after treatment, and over two months after treatment. The ambulatory score reports fore limb and hind limb coordination in dogs walking on a treadmill. The ambulation deficit score is on a scale of 1 to 5 where 5 is the most severe condition, such as paralysis. An example of a score of 2 is ambulatory paraparesis, these dogs can walk but are weak and wobbly in the rear legs. They may cross their back legs when walking, splay out, knuckle over, or stumble in their back legs.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating an orthopedic condition, an orthopedic injury, or both in a subject in need of such treatment, comprising:
administering a therapeutically effective amount of an adeno-associated viral vector to the subject, wherein the adeno-associated viral vector transfects cells with a polynucleotide sequence that encodes and drives expression of an acid ceramidase.

2. The method of claim 1 wherein one or both of the orthopedic condition and the orthopedic injury comprise inflammation.

3. The method of claim 1 or 2, wherein one or both of the orthopedic condition and the orthopedic injury comprise joint inflammation.

4. The method of any one of claims 1 to 3, wherein one or both of the orthopedic condition and the orthopedic injury comprises spinal inflammation.

5. The method of any one of claims 1 to 4, wherein one or both of the orthopedic condition and the orthopedic injury comprise a joint condition or a joint injury.

6. The method of any one of claims 1 to 5, wherein one or both of the orthopedic condition and the orthopedic injury comprises arthritis, bursitis, a degenerative joint and bone disease, fibromyalgia, a fracture, back pain, hand pain, knee pain, neck pain, hip pain, shoulder pain, kyphosis, a joint dislocation, a joint tear, a joint fracture, a joint break, gout, osteoporosis, Paget's disease, rheumatic disorder, a joint sprain, a joint strain, scoliosis, tendonitis, soft tissue injury, dysplasia, ligament injury, or any combination of two or more of the foregoing.

7. The method of any one of claims 1 to 6, wherein, when one or both of the orthopedic condition and the orthopedic injury comprises a degenerative joint and bone disease, said disease is selected from osteoarthritis, rheumatoid arthritis, psoriatic arthritis, ankylosing spondylitis, or any combination of two or more of the foregoing.

8. The method of any one of claims 1 to 7, wherein said administering comprises a single administration of said adeno-associated viral vector.

9. The method of any one of claims 1 to 8, wherein the administering comprises intravenous injection, intramuscular injection, subcutaneous injection, intradermal injection, intraosseous injection, localized injection, long-acting injection formulation, depot injection, implantable injection, endotracheal injection, intra-arterial injection, intrathecal, or any combination of two or more of the foregoing.

10. The method of any one of claims 1 to 9, wherein the adeno-associated viral vector is administered in an amount between about 1¹⁰ and about 1¹⁴ particles per administration.

11. The method of any one of claims 1 to 10, wherein the adeno-associated viral vector is selected from Anc80, Anc80L65, AAV6, AAV1, AAV2, AAV8, AAV9, or any combination of two or more of the foregoing.

12. The method of any one of claims 1 to 11, wherein the acid ceramidase comprises an amino acid sequence as set out in SEQ ID NO: 27, SEQ ID. NO: 28, or SEQ ID: 29.

13. The method of any one of claims 1 to 12, wherein the acid ceramidase comprises an amino acid sequence as set out in SEQ ID NO: 27.

14. A method of treating orthopedic inflammation in a subject in need of such treatment, comprising:
administering a therapeutically effective amount of an adeno-associated viral vector to the subject, wherein the adeno-associated viral vector transfects cells with a polynucleotide sequence that encodes and drives expression of an acid ceramidase.

15. The method of claim 14, wherein the inflammation comprises joint inflammation.

16. The method of claim 14 or 15, wherein the inflammation comprises spinal inflammation.

17. The method of any one of claims 14 to 16, wherein the orthopedic inflammation is caused by one or both of a joint condition and a joint injury.

18. The method of any one of claims 14 to 17, wherein the orthopedic inflammation comprises or is caused by arthritis, bursitis, a degenerative joint and bone disease, fibromyalgia, a fracture, back pain, hand pain, knee pain, neck pain, hip pain, shoulder pain, kyphosis, a joint dislocation, a joint tear, a joint fracture, a joint break, gout, osteoporosis, Paget's disease, rheumatic disorder, a joint sprain, a joint strain, scoliosis, tendonitis, soft tissue injury, dysplasia, ligament injury, or any combination of two or more of the foregoing.

19. The method of any one of claims 14 to 18, wherein the orthopedic inflammation is selected from osteoarthritis, rheumatoid arthritis, psoriatic arthritis, ankylosing spondylitis, or any combination of two or more of the foregoing.

20. The method of any one of claims 14 to 19, wherein said administering comprises a single administration of said adeno-associated viral vector.

21. The method of any one of claims 14 to 20, wherein said administering comprises intravenous injection, intramuscular injection, subcutaneous injection, intradermal injection, intraosseous injection, localized injection, long-acting injection formulation, depot injection, implantable injection, endotracheal injection, intra-arterial injection, intrathecal, or any combination of two or more of the foregoing.

22. The method of any one of claims 14 to 21, wherein said adeno-associated viral vector is administered in an amount between about 1¹⁰ and about 1¹⁴ particles per administration.

23. The method of any one of claims 14 to 22, wherein said adeno-associated viral vector is selected from Anc80, Anc80L65, AAV6, AAV1, AAV2, AAV8, AAV9, or any combination thereof.

24. The method of any one of claims 14 to 23, wherein the acid ceramidase comprises an amino acid sequence as set out in SEQ ID NO: 27, SEQ ID. NO: 28, or SEQ ID: 29.

25. The method of any one of claims 14 to 24, wherein the acid ceramidase comprises an amino acid sequence as set out in SEQ ID NO: 27.

26. The method of any one of claims 1 to 25, wherein the administering results in improved ambulation or mobility.

27. The method of any one of claims 1 to 26, wherein the administering results in pain reduction.

28. The method of any one of claims 1 to 27, wherein the administering results in faster wound healing than without the administering.


SEQUENCE LISTING (29 sequences)

SEQ ID NO: 1 - DNA, 4779 bp, synthetic construct
  source 1..4779: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcnnnggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 2 - DNA, 7446 bp, synthetic construct
  source 1..7446: mol_type = other DNA
agcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagacaggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttcagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacaaccaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccaacgaggaagaaattaaaaccaccaacccagtggccacagaagagtacggcacggtggccactaacctgcaatcggccaacaccgctcctgctacagggaccgtcaacagtcaaggagccttacctggcatggtctggcaggaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctgtaaactagtttgcttgttaatcaataaaccgtttaattcgtttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttcgcgcgcttggcgtaatcatggtcatagctgtttcctgtgtgaaattccacagcctggggtgcctaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagaaataataaaaaagccggattaataatctggctttttatattctctctctagtatataaacgcagaaaggcccacccgaaggtgagccagtgtgactctagtattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgagacggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagctctagtaaaataataaaaaagccggattaataatctggctttttatattctctctctagtatataaacgcagaaaggcccacccgaaggtgagccagtgtgacggcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcggcgaattgggtaccgggccccccctcgaggtcgacggtatcgtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcgga

SEQ ID NO: 3 - DNA, 7409 bp, Adeno-associated dependoparvovirus A
  source 1..7409: mol_type = other DNA
gctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttgattcgtttcagttgaactttggtctcctgtccttcttatcttatcggttaccatggttatagcttacacattaactgcttggttgcgcttcgcgataaaagacttacgtcatcgggttacccctagggggatccactagttctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcag

SEQ ID NO: 4 - DNA, 7468 bp, Adeno-associated dependoparvovirus A
  source 1..7468: mol_type = other DNA
acctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgccgatatcgttaacgccccgcgccggccgctctagaactagtggatcccccggaagatcagaagttcctattccgaagttcctattctctagaaagtataggaacttctgatctattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggagagtcctgtattagaggtcacgtgagtgttttgcgacattttgcgacaccatgtggtcacgctgggtatttaagcccgagtgagcacgcagggtctccattttgagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacg

SEQ ID NO: 5 - DNA, 7336 bp, Adeno-associated dependoparvovirus B
  source 1..7336: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgcc

SEQ ID NO: 6 - DNA, 7330 bp, Adeno-associated dependoparvovirus B
  source 1..7330: mol_type = other DNA
tcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccat

SEQ ID NO: 7 - DNA, 21829 bp, Adeno-associated dependoparvovirus B
  source 1..21829: mol_type = other DNA
ggatccgaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagggggatcctctagaactagtggatccgtccctagaagtaaaaaagggaaaaaagagtgtgtttgtcaaaataggagacaggtggtggcaaccagggacttataggggaccttacatctacagaccaacagatgcccccttaccatatacaggaagatatgacttaaattgggataggtgggtcacaatcaacggctataaagtgttatacagatccctcccctttcgtgaaagactcgccagagctagacctccttggtgtatgctaactgagaaagagaaagacgacatgaaacaacaggtacatgattatatttatctaggaacaggaatgcacttttggggaaaggttttccataccaaggaaggggcagtggctggactgatagaacattattctgcaaaaacttatggtatgagttattatgattagcctttatttgcccaaccttgcggttcccagggtttaaataagtttatggttacaaactgttcttaaaacaaggatgtgagacaagtggtttcctgagttggtttggtatcaaatgttctgatctgagctcttagtgttctattttcctatgttcttttggaatctatccaagtcttatgtaaatgcttatgtaaaccataatataaaagagtgctgattttttgagtaaacttgcaacagtcctaacattcttctctcgtgtgtttgtgtctgttcgccatcccgtctccgctcgtcacttatccttcacttttcagagggtccccccgcagatcccggtcaccctcaggtcgggacctgcagaagacgcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtctgttaatcaataaaccggttaattcgtgtcagttgaactttggtctcatgtcgttattatcttatctggtcaccatagcaatcgatgcatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttacaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgtctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccagggcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcctgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaagaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 8 - DNA, 1188 bp, Homo sapiens
  source 1..1188: mol_type = other DNA
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 9 - DNA, 1197 bp, Homo sapiens
  source 1..1197: mol_type = other DNA
atggatccagtggtcggttgcggacgtggcctctttggttttgttttctcagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatga

SEQ ID NO: 10 - DNA, 1062 bp, Homo sapiens
  source 1..1062: mol_type = other DNA
atgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtcatg

SEQ ID NO: 11 - DNA, 1653 bp, Photinus pyralis
  source 1..1653: mol_type = other DNA
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 12 - DNA, 801 bp, Aequorea victoria
  source 1..801: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagatccaaaaaagaagagaaaggtaggcgatccaaaaaagaagagaaaggtaggtgatccaaaaaagaagagaaaggtataa

SEQ ID NO: 13 - DNA, 1236 bp, Homo sapiens
  source 1..1236: mol_type = other DNA
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 14 - DNA, 1170 bp, Homo sapiens
  source 1..1170: mol_type = other DNA
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaactgtcttccctgctgttataaggtacagaggtgcagttccatggtacaccataaatcttgacttaccaccctacaaaagatggcatgaattgatgcttgacaaggcaccagtgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 15 - DNA, 2346 bp, Homo sapiens
  source 1..2346: mol_type = other DNA
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaaggaactctatgcctctgcctcccaggaggtaacaggaccactggcttcagcacaccagtgggtggatatgacagatgtgactgtctggctcaattccacacatgcatcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 16 - DNA, 2241 bp, Homo sapiens
  source 1..2241: mol_type = other DNA
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaagtcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 17 - DNA, 501 bp, Homo sapiens
  source 1..501: mol_type = other DNA
atgaggcagcatcgacaatttatggaccgcacgcattatctgcttacattcagctcttcagaaaccttgctaaggctattgctacgtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 18 - DNA, 972 bp, Homo sapiens
  source 1..972: mol_type = other DNA
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtgaatggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 19 - DNA, 483 bp, Homo sapiens
  source 1..483: mol_type = other DNA
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttag

SEQ ID NO: 20 - DNA, 795 bp, Homo sapiens
  source 1..795: mol_type = other DNA
atgcctagcatcttcgcctatcagagctccgaggtggactggtgtgagagcaacttccagtactcggagctggtggccgagttctacaacacgttctccaatatccccttcttcatcttcgggccactgatgatgctcctgatgcacccgtatgcccagaagcgctcccgctacatttacgttgtctgggtcctcttcatgatcataggcctgttctccatgtatttccacatgacgctcagcttcctgggccagctgctggacgagatcgccatcctgtggctcctgggcagtggctatagcatatggatgccccgctgctatttcccctccttccttggggggaacaggtcccagttcatccgcctggtcttcatcaccactgtggtcagcacccttctgtccttcctgcggcccacggtcaacgcctacgccctcaacagcattgccctgcacattctctacatcgtgtgccaggagtacaggaagaccagcaataaggagcttcggcacctgattgaggtctccgtggttttatgggctgttgctctgaccagctggatcagtgaccgtctgctttgcagcttctggcagaggattcatttcttctatctgcacagcatctggcatgtgctcatcagcatcaccttcccttatggcatggtcaccatggccttggtggatgccaactatgagatgccaggtgaaaccctcaaagtccgctactggcctcgggacagttggcccgtggggctgccctacgtggaaatccggggtgatgacaaggactgctga

SEQ ID NO: 21 - DNA, 828 bp, Homo sapiens
  source 1..828: mol_type = other DNA
atgggcgccccgcactggtgggaccagctgcaggctggtagctcggaggtggactggtgcgaggacaactacaccatcgtgcctgctatcgccgagttctacaacacgatcagcaatgtcttatttttcattttaccgcccatctgcatgtgcttgtttcgtcagtatgcaacatgcttcaacagtggcatctacttaatctggactcttttggttgtagtgggaattggatccgtctacttccatgcaacccttagtttcttgggtcagatgcttgatgaacttgcagtcctttgggttctgatgtgtgctttggccatgtggttccccagaaggtatctaccaaagatctttcggaatgaccggggtaggttcaaggtggtggtcagtgtcctgtctgcggttacgacgtgcctggcatttgtcaagcctgccatcaacaacatctctctgatgaccctgggagttccttgcactgcactgctcatcgcagagctaaagaggtgtgacaacatgcgtgtgtttaagctgggcctcttctcgggcctctggtggaccctggccctgttctgctggatcagtgaccgagctttctgcgagctgctgtcatccttcaacttcccctacctgcactgcatgtggcacatcctcatctgccttgctgcctacctgggctgtgtatgctttgcctactttgatgctgcctcagagattcctgagcaaggccctgtcatcaagttctggcccaatgagaaatgggccttcattggtgtcccctatgtgtccctcctgtgtgccaacaagaaatcatcagtcaagatcacgtga

SEQ ID NO: 22 - DNA, 804 bp, Homo sapiens
  source 1..804: mol_type = other DNA
atggctccggccgcggaccgagagggctactggggccccacgacctccacgctggactggtgcgaggagaactactccgtgacctggtacatcgccgagttctggaatacagtgagtaacctgatcatgattatacctccaatgttcggtgcagttcagagtgttagagacggtctggaaaagcggtacattgcttcttatttagcactcacagtggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 23 - DNA, 693 bp, Homo sapiens
  source 1..693: mol_type = other DNA
atggctccggccgcggaccgagagggctactggggccccacgacctccacgctggactggtgcgaggagaactactccgtgacctggtacatcgccgagttcttggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 24 - DNA, 519 bp, Homo sapiens
  source 1..519: mol_type = other DNA
atgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 25 - DNA, 1965 bp, Homo sapiens
  source 1..1965: mol_type = other DNA
atgaatggacaccttgaagcagaggagcagcaggaccagaggccagaccaggagctgaccgggagctggggccacgggcctaggagcaccctggtcagggctaaggccatggccccgcccccaccgccactggctgccagcaccccgctcctccatggcgagtttggctcctacccagcccgaggcccacgctttgccctcacccttacatcgcaggccctgcacatacagcggctgcgccccaaacctgaagccaggccccggggtggcctggtcccgttggccgaggtctcaggctgctgcaccctgcgaagccgcagcccctcagactcagcggcctacttctgcatctacacctaccctcggggccggcgcggggcccggcgcagagccactcgcaccttccgggcagatggggccgccacctacgaagagaaccgtgccgaggcccagcgctgggccactgccctcacctgtctgctccgaggactgccactgcccggggatggggagatcacccctgacctgctacctcggccgccccggttgcttctattggtcaatccctttgggggtcggggcctggcctggcagtggtgtaagaaccacgtgcttcccatgatctctgaagctgggctgtccttcaacctcatccagacagaacgacagaaccacgcccgggagctggtccaggggctgagcctgagtgagtgggatggcatcgtcacggtctcgggagacgggctgctccatgaggtgctgaacgggctcctagatcgccctgactgggaggaagctgtgaagatgcctgtgggcatcctcccctgcggctcgggcaacgcgctggccggagcagtgaaccagcacgggggatttgagccagccctgggcctcgacctgttgctcaactgctcactgttgctgtgccggggtggtggccacccactggacctgctctccgtgacgctggcctcgggctcccgctgtttctccttcctgtctgtggcctggggcttcgtgtcagatgtggatatccagagcgagcgcttcagggccttgggcagtgcccgcttcacactgggcacggtgctgggcctcgccacactgcacacctaccgcggacgcctctcctacctccccgccactgtggaacctgcctcgcccacccctgcccatagcctgcctcgtgccaagtcggagctgaccctaaccccagacccagccccgcccatggcccactcacccctgcatcgttctgtgtctgacctgcctcttcccctgccccagcctgccctggcctctcctggctcgccagaacccctgcccatcctgtccctcaacggtgggggcccagagctggctggggactggggtggggctggggatgctccgctgtccccggacccactgctgtcttcacctcctggctctcccaaggcagctctacactcacccgtctccgaaggggcccccgtaattcccccatcctctgggctcccacttcccacccctgatgcccgggtaggggcctccacctgcggcccgcccgaccacctgctgcctccgctgggcaccccgctgcccccagactgggtgacgctggagggggactttgtgctcatgttggccatctcgcccagccacctaggcgctgacctggtggcagctccgcatgcgcgcttcgacgacggcctggtgcacctgtgctgggtgcgtagcggcatctcgcgggctgcgctgctgcgccttttcttggccatggagcgtggtagccacttcagcctgggctgtccgcagctgggctacgccgcggcccgtgccttccgcctagagccgctcacaccacgcggcgtgctcacagtggacggggagcaggtggagtatgggccgctacaggcacagatgcaccctggcatcggtacactgctcactgggcctcctggctgcccggggcgggagccctga

SEQ ID NO: 26 - DNA, 1614 bp, Homo sapiens
  source 1..1614: mol_type = other DNA
atgggggcgacgggggcggcggagccgctgcaatccgtgctgtgggtgaagcagcagcgctgcgccgtgagcctggagcccgcgcgggctctgctgcgctggtggcggagcccggggcccggagccggcgcccccggcgcggatgcctgctctgtgcctgtatctgagatcatcgccgttgaggaaacagacgttcacgggaaacatcaaggcagtggaaaatggcagaaaatggaaaagccttacgcttttacagttcactgtgtaaagagagcacgacggcaccgctggaagtgggcgcaggtgactttctggtgtccagaggagcagctgtgtcacttgtggctgcagaccctgcgggagatgctggagaagctgacgtccagaccaaagcatttactggtatttatcaacccgtttggaggaaaaggacaaggcaagcggatatatgaaagaaaagtggcaccactgttcaccttagcctccatcaccactgacatcatcgttactgaacatgctaatcaggccaaggagactctgtatgagattaacatagacaaatacgacggcatcgtctgtgtcggcggagatggtatgttcagcgaggtgctgcacggtctgattgggaggacgcagaggagcgccggggtcgaccagaaccacccccgggctgtgctggtccccagtagcctccggattggaatcattcccgcagggtcaacggactgcgtgtgttactccaccgtgggcaccagcgacgcagaaacctcggcgctgcatatcgttgttggggactcgctggccatggatgtgtcctcagtccaccacaacagcacactccttcgctactccgtgtccctgctgggctacggcttctacggggacatcatcaaggacagtgagaagaaacggtggttgggtcttgccagatacgacttttcaggtttaaagaccttcctctcccaccactgctatgaagggacagtgtccttcctccctgcacaacacacggtgggatctccaagggataggaagccctgccgggcaggatgctttgtttgcaggcaaagcaagcagcagctggaggaggagcagaagaaagcactgtatggtttggaagctgcggaggacgtggaggagtggcaagtcgtctgtgggaagtttctggccatcaatgccacaaacatgtcctgtgcttgtcgccggagccccaggggcctctccccggctgcccacttgggagacgggtcttctgacctcatcctcatccggaaatgctccaggttcaattttctgagatttctcatcaggcacaccaaccagcaggaccagtttgacttcacttttgttgaagtttatcgcgtcaagaaattccagtttacgtcgaagcacatggaggatgaggacagcgacctcaaggagggggggaagaagcgctttgggcacatttgcagcagccacccctcctgctgctgcaccgtctccaacagctcctggaactgcgacggggaggtcctgcacagccctgccatcgaggtcagagtccactgccagctggttcgactctttgcacgaggaattgaagagaatccgaagccagactcacacagctga

SEQ ID NO: 27 - protein, 395 aa, Homo sapiens
MPGRSCVALVLLAAAVSCAVAQHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 28 - protein, 411 aa, Homo sapiens
MNCCIGLGEKARGSHRASYPSLSALFTEASILGFGSFAVKAQWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 29 - protein, 389 aa, Homo sapiens
MNCCIGLGEKARGSHRASYPSLSALFTEASILGFGSFAVKAQWTEDCRKSTYPPSGPTVFPAVIRYRGAVPWYTINLDLPPYKRWHELMLDKAPVPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW